(19) United States
(12) Patent Application Publication
(10) Pub. No.: US 2026/0253433 A1
(43) Pub. Date: Aug. 27, 2026

(54) SPATIAL OMICS SINGLE CELL DATA ACQUISITION METHOD AND APPARATUS, AND ELECTRONIC DEVICE

(71) Applicants: BGI SHENZHEN, Shenzhen, Guangdong (CN); BGI SHENZHEN CO., LTD, Shenzhen, Guangdong (CN)

(72) Inventors: Mei LI, Shenzhen, Guangdong (CN); Huanlin LIU, Shenzhen, Guangdong (CN); Min LI, Shenzhen, Guangdong (CN); Weixuan CEN, Shenzhen, Guangdong (CN); Zirui HUANG, Shenzhen, Guangdong (CN); Zhonghan DENG, Shenzhen, Guangdong (CN); Kui SU, Shenzhen, Guangdong (CN); Zepeng LI, Shenzhen, Guangdong (CN); Ning FENG, Shenzhen, Guangdong (CN); Qichen JIANG, Shenzhen, Guangdong (CN); Yiwen WU, Shenzhen, Guangdong (CN); Qiang KANG, Shenzhen, Guangdong (CN); Yuxiang LI, Shenzhen, Guangdong (CN); Yong ZHANG, Shenzhen, Guangdong (CN); Xun XU, Shenzhen, Guangdong (CN)

(21) Appl. No.: 18/879,230

(22) PCT Filed: Dec. 5, 2022
(86) PCT No.: PCT/CN2022/136698

§ 371 Date: Dec. 26, 2024

(30) Foreign Application Priority Data
Jun. 29, 2022 (WO) PCT/CN2022/102360
Jun. 29, 2022 (WO) PCT/CN2022/102424
Jun. 29, 2022 (WO) PCT/CN2022/102443
Jun. 29, 2022 (WO) PCT/CN2022/102505
Aug. 8, 2022 (WO) PCT/CN2022/110779

(51) Int. Cl. G06V 20/69; G01N 15/01; G01N 15/1434; G06T 3/14; G06T 3/20; G06T 3/40; G06T 7/187; G06T 7/33; G06T 7/73; G06V 10/10; G06V 10/26; G06V 10/28; G06V 10/764; G06V 10/82

(52) U.S. Cl. CPC G06V 20/698; G01N 15/01; G01N 15/1434; G06T 3/14; G06T 3/20; G06T 3/40; G06T 7/187; G06T 7/33; G06T 7/73; G06V 10/16; G06V 10/26; G06V 10/28; G06V 10/764; G06V 10/82; G06V 20/693; G06V 20/695; G06T 2207/10056; G06T 2207/20084; G06T 2207/30024

(57) ABSTRACT

A spatial omics single cell data acquisition method includes: acquiring a dyeing image of a biological sample, and acquiring a visual gene expression image of the biological sample; registering the dyeing image with the visual gene expression image, so as to obtain a registered image; performing cell segmentation on the registered image, so as to obtain a cell mask image; and according to the cell mask image, determining a cell of a background gene molecule of an unknown cell in the visual gene expression image, and marking the cell, so as to obtain a spatial omics molecular expression matrix with a cell mark. Compared with the prior art, the present disclosure relates to obtaining high-precision spatial single cell data by means of a gene expression matrix diagram, and a processing flow involving splicing, registration, segmentation and correction.

### CROSS-REFERENCE TO RELATED APPLICATIONS

This application is based on PCT Patent Application No. PCT/CN2022/102505, filed on Jun. 29, 2022, PCT Patent Application No. PCT/CN2022/102424, filed on Jun. 29, 2022, PCT Patent Application No. PCT/CN2022/102360, filed on Jun. 29, 2022, PCT Patent Application No. PCT/CN2022/102443, filed on Jun. 29, 2022, and PCT Patent Application No. PCT/CN2022/110779, filed on Aug. 8, 2022, and claims priority to and the benefit of the above identified PCT Patent applications, the entire content of which is incorporated herein by reference.

### FIELD

The present disclosure relates to the technical field of data processing, in particular to a method for obtaining spatial omics single cell data, and a device, and an electronic device thereof.

### BACKGROUND

Spatial transcriptomics technology has the potential to uncover the functions of the internal spaces within tissue structures at different stages and reveal the heterogeneity among different tissue structures. This advancement paves the way for a deeper understanding of tissue structures and intercellular communication of organisms. While several spatial transcriptomics technologies have been developed to enable an entire transcriptome capture, the resolution of the expression information matrix they provide are usually not sufficient for single-cell analysis. Consequently, there is an urgent need to realize the acquisition of single-cell data.

### SUMMARY

Embodiments of the present disclosure provide a method for obtaining spatial omics single cell data, and a device, an electronic device, and a storage medium thereof.

According to the embodiments of the present disclosure in a first aspect, the method for obtaining spatial omics single cell data includes:
obtaining a stained image of a biological sample and a visualization image of gene expression of the biological sample;
performing image registration for the stained image and the visualization image of gene expression to obtain a registered image;
performing cell segmentation for the registered image to obtain a cell mask image; and
determining cell-belonging for a background gene molecule unknown of the cell-belonging in the visualization image of gene expression based on the cell mask image, and labeling a cell with the determined cell-belonging for the background gene molecule, to obtain an expression matrix of spatial omics molecules with the cell label.

In some embodiments, said obtaining a stained image of a biological sample includes: obtaining the stained image of the biological sample with a microscopy; or
obtaining a stitched stained image of the biological sample with a microscope, wherein the stitched stained image is stitched from a plurality of sub-stained images; or
obtaining a plurality of sub-stained images of the biological sample with a microscopy, and stitching the plurality of sub-stained images to obtain the stitched stained image.

In some embodiments, said obtaining a stained image of a biological sample includes:
obtaining the plurality of sub-stained images of the biological sample with a microscopy;
performing first stitching for the plurality of sub-stained images of the biological sample to obtain a first stained image;
determining whether to perform second stitching for the plurality of sub-stained images of the biological sample based on an image quality of the first stained image;
identifying the first stained image as the stained image in response to a negative result of the determination; and
performing the second stitching for the plurality of sub-stained images of the biological sample to obtain the stained image in response to a positive result of the determination.

In some embodiments, said obtaining a plurality of sub-stained images of the biological
sample with a microscopy, and stitching the plurality of sub-stained images to obtain the stitched stained image include:
determining an overlapping region between each of the plurality of sub-stained images and a template image;
obtaining a plurality of overlapping sub-image pairs by matching a plurality of
overlapping sub-stained images and a plurality of overlapping sub-template images, wherein a section corresponding to the determined overlapping region in each of the plurality of sub-stained images and a section corresponding to the determined overlapping region in the template image are cropped respectively, to provide the plurality of overlapping sub-stained images and the plurality of overlapping sub-template images that are identical by numbers;
obtaining an offset between each of the plurality of sub-stained images and the corresponding sub-template image by performing frequency domain calculations on the plurality of overlapping sub-image pairs; and
translating each of the plurality of sub-stained images by the offset toward the corresponding sub-template image, and stitching the plurality of sub-stained images together with the template image.

In some embodiments, said obtaining a plurality of sub-stained images of the biological sample with a microscopy, and stitching the plurality of sub-stained images to obtain the stitched stained image include:
obtaining the plurality of sub-stained images and template information about a track line or a track cross for the biological sample;
performing a pre-stitching for the plurality of sub-stained images to obtain respective pre-stitched coordinates of the plurality of sub-stained images;
selecting at least one first reference image from the plurality of sub-stained images according to a feature of the track line or the track cross;
deriving a global template according to the template information of the biological sample and the pre-stitched coordinates of the first reference image;
for a second reference image in the plurality of sub-stained images, calculating an offset between pre-stitched coordinates of the second reference image and corresponding template coordinates of the second reference image in the global template, wherein second reference image is different from the first reference image; and
adjusting the coordinates of the second reference image by the offset and stitching the second reference image for generation of the stitched stained image.

In some embodiments, said performing image registration for the stained image and the visualization image of gene expression to obtain a registered image includes:
obtaining a track line in the stained image and rotating the stained image in an angle between the track line and a horizontal direction, wherein the stained image and the visualization image of gene expression each include a plurality of track lines and/or a plurality of track crosses, two of the track lines intersect to form the track crosses, and each of the plurality of track lines is corresponded with an index number;
obtaining a third reference image by scaling the stained image, wherein the third reference image has the same scale as the visualization image of gene expression;
determining an object offset based on the visualization image of gene expression and the third reference image; and
translating the third reference image by the object offset to obtain the registered image.

In some embodiments, after said obtaining a third reference image by scaling the stained image, the method further includes:
determining a similarity between the third reference image and the visualization image of gene expression; and
taking a position with a maximum similarity between the third reference image and the visualization image of gene expression as a result of a coarse registration.

In some embodiments, said performing image registration for the stained image and the visualization image of gene expression to obtain a registered image includes:
obtaining the stained image and the visualization image of gene expression each including a plurality of track lines and/or a plurality of track crosses, two of the track lines intersect to form the track crosses, and each of the plurality of track lines is corresponded with an index number;
performing a preprocessing for the stained image to obtain a candidate image set;
determining a binary image set corresponding to the candidate image set;
determining a track cross pair(s) having a correspondence between the binary image set and the visualization image of gene expression, based on a similarity between the binary image set and the visualization image of gene expression;
determining an offset information based on the track cross pair; and
processing an object image based on the offset information to obtain the registered image, wherein the object image is an image from the candidate image set and corresponding to the track cross pair.

In some embodiments, before said performing a cell segmentation for the registered image to obtain a cell mask image, the method further includes:
performing a tissue segmentation for the registered image to obtain a tissue mask image.

In some embodiments, said performing a tissue segmentation for the registered image to obtain a tissue mask image includes:
obtaining a gene expression of an object tissue to be segmented in the registered image to determine a gene expression matrix of the registered image, wherein the gene expression in the gene expression matrix is arranged according to a pixel position;
determining image data by assigning the gene expression at each pixel position in the gene expression matrix to a corresponding pixel position in the registered image;
performing an image processing for the image data to determine a foreground pixel point of the image data; and
labeling connected components for the image data based on the foreground pixel point to obtain the tissue mask image.

In some embodiments, after said performing a tissue segmentation for the registered image to obtain a tissue mask image, the method further includes:
obtaining an outer boundary image and an inner boundary image in a grayscale image corresponding to the tissue mask image by calculating respectively;
dividing the outer boundary image and the inner boundary image into N of segments respectively, and matching each segment of the outer boundary image with each segment of the inner boundary image by pair to obtain N image combinations, wherein each of the image combinations includes one segment of the outer boundary image and one segment of the inner boundary image;
calculating a ratio of pixel density between one segment of the outer boundary image and one segment of the inner boundary image in each of the image combinations, respectively, to obtain N ratios of pixel density, wherein the ratio of pixel density is a ratio of pixel occupancy of one segment of the outer boundary image to one segment of the inner boundary image in each of the image combinations, and the pixel occupancy is a ratio of pixel points to total pixel points; and
calculating a quality of the grayscale image corresponding to the tissue mask image based on the ratio of pixel density.

In some embodiments, said performing a cell segmentation for the registered image to obtain a cell mask image includes:
inputting the registered image into an image segmentation model to obtain a segmentation result of individual pixel points in the registered image, wherein the segmentation result includes a pixel point belonging to a cell; and
outputting a segmentation result of the registered image to obtain the cell mask image.

In some embodiments, said performing a cell segmentation for the registered image to obtain a cell mask image includes:
preprocessing the visualization image of gene expression to obtain a preprocessed image;
performing a binaryzation processing for the preprocessed image to obtain an initial mask image; and
based on the initial mask image, segmenting a connected component with a presence of cell adhesion using a distance transform-based watershed algorithm to obtain a segmented cell mask image.

In some embodiments, said performing a cell segmentation for the registered image to obtain a cell mask image includes:
determining a labeled region and a cell area corresponding to each tissue type of said target object based on the stained image;
determining an area of a sub-region to be processed in each labeled region based on the cell area, wherein the area of the sub-region to be processed is in a positive correlation with the cell area, and the sub-region to be processed characterizes the minimum processing unit of the corresponding labeled region;
performing a grid processing for the corresponding labeled region based on the area of each said sub-region to be processed to obtain gridded coordinates corresponding to each said labeled region; and
extracting data corresponding to the gridded coordinates from the visualization image of gene expression and superimposing the data to the gridded coordinates to obtain a cell processing result of the target object.

In some embodiments, said determining a cell-belonging for a background gene molecule unknown of the cell-belonging in the visualization image of gene expression based on the cell mask image includes:
classifying, based on the cell mask image, individual gene expression features of the visualization image of gene expression as a cellular gene expression feature located inside a cell and a background gene expression feature located outside the cell;
fitting a probability distribution model with individual gene expression features using a spatial location and a signal value of the gene expression feature;
obtaining, based on the probability distribution model, a probability value showing that a target background gene expression feature, which is located within a predetermined range of a target cell belongs to the target cell; and
outputting a correction result based on the probability value for correction of the background gene expression feature.

In some embodiments, said determining a cell-belonging for a background gene molecule unknown of the cell-belonging in the visualization image of gene expression based on the cell mask image includes:
determining an initial gene molecule belonging to a target cell and the background gene molecule unknown of the cell-belonging based on the visualization image of gene expression;
acquiring a coordinate information of the initial gene molecule;
determining center point coordinates of said target cell based on the coordinate information;
constructing a Voronoi diagram based on the center point coordinates and the coordinate information; and
determining the cell-belonging for the background gene molecule unknown of the cell-belonging within the Voronoi diagram.

In some embodiments, said determining a cell-belonging for a background gene molecule unknown of the cell-belonging in the visualization image of gene expression based on the cell mask image includes:
determining an initial molecule number of gene molecules belonging to a target cell and an initial boundary area of a target region occupied by the gene molecules belonging to the target cell in the visualization image of gene expression;
determining a density of the gene molecules belonging to the target cell based on the molecule number and the initial boundary area;
determining a molecule number of background gene molecules within a predetermined range of the target region and an area of the predetermined range;
determining a density of the background gene molecules based on the molecule number of the background gene molecules and the area of the predetermined range of the background gene molecule; and
determining the cell-belonging for the background gene molecule based on the density of the gene molecules versus the density of the background gene molecules.

In some embodiments, said determining a cell-belonging for a background gene molecule unknown of the cell-belonging in the visualization image of gene expression based on the cell mask image includes:
determining a target region occupied by a gene molecule belonging to a target cell in the visualization image of gene expression;
determining spatial coordinates of the background gene molecule unknown of the cell-belonging within a predetermined correction range of the target region and a signal value of a gene expression feature thereof;
inputting the spatial coordinates and the signal value of the gene expression feature into a probability distribution model, to obtain a probability value showing that the background gene molecule belongs to the target cell, wherein the probability distribution model is fitted with the spatial coordinates and the signal value of the gene expression feature for the gene molecule belonging to the target cell; and
determining the cell-belonging for the background gene molecule based on the probability score.

In some embodiments, said labeling a cell with the determined cell-belonging includes:
staining the cell with the determined cell-belonging by a ssDNA staining solution and a cell boundary fluorescent staining solution, wherein the ssDNA staining solution includes a buffer and a ssDNA reagent, and the cell boundary fluorescent staining solution includes a fluorescent dye.

In some embodiments, the method further includes:
obtaining a stained image to be tested or a visualization image of gene expression to be tested; and
inputting the stained image to be tested or the visualization image of gene expression to be tested into a directed object detection network to obtain an object detection result,
wherein the directed object detection network includes:
a backbone layer configured to perform a feature extraction on the stained image and the visualization image of gene expression, to obtain a plurality of feature images at different scales;
a neck layer configured to receive the plurality of feature images from the backbone layer and perform a cross-scale fusion for the plurality of feature images, to obtain a plurality of fused feature images; and
a head layer configured to perform a prediction for the plurality of fused feature images, to obtain the object detection result with respect to the stained image to be tested or the visualization image of gene expression to be tested,
wherein the object detection result includes: a prediction information of an object box coordinate, an object prediction information and an angle prediction information.

In some embodiments, wherein a plane where the stained image is located includes a first direction and a second direction perpendicular to the first direction, the stained image includes a plurality of first straight track lines transverse to the second direction and a plurality of second straight track lines transverse to the first direction,
wherein coordinates of points in the stained image are determined by:
a step of sampling, including:
performing first-direction equal-interval sampling for the stained image in a first direction by applying a first sampling strip to determine a first candidate point set, wherein each time of said first-direction equal-interval sampling includes:
calculating a cumulative sum of pixel values in a first direction for pixels within a sampled region of the stained image selected during this time of said first-direction equal-interval sampling by the first sampling strip, to obtain a series of first pixel value sums; and determining a first extreme point of the series of first pixel value sums, and determining,
for each first extreme point determined, a first candidate point obtained from this time of said first-direction equal-interval sampling, wherein a coordinate of the first candidate point in the first direction is determined according to a location of the first sampling strip in the first direction during this time of said first-direction equal-interval sampling, a coordinate of the first candidate point in the second direction is determined according to a coordinate in the second direction corresponding to a location of the first extreme point in the second direction,
wherein the first candidate point set consists of the first candidate points obtained from individual times of said first-direction equal interval sampling;
performing second-direction equal interval sampling for the stained image in a second direction by applying a second sampling strip to determine a second candidate point set, wherein each time of said second-direction equal interval sampling includes:
calculating a cumulative sum of pixel values in a second direction for pixels within a sampled region of the stained image selected during this time of said second-direction equal-interval sampling by the second sampling strip, to obtain a series of second pixel value sums; and
determining a second extreme point of the series of second pixel value sums, and determining, for each second extreme point determined, a second candidate point obtained from this time of said second-direction equal interval sampling, wherein a coordinate of the second candidate point in the second direction is determined according to a location of the second sampling strip in the second direction during this time of said second-direction equal-interval sampling, a coordinate of the second candidate point in the first direction is determined according to a coordinate in the first direction corresponding to a location of the second extreme point in the first direction,
wherein the second candidate point set consists of the second candidate points obtained from individual times of said second-direction equal interval sampling;
a step of grouping, including:
dividing the first candidate points in the first candidate point set into a plurality of first candidate point groups; and
dividing the second candidate points in the second candidate point set into a plurality of second candidate point groups;
a step of linear fitting, including:
performing a first linear fit for the first candidate points in individual first candidate point groups to obtain respective first linear analytic formulas; and
performing a second linear fit for the second candidate points in individual second candidate point groups to obtain respective second linear analytic formulas; and
a step of coordinate calculation, including:
calculating coordinates of respective intersections of individual first linear formulas with individual second linear formula.

In some embodiments, after labeling cell with the determined cell-belonging for the background gene molecule to obtain an expression matrix of spatial omics molecules with the cell label, the method includes:
obtaining a first image of the biological sample, wherein the first image includes a stained cell boundary of the biological sample;
obtaining a second image of the biological sample, wherein the second image includes a stained genetic material of the biological sample;
obtaining information about a gene expression level of the biological sample;
registering the second image with the first image to obtain a registered image of the second image with the first image;
registering the second image with the information about the gene expression level to obtain a registered image of the second image with the information about the gene expression level;
obtaining a registered image of the first image with the information about the gene expression level based on the registered image of the second image with the first image and the registered image of the second image with the information about the gene expression level;
obtaining a cell boundary of the biological sample based on the registered image of the first image with the information about the gene expression level;

obtaining gene expression information of a cell of the biological sample based on the cell boundary and the registered image of the first image with the information about the gene expression level.

According to the embodiments of the present disclosure in a second aspect, the device for obtaining spatial omics single cell data includes:
an obtaining unit for obtaining a stained image of a biological sample and a visualization image of gene expression of the biological sample;
a registration unit for performing image registration for the stained image and the visualization image of gene expression to obtain a registered image;
a first segmentation unit for performing cell segmentation for the registered image to obtain a cell mask image;
a correction unit for determining cell-belonging for a background gene molecule unknown of the cell-belonging in the visualization image of gene expression based on the cell mask image; and
a labeling unit for labeling a cell with the determined cell-belonging for the background gene molecule, to obtain an expression matrix of spatial omics molecules with the cell label.

In some embodiments, said obtaining unit is further used for:
obtaining the stained image of the biological sample with a microscopy; or
obtaining a stitched stained image of the biological sample with a microscope, wherein the stitched stained image is stitched from a plurality of sub-stained images; or
obtaining a plurality of sub-stained images of the biological sample with a microscopy, and stitching the plurality of sub-stained images to obtain the stitched stained image.

In some embodiments, said obtaining unit includes:
a first obtaining module for obtaining the plurality of sub-stained images of the biological sample with a microscopy and performing first stitching for the plurality of sub-stained images of the biological sample to obtain a first stained image;
a determining module for determining whether to perform second stitching for the plurality of sub-stained images of the biological sample based on an image quality of the first stained image;
a identifying module for identifying the first stained image as the stained image in response to a negative result of the determination; and
a second obtaining module for performing the second stitching for the plurality of sub-stained images of the biological sample to obtain the stained image in response to a positive result of the determination.

In some embodiments, said first obtaining module is further used for:
determining an overlapping region between each of the plurality of sub-stained images and a template image;
obtaining a plurality of overlapping sub-image pairs by matching a plurality of overlapping sub-stained images and a plurality of overlapping sub-template images, wherein a section corresponding to the determined overlapping region in each of the plurality of sub-stained images and a section corresponding to the determined overlapping region in the template image are cropped respectively, to provide the plurality of overlapping sub-stained images and the plurality of overlapping sub-template images that are identical by numbers;
obtaining an offset between each of the plurality of sub-stained images and the corresponding sub-template image by performing frequency domain calculations on the plurality of overlapping sub-image pairs; and
translating each of the plurality of sub-stained images by the offset toward the corresponding sub-template image, and stitching the plurality of sub-stained images together with the template image.

In some embodiments, said first obtaining module is further used for:
obtaining the plurality of sub-stained images and template information about a track line or a track cross for the biological sample;
performing a pre-stitching for the plurality of sub-stained images to obtain respective pre-stitched coordinates of the plurality of sub-stained images;
selecting at least one first reference image from the plurality of sub-stained images according to a feature of the track line or the track cross;
deriving a global template according to the template information of the biological sample and the pre-stitched coordinates of the first reference image;
for a second reference image in the plurality of sub-stained images, calculating an offset between pre-stitched coordinates of the second reference image and corresponding template coordinates of the second reference image in the global template, wherein second reference image is different from the first reference image; and
adjusting the coordinates of the second reference image by the offset and stitching the second reference image for generation of the stitched stained image.

In some embodiments, said registration unit includes:
an obtaining module for obtaining a track line in the stained image and rotating the stained image in an angle between the track line and a horizontal direction, wherein the stained image and the visualization image of gene expression each include a plurality of track lines and/or a plurality of track crosses, two of the track lines intersect to form the track crosses, and each of the plurality of track lines is corresponded with an index number;
a processing module for obtaining a third reference image by scaling the stained image, wherein the third reference image has the same scale as the visualization image of gene expression;
a first determining module for determining an object offset based on the visualization image of gene expression and the third reference image; and
a registration module for translating the third reference image by the object offset to obtain the registered image.

In some embodiments, said registration unit further includes:
a second determining module for determining a similarity between the third reference image and the visualization image of gene expression; and
a coarse registration taking a position with a maximum similarity between the third reference image and the visualization image of gene expression as a result of a coarse registration.

In some embodiments, said registration unit is further used for:
obtaining the stained image and the visualization image of gene expression each including a plurality of track lines and/or a plurality of track crosses, two of the track lines intersect to form the track crosses, and each of the plurality of track lines is corresponded with an index number; performing a preprocessing for the stained image to obtain a candidate image set;
determining a binary image set corresponding to the candidate image set;
determining a track cross pair(s) having a correspondence between the binary image set and the visualization image of gene expression, based on a similarity between the binary image set and the visualization image of gene expression;
determining an offset information based on the track cross pair; and
processing an object image based on the offset information to obtain the registered image, wherein the object image is an image from the candidate image set and corresponding to the track cross pair.

In some embodiments, said device further includes:
a second segmentation unit for performing a tissue segmentation for the registered image to obtain a tissue mask image, before said performing a cell segmentation for the registered image to obtain a cell mask image.

In some embodiments, said second segmentation unit is used for:
obtaining a gene expression of an object tissue to be segmented in the registered image to determine a gene expression matrix of the registered image, wherein the gene expression in the gene expression matrix is arranged according to a pixel position;
determining image data by assigning the gene expression at each pixel position in the gene expression matrix to a corresponding pixel position in the registered image;
performing an image processing for the image data to determine a foreground pixel point of the image data; and
labeling connected components for the image data based on the foreground pixel point to obtain the tissue mask image.

In some embodiments, the device further includes:
a calculating unit for obtaining an outer boundary image and an inner boundary image in a grayscale image corresponding to the tissue mask image by calculating respectively;
a matching unit for dividing the outer boundary image and the inner boundary image into N of segments respectively, and matching each segment of the outer boundary image with each segment of the inner boundary image by pair to obtain N image combinations, wherein each of the image combinations includes one segment of the outer boundary image and one segment of the inner boundary image;
the calculating unit is further used for calculating a ratio of pixel density between one segment of the outer boundary image and one segment of the inner boundary image in each of the image combinations, respectively, to obtain N ratios of pixel density, wherein the ratio of pixel density is a ratio of pixel occupancy of one segment of the outer boundary image to one segment of the inner boundary image in each of the image combinations, and the pixel occupancy is a ratio of pixel points to total pixel points; and
the calculating unit is further used for calculating a quality of the grayscale image corresponding to the tissue mask image based on the ratio of pixel density.

In some embodiments, the first segmentation unit is further used for:
inputting the registered image into an image segmentation model to obtain a segmentation result of individual pixel points in the registered image, wherein the segmentation result includes a pixel point belonging to a cell; and
outputting a segmentation result of the registered image to obtain the cell mask image.

In some embodiments, the first segmentation unit is further used for:
preprocessing the visualization image of gene expression to obtain a preprocessed image;
performing a binaryzation processing for the preprocessed image to obtain an initial mask image; and
based on the initial mask image, segmenting a connected component with a presence of cell adhesion using a distance transform-based watershed algorithm to obtain a segmented cell mask image.

In some embodiments, the first segmentation unit is further used for:
determining a labeled region and a cell area corresponding to each tissue type of said target object based on the stained image;
determining an area of a sub-region to be processed in each labeled region based on the cell area, wherein the area of the sub-region to be processed is in a positive correlation with the cell area, and the sub-region to be processed characterizes the minimum processing unit of the corresponding labeled region;
performing a grid processing for the corresponding labeled region based on the area of each said sub-region to be processed to obtain gridded coordinates corresponding to each said labeled region; and
extracting data corresponding to the gridded coordinates from the visualization image of gene expression and superimposing the data to the gridded coordinates to obtain a cell processing result of the target object.

In some embodiments, the correction unit is further used for:
classifying, based on the cell mask image, individual gene expression features of the visualization image of gene expression as a cellular gene expression feature located inside a cell and a background gene expression feature located outside the cell;
fitting a probability distribution model with individual gene expression features using a spatial location and a signal value of the gene expression feature;
obtaining, based on the probability distribution model, a probability value showing that a target background gene expression feature, which is located within a predetermined range of a target cell belongs to the target cell; and
outputting a correction result based on the probability value for correction of the background gene expression feature.

In some embodiments, the correction unit is further used for:
determining an initial gene molecule belonging to a target cell and the background gene molecule unknown of the cell-belonging based on the visualization image of gene expression;
acquiring a coordinate information of the initial gene molecule;
determining center point coordinates of said target cell based on the coordinate information;
constructing a Voronoi diagram based on the center point coordinates and the coordinate information; and
determining the cell-belonging for the background gene molecule unknown of the cell-belonging within the Voronoi diagram.

In some embodiments, the correction unit is further used for:
determining an initial molecule number of gene molecules belonging to a target cell and an initial boundary area of a target region occupied by the gene molecules belonging to the target cell in the visualization image of gene expression;
determining a density of the gene molecules belonging to the target cell based on the molecule number and the initial boundary area;
determining a molecule number of background gene molecules within a predetermined range of the target region and an area of the predetermined range;
determining a density of the background gene molecules based on the molecule number of the background gene molecules and the area of the predetermined range of the background gene molecule; and
determining the cell-belonging for the background gene molecule based on the density of the gene molecules versus the density of the background gene molecules.

In some embodiments, the correction unit is further used for:
determining a target region occupied by a gene molecule belonging to a target cell in the visualization image of gene expression;
determining spatial coordinates of the background gene molecule unknown of the cell-belonging within a predetermined correction range of the target region and a signal value of a gene expression feature thereof;
inputting the spatial coordinates and the signal value of the gene expression feature into a probability distribution model, to obtain a probability value showing that the background gene molecule belongs to the target cell, wherein the probability distribution model is fitted with the spatial coordinates and the signal value of the gene expression feature for the gene molecule belonging to the target cell; and
determining the cell-belonging for the background gene molecule based on the probability score.

In some embodiments, the labeling unit is used for:
staining the cell with the determined cell-belonging by a ssDNA staining solution and a cell boundary fluorescent staining solution, wherein the ssDNA staining solution includes a buffer and a ssDNA reagent, and the cell boundary fluorescent staining solution includes a fluorescent dye.

In some embodiments, the device further includes a detection unit for:
obtaining a stained image to be tested or a visualization image of gene expression to be tested; and
inputting the stained image to be tested or the visualization image of gene expression to be tested into a directed object detection network to obtain an object detection result,
wherein the directed object detection network includes:
a backbone layer configured to perform a feature extraction on the stained image and the visualization image of gene expression, to obtain a plurality of feature images at different scales;
a neck layer configured to receive the plurality of feature images from the backbone layer and perform a cross-scale fusion for the plurality of feature images, to obtain a plurality of fused feature images; and
a head layer configured to perform a prediction for the plurality of fused feature images, to obtain the object detection result with respect to the stained image to be tested or the visualization image of gene expression to be tested,
wherein the object detection result includes: a prediction information of an object box coordinate, an object prediction information and an angle prediction information.

In some embodiments, wherein a plane where the stained image is located includes a first direction and a second direction perpendicular to the first direction, the stained image includes a plurality of first straight track lines transverse to the second direction and a plurality of second straight track lines transverse to the first direction,
wherein coordinates of points in the stained image are determined by:
a step of sampling, including:
performing first-direction equal-interval sampling for the stained image in a first direction by applying a first sampling strip to determine a first candidate point set, wherein each time of said first-direction equal-interval sampling includes:
calculating a cumulative sum of pixel values in a first direction for pixels within a sampled region of the stained image selected during this time of said first-direction equal-interval sampling by the first sampling strip, to obtain a series of first pixel value sums; and
determining a first extreme point of the series of first pixel value sums, and determining, for each first extreme point determined, a first candidate point obtained from this time of said first-direction equal-interval sampling, wherein a coordinate of the first candidate point in the first direction is determined according to a location of the first sampling strip in the first direction during this time of said first-direction equal-interval sampling, a coordinate of the first candidate point in the second direction is determined according to a coordinate in the second direction corresponding to a location of the first extreme point in the second direction,
wherein the first candidate point set consists of the first candidate points obtained from individual times of said first-direction equal interval sampling;
performing second-direction equal interval sampling for the stained image in a second direction by applying a second sampling strip to determine a second candidate point set, wherein each time of said second-direction equal interval sampling includes:
calculating a cumulative sum of pixel values in a second direction for pixels within a sampled region of the stained image selected during this time of said second-direction equal-interval sampling by the second sampling strip, to obtain a series of second pixel value sums; and
determining a second extreme point of the series of second pixel value sums, and determining, for each second extreme point determined, a second candidate point obtained from this time of said second-direction equal interval sampling, wherein a coordinate of the second candidate point in the second direction is determined according to a location of the second sampling strip in the second direction during this time of said second-direction equal-interval sampling, a coordinate of the second candidate point in the first direction is determined according to a coordinate in the first direction corresponding to a location of the second extreme point in the first direction,
wherein the second candidate point set consists of the second candidate points obtained from individual times of said second-direction equal interval sampling;
a step of grouping, including:
dividing the first candidate points in the first candidate point set into a plurality of first candidate point groups; and
dividing the second candidate points in the second candidate point set into a plurality of second candidate point groups;
a step of linear fitting, including:
performing a first linear fit for the first candidate points in individual first candidate point groups to obtain respective first linear analytic formulas; and
performing a second linear fit for the second candidate points in individual second candidate point groups to obtain respective second linear analytic formulas; and
a step of coordinate calculation, including:
calculating coordinates of respective intersections of individual first linear formulas with individual second linear formula.

In some embodiments, the device further includes an analyzing unit for:
obtaining a first image of the biological sample, wherein the first image includes a stained cell boundary of the biological sample;
obtaining a second image of the biological sample, wherein the second image includes a stained genetic material of the biological sample;
obtaining information about a gene expression level of the biological sample;
registering the second image with the first image to obtain a registered image of the second image with the first image;
registering the second image with the information about the gene expression level to obtain a registered image of the second image with the information about the gene expression level;
obtaining a registered image of the first image with the information about the gene expression level based on the registered image of the second image with the first image and the registered image of the second image with the information about the gene expression level;
obtaining a cell boundary of the biological sample based on the registered image of the first image with the information about the gene expression level;
obtaining gene expression information of a cell of the biological sample based on the cell boundary and the registered image of the first image with the information about the gene expression level.

According to the embodiments of the present disclosure in a third aspect, the electronic device includes:
a processor, and
a memory communicatively connected to said at least one processor, wherein
the memory having stored therein instructions that, when executed by a processor of an electronic device, causes the electronic device to perform a method according to any embodiment of the present disclosure in the first aspect.

According to the embodiments of the present disclosure in a forth aspect, the non-transitory computer-readable storage medium having stored therein instructions that, when executed by a processor of an electronic device, causes the electronic device to perform a method according to any embodiment of the present disclosure in the first aspect.

According to the embodiments of the present disclosure in a fifth aspect, the computer program product including a computer program, wherein when executed by a processor, the computer program product implements a method according to any embodiment of the present disclosure in the first aspect.

According to the embodiments of the present disclosure, the method for obtaining spatial omics single cell data, and a device, and an electronic device, and a storage medium thereof obtain a stained image of a biological sample and a visualization image of gene expression of the biological sample; perform image registration for the stained image and the visualization image of gene expression to obtain a registered image; perform cell segmentation for the registered image to obtain a cell mask image; and determine cell-belonging for a background gene molecule unknown of the cell-belonging in the visualization image of gene expression based on the cell mask image, and labeling a cell with the determined cell-belonging for the background gene molecule, to obtain an expression matrix of spatial omics molecules with the cell label. Compared with the related art, the embodiments of the present disclosure realize the obtaining of highly accurate spatial single-cell data by the processing step of gene expression matrix image processing, cropping, registration, segmentation and correction.

It should be understood that embodiments described in this part are neither intended to identify key or important features of embodiments of the present disclosure, nor construed to limit embodiments of the present disclosure. The additional features of the embodiments of the present disclosure will become more readily appreciated from the following descriptions.

### BRIEF DESCRIPTION OF THE DRAWINGS

The accompanying drawings are used for a better understanding of embodiments of the present disclosure and shall not construed to limit the present disclosure, in which:

FIG. 1 is a flowchart diagram showing a method for obtaining spatial omics single cell data according to an embodiment of the present disclosure;

FIG. 2 is a schematic diagram showing a workflow of the method for obtaining spatial omics single cell data according to an embodiment of the present disclosure;

FIG. 3 is a flowchart diagram showing a method for obtaining a stained image of a biological sample according to an embodiment of the present disclosure;

FIG. 4 is a flowchart diagram showing a method for stitching a plurality of sub-stained images according to an embodiment of the present disclosure;

FIG. 5 is a flowchart diagram showing another method for stitching a plurality of sub-stained images according to an embodiment of the present disclosure;

FIG. 6 is a schematic diagram showing a spatial distribution of a plurality of field of view according to an embodiment of the present disclosure;

FIG. 7 is a schematic diagram showing a pre-stitched result of a method for image pre-stitching according to an embodiment of the present disclosure;

FIG. 8a is a schematic diagram of a positional relationship between two stained images row-wise adjacent to each other, obtained by scanning in the horizontal direction during the pre-stitching according to an embodiment of the present disclosure;

FIG. 8b is a schematic diagram of a positional relationship between two stained images column-wise adjacent to each other, obtained by scanning in the vertical direction during the pre-stitching according to an embodiment of the present disclosure;

FIG. 9 is a schematic diagram of a single field-of-view template for stitching the biological sample according to an embodiment of the present disclosure;

FIG. 10 is a schematic diagram of a process for constructing the single field-of-view template for stitching the biological sample according to an embodiment of the present disclosure;

FIG. 11 is a flowchart diagram showing a method for performing image registration for the stained image and a visualization image of gene expression according to an embodiment of the present disclosure;

FIG. 12 shows a schematic diagram of a stained image according to an embodiment of the present disclosure;

FIG. 13 shows a schematic diagram of a third reference image according to an embodiment of the present disclosure;

FIG. 14a shows a schematic diagram of a third reference image with a plurality of periods according to an embodiment of the present disclosure;

FIG. 14b shows a schematic diagram of a visualization image of gene expression with a plurality of periods according to an embodiment of the present disclosure;

FIG. 15 is a schematic diagram of a predetermined period centered on a first offset according to an embodiment of the present disclosure;

FIG. 16 shows a schematic flow diagram of another method for performing image registration for the stained image and a visualization image of gene expression according to an embodiment of the present disclosure;

FIG. 17a shows a schematic diagram of a candidate image according to an embodiment of the present disclosure;

FIG. 17b shows a schematic diagram of an initial binary image according to an embodiment of the present disclosure;

FIG. 17c shows a schematic diagram of a target binary image according to an embodiment of the present disclosure;

FIG. 18 shows a schematic flow diagram of another method for performing image registration for the stained image and a visualization image of gene expression according to an embodiment of the present disclosure;

FIG. 19 shows a flowchart of determining image data in a method of a tissue segmentation of a sample image according to an embodiment of the present disclosure;

FIG. 20 shows a schematic diagram of the image data in the method of a tissue segmentation of a sample image according to an embodiment of the present disclosure;

FIG. 21 is a schematic diagram of a 2d convolution in the method of a tissue segmentation of a sample image according to an embodiment of the present disclosure;

FIG. 22 shows a schematic diagram of a low-level feature in the method of a tissue segmentation of a sample image according to an embodiment of the present disclosure;

FIG. 23 is a schematic flow diagram of a method of calculating a quality of a grayscale image corresponding to a tissue mask image according to an embodiment of the present disclosure;

FIG. 24 shows a structural schematic diagram of a relationship between an outer boundary image and an inner boundary image according to an embodiment of the present disclosure;

FIG. 25 shows a structural schematic diagram of another relationship between an outer boundary image and an inner boundary image according to an embodiment of the present disclosure;

FIG. 26 is a schematic flow diagram of a method of performing a cell segmentation for a registered image according to an embodiment of the present disclosure;

FIG. 27 shows a diagram of a segmentation performance of an image segmentation model on a mouse brain image according to an embodiment of the present disclosure;

FIG. 28 shows a schematic flow diagram of another method for performing a cell segmentation for a registered image according to an embodiment of the present disclosure;

FIG. 29 shows a schematic flow diagram of another method of cell segmentation processing according to an embodiment of the present disclosure;

FIG. 30 shows a schematic flow diagram of an example of the method according to an embodiment of the present disclosure;

FIG. 31 shows an exemplary diagram of an effect of an image of a gene expression according to an embodiment of the present disclosure;

FIG. 32 shows an exemplary diagram of an effect of a sharpened image obtained by a sharpening processing according to an embodiment of the present disclosure;

FIG. 33 shows a schematic diagram of an effect of an initial mask image according to an embodiment of the present disclosure;

FIG. 34 shows a schematic diagram of an effect of an outputting mask image according to an embodiment of the present disclosure;

FIG. 35 shows a schematic flow diagram of another method for performing a cell segmentation for a registered image according to an embodiment of the present disclosure;

FIG. 36 shows a schematic flow diagram of a method for determining cell-belonging for a background gene molecule unknown of the cell-belonging in the visualization image of gene expression according to an embodiment of the present disclosure;

FIG. 37 shows a schematic diagram of a correspondence between a DNB and a cell membrane according to an embodiment of the present disclosure;

FIG. 38 shows a schematic diagram of a correction result of correcting a background DNB according to an embodiment of the present disclosure;

FIG. 39 is a schematic flow diagram of another method for determining cell-belonging for a background gene molecule unknown of the cell-belonging in the visualization image of gene expression according to an embodiment of the present disclosure;

FIG. 40 is a schematic flow diagram of another method for determining cell-belonging for a background gene molecule unknown of the cell-belonging in the visualization image of gene expression according to an embodiment of the present disclosure;

FIG. 41 shows a schematic diagram of a center point of a target cell according to an embodiment of the present disclosure;

FIG. 42 is a schematic flow diagram of another method for determining cell-belonging for a background gene molecule unknown of the cell-belonging in the visualization image of gene expression according to an embodiment of the present disclosure;

FIG. 43 shows a schematic flow diagram of a method for determining coordinates of the point in the stained image according to an embodiment of the present disclosure;

FIG. 44 shows a schematic diagram of the stained image according to an embodiment of the present disclosure;

FIG. 45 shows a schematic flow diagram of a directed object detection method according to an embodiment of the present disclosure;

FIG. 46 is a schematic flow diagram of a method for obtaining gene expression information of a cell of the biological sample according to an embodiment of the present disclosure;

FIG. 47 shows a schematic structural diagram of a device for obtaining spatial omics single cell data according to an embodiment of the present disclosure;

FIG. 48 shows a schematic structural diagram of another device for obtaining spatial omics single cell data according to an embodiment of the present disclosure;

FIG. 49 shows a schematic frame diagram of an exemplary electronic device according to an embodiment of the present disclosure.

### DETAILED DESCRIPTION

The exemplary embodiments of the present disclosure will be illustrated in conjunction with the accompanying drawings. Various details of the embodiments of the present disclosure are included for better understanding, and they should be considered to be illustrative only. Therefore, various variants and modifications can be made to the embodiments of the present disclosure described herein, without departing from the spirit and scope of the present disclosure. Likewise, descriptions of well-known functions and structures are omitted from the following description for clarity and conciseness.

A method for obtaining spatial omics single cell data, and a device, an electronic device, and a storage medium thereof provide in embodiments of the present disclosure will be described below with reference to the drawings.

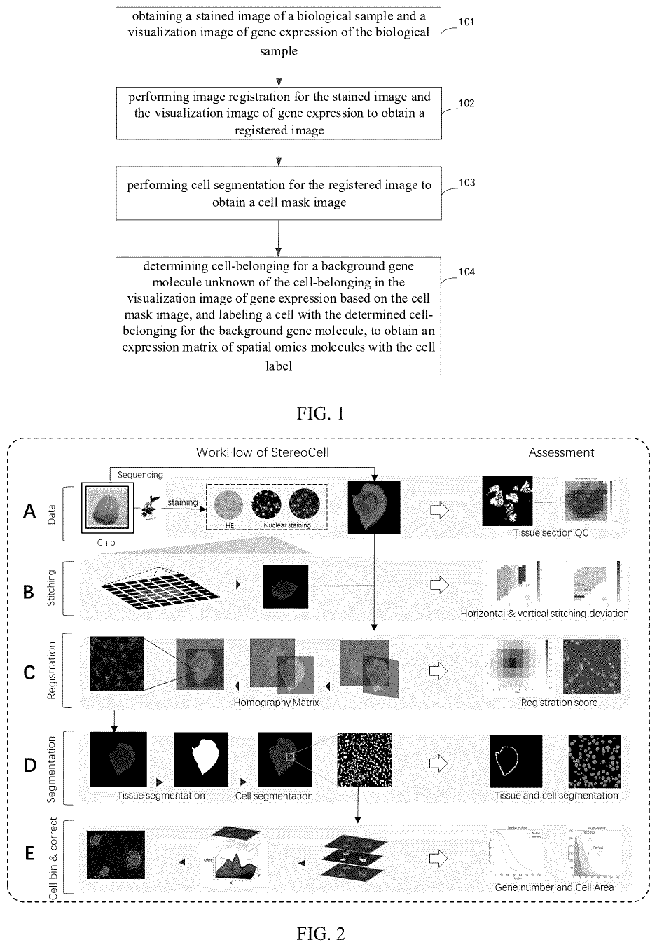

FIG. 1 is a flowchart diagram showing a method for obtaining spatial omics single cell data according to an embodiment of the present disclosure.

As shown in FIG. 1, the method includes steps as follows.

Step 101, obtaining a stained image of a biological sample and a visualization image of gene expression of the biological sample.

Based on Spatial resolved technology (SOP), the biological sample is stained and then the stained biological sample on a spatial-temporal chip is filmed with a microscope, so that the stained image is obtained.

As a feasible means of embodiments of the present disclosure, the staining may include but be not limited to the following techniques, e.g., Hematoxylin and Eosin Staining (HE), ssDNA Staining, and DAPI Staining, and is not limited specifically in embodiments of the present disclosure.

The obtained microscope film (the stained image) is generally in a tif format, including but not limited to 16 bit or 8 bit, and is available as both color and grayscale images, and is not limited specifically in embodiments of the present disclosure.

Being performed a spatial sequencing, and standard processing such as sequence alignment analysis, the gene expression matrix of the biological samples is obtained and converted into a visualization image of the gene expression.

The spatial-temporal chip is provided with regularly arranged sites for capturing genes of biological samples. Specifically, each site may capture one gene sequence of the biological sample, and the captured gene sequence or the base number thereof on each site may be determined by gene sequencing. The base number of the captured gene sequence on each site is then assigned as a value of an element of the gene expression matrix, so that the element of the gene expression matrix is corresponded with the site on the spatial-temporal chip individually, e.g. the element located on the first row and the first column of the gene expression matrix is corresponded with the site located on the upper left corner of the spatial-temporal chip, to obtain the obtain the gene expression matrix. The visualization image of the gene expression is then mapped based on the gene expression matrix. The pixel in the visualization image of the gene expression corresponds to the element of the gene expression matrix, and the pixel value of the pixel corresponds to the value of the element, so that the spatial information of the same biological sample is contained in the visualization image of the gene expression.

Methods of alignment analysis for sequenced sequences can be found in the related art for reference, and will not be repeated here in the embodiments of the present disclosure.

The step of obtaining a visualization image of gene expression of the biological sample includes, but is not limited to, the following manner: being performed a spatial sequencing, and sequence processing, the gene expression matrix of the biological samples is obtained and converted into the visualization image of the gene expression.

Step 102, performing image registration for the stained image and the visualization image of gene expression to obtain a registered image.

In some embodiments, the registration is performed based on line features in a stitched stained image.

Alternatively, in some embodiments, accurate and automatic registration with an accuracy at pixel level is performed by coordinate calculations using track crosses on the image and using tissue boundary for reference.

Alternatively, in some embodiments, the registration is performed by a tissue mask of the stitched stained image, and is not limited specifically in embodiments of the present disclosure.

Step 103, performing cell segmentation for the registered image to obtain a cell mask image.

The registered image is inputted into an image segmentation model to output a cell mask image containing a cell nucleus.

As a feasible means of embodiments of the present disclosure, before performing the cell segmentation, tissue segmentation can also be performed, and after the tissue segmentation, it is possible to focus only on the tissue region, ignoring the region outside the tissue, which improves the signal-to-noise ratio of the sample data.

Step 104, determining cell-belonging for a background gene molecule unknown of the cell-belonging in the visualization image of gene expression based on the cell mask image, and labeling a cell with the determined cell-belonging for the background gene molecule, to obtain an expression matrix of spatial omics molecules with the cell label.

After the cell segmentation performed in step 103, at least one cell nucleus is contained in a cell, however there are cases that some background gene molecules are not labeled as a cell.

Reassignment is performed for the background gene on the position of DNA nano-ball (DNB), which is not assigned to a cell. A certain area (e.g. 100*100 pixels) is circled around the position, and for each of the cells contained therein, a Gaussian mixture model is fitted, which assumes that N cells contained therein, then for that position there will be N probability values, and the cell with the largest probability is selected as the cell to which the position belongs. The above operation is performed for each position not assigned to a cell but with gene expression. With a secondary correction, the signal-to-noise ratio and the accuracy of cell segmentation and registration can be improved.

In practice, in order to prevent over-allocation, a probability threshold is set for selecting and labeling the cell with the largest probability as the cell with the cell-belonging, so that when the probability is lower than the threshold, the cell with the cell-belonging is not labeled. The probability threshold is not explicitly limited.

FIG. 1 has illustrated the method for obtaining spatial omics single cell data, referring to FIG. 2, a schematic diagram showing a workflow of the method for obtaining spatial omics single cell data according to an embodiment of the present disclosure. A mouse brain section is illustrated as an example in the schematic diagram, wherein A corresponds to step 101, C corresponds to step 102, D corresponds to step 103, and E corresponds to step 104.

Obtaining a stained image of a biological sample and a visualization image of gene expression of the biological sample; performing image registration for the stained image and the visualization image of gene expression to obtain a registered image; performing cell segmentation for the registered image to obtain a cell mask image; and determining cell-belonging for a background gene molecule unknown of the cell-belonging in the visualization image of gene expression based on the cell mask image, and labeling a cell with the determined cell-belonging for the background gene molecule, to obtain an expression matrix of spatial omics molecules with the cell label. Compared with the prior art, the present disclosure obtains high-precision spatial single cell data by means of the image of gene expression matrix, and a processing flow involving stitching, registration, segmentation and correction.

As a feasible means of embodiments of the present disclosure, said obtaining a stained image of a biological sample in step 101 may be performed by, but be not limited to, the following three modes:

Mode 1, obtaining the stained image of the biological sample with a microscopy.

Mode 2, obtaining a stitched stained image of the biological sample with a microscope, wherein the stitched stained image is stitched from a plurality of sub-stained images.

Mode 3, obtaining a plurality of sub-stained images of the biological sample with a microscopy, and stitching the plurality of sub-stained images to obtain the stitched stained image.

Among them, Mode 1 and Mode 2 are the results of performing stitching under the microscope, Mode 1 is to obtain a large image (stained image) directly through the microscope; Mode 2 is to obtain a stained image by stitching multiple sub-stained images under the microscope after obtaining multiple small images (sub-stained images); Mode 3 is to obtain a stitching result of the stained image by stitching with a preset algorithm, when the stitching results under microscope do not meet the needs. In the specific embodiments of the present disclosure, the mode of obtaining the stained images is not limited.

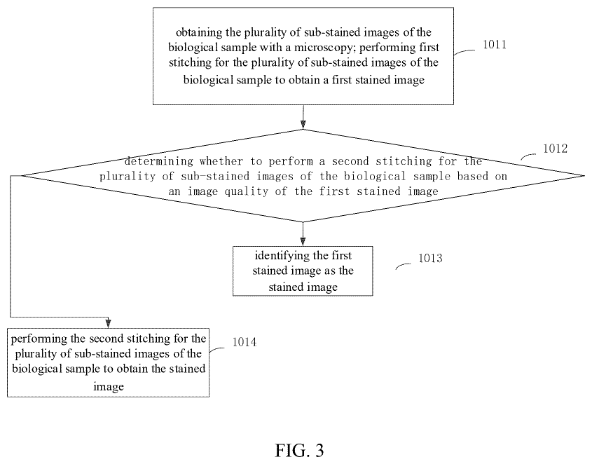

In some embodiments, as shown in FIG. 3, a flowchart diagram showing a method for obtaining a stained image of a biological sample according to an embodiment of the present disclosure, the method includes steps as follows.

Step 1011, obtaining the plurality of sub-stained images of the biological sample with a microscopy; and performing first stitching for the plurality of sub-stained images of the biological sample to obtain a first stained image.

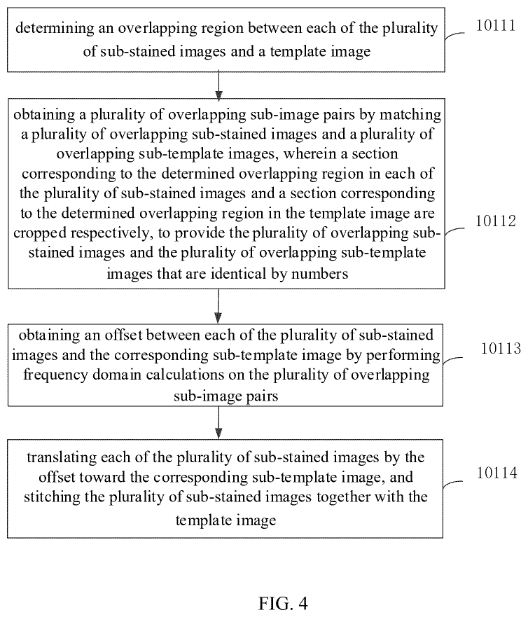

The stitching may be performed in, but is not limited to, the following modes as shown in FIG. 4, including:

Step 10111, determining an overlapping region between each of the plurality of sub-stained images and a template image.

In some embodiments, said determining an overlapping region between each of the plurality of sub-stained images and a template image may include determining a matching direction of the sub-stained image and the template image; and selecting adjacent regions of the same size in the sub-stained image and the template image to serve as the overlap region in the determined matching direction.

For example, the matching direction (or stitching direction) may be determined based on the direction of movement of the capturing device when the two images are captured. If the sub-stained image and the template image are two images sequentially captured by the capturing device while moving horizontally, the matching direction between those two images may be determined as a horizontal direction. Other available modes may be used to determine the relative positional relationship between the two images and thus determine the matching direction, will not be repeated here.

In some embodiments, the overlapping region is selected such that a ratio of actual overlapping portions between the sub-stained image and the template image in said overlapping region is greater than a predetermined threshold.

In some embodiments, this predetermined threshold is determined to be 10%, or 30%, etc. It should be noted that this value is only an illustrative value being used. In some other embodiments, other values that are larger or smaller may also be taken as the predetermined threshold.

Please continue to refer to FIG. 2, the step corresponds to step B in FIG. 2.

Step 10112, obtaining a plurality of overlapping sub-image pairs by matching a plurality of overlapping sub-stained images and a plurality of overlapping sub-template images, wherein a section corresponding to the determined overlapping region in each of the plurality of sub-stained images and a section corresponding to the determined overlapping region in the template image are cropped respectively, to provide the plurality of overlapping sub-stained images and the plurality of overlapping sub-template images that are identical by numbers.

In some embodiments, the operation may include: performing denoising and/or windowing for the sections of the sub-stained images and the template images identified as overlapping regions, respectively; merging the sub-stained images and the template images, subjected to the denoising and/or windowing, in a direction perpendicular to the matching direction; and resampling sections of the sub-stained images and the template images, subjected to the denoising and/or windowing, at the same image cropping size, respectively, so that obtaining the plurality of overlapping sub-stained images and the plurality of overlapping sub-template images; and sequentially matching the plurality of overlapping sub-stained images and the plurality of overlapping sub-template images one by one in a direction perpendicular to the matching direction to form the plurality of overlapping sub-image pairs.

The denoising in the above operation may be performed with, for example, Gaussian filtering, but other denoising methods used in the field of image processing may also be used. The windowing may be used for obtaining an image with suppressed spectral leakage. The windows used may include, but be not limited to, Hanning windows, Hamming windows, and the like.

In some embodiments of the present disclosure, the resampling described above may be performed with an approach of sliding window. The approach of sliding window may set a certain overlapping region, and the plurality of stacked small sub-images correspondingly ensure that the cropping regions of the source image and the template image are located in the overlapping region and in the same position in a coordinate system. The resampling may be implemented by any available means, such as cropping more regions of features using a object detection method, etc., without being limited to the approach of sliding window described above.

After obtaining the plurality of overlapping sub-image pairs, the method shown in FIG. 1 may optionally include removing overlapping sub-image pairs that have fewer features than a particular criterion and/or more noise than a particular criterion from said plurality of overlapping sub-image pairs. For example, sub-image pairs with fewer features and/or more noise may be filtered using gradient detection. The gradient detection methods used include, but are not limited to, methods such as Sobel, Canny, and the like. In addition, any other method that can be used to detect a feature number and/or noise in an image may be used herein without being limited to gradient detection methods.

Step 10113, obtaining an offset between each of the plurality of sub-stained images and the corresponding sub-template image by performing frequency domain calculations on the plurality of overlapping sub-image pairs; and

Optionally, the overlapping sub-image pairs for which the superposition and resonance of the frequency domain is performed herein are the overlapping sub-image pairs obtained by removing the sub-image pairs with fewer features and/or more noise in the optional step described above.

In some embodiments, the step may be performed using a Dirac function. For example, the step includes: stacking the mutual power spectrum for the plurality of overlapping sub-image pairs and obtaining an offset from the stacked mutual power spectrum using the Dirac function.

In some specific embodiments, each of the plurality of overlapping sub-image pairs may be transformed from a position coordinate system to a frequency domain. This transformation may be performed, for example, by a two-dimensional discrete Fourier transform, or by any other approach that enables a domain transformation, which will not be described herein. The mutual power spectrum corresponding to each overlapping sub-image pair of the plurality of overlapping sub-image pairs may then be calculated. Subjecting the obtained mutual power spectrum corresponding to each overlapping sub-image pair of the plurality of overlapping sub-image pairs to a discrete Fourier inverse transformation so that obtaining a Dirac function representation of the overlapping region. The coordinate, at which the maximum peak of the obtained Dirac function representation of the overlapping region is located, is determined as the offset.

In some embodiments, before accumulating the mutual power spectrum corresponding to each of the obtained overlapping sub-image pairs, for each of the obtained overlapping sub-image pairs, features in that overlapping sub-image pair may also be improved to be more significant. For example, the overlapping sub-image pair may be transformed to a position coordinate system and then subjected to a windowing process so that enhancing the features in that overlapping sub-image. The windowing function used herein may be a Hanning window or a Hamming window Hamming window, or any other function that may enhance features in the image. Enhancement of the features in the overlapping sub-images may effectively improve the success rate of image registration for images with insignificant features. Herein, the approach of enhancing the features in the image used may not be limited to the windowing process, but may be any other approach capable of enhancing the image features.

In some embodiments, said subjecting the obtained mutual power spectrum corresponding to each overlapping sub-image pair of the plurality of overlapping sub-image pairs to a discrete Fourier inverse transformation so that obtaining a Dirac function representation of the overlapping region may include: subjecting the obtained mutual power spectrum corresponding to each overlapping sub-image pair of the plurality of overlapping sub-image pairs to a discrete Fourier inverse transformation so that obtaining a Dirac function representation of the stack; and performing a weighted cumulative sum for the obtained Dirac function representation of the stack to obtain a Dirac function representation of the overlapping region. In this embodiment, overlapping sub-image pairs with more significant features may be assigned larger weighting coefficient. For example, the weighting coefficient for each overlapping sub-image pair may depend on the energy spectrum of the overlapping sub-image pair (e.g., the energy intensity of the maximum peak of the Dirac function) and/or the matching number of the feature points of the overlapping sub-image pair and/or the image clarity of the overlapping sub-image pair, or may be based on any other metric capable of characterizing whether or not the overlapping sub-image pair has a more significant feature.

Step 10114, translating each of the plurality of sub-stained images by the offset toward the corresponding sub-template image, and stitching the plurality of sub-stained images together with the template image.

Step 1012, determining whether to perform a second stitching for the plurality of sub-stained images of the biological sample based on an image quality of the first stained image.

In some embodiments, the image quality of the first stained image is set based on the needs for obtaining the spatial omics single cell data, which are not limited by the embodiments of the present disclosure.

If no, step 1013 is performed; if yes, step 1014 is performed.

Step 1013, identifying the first stained image as the stained image.

Step 1014, performing the second stitching for the plurality of sub-stained images of the biological sample to obtain the stained image.

The specific implementation process of the second splicing may be referred to the relevant description of FIG. 4, and will not be repeated here.

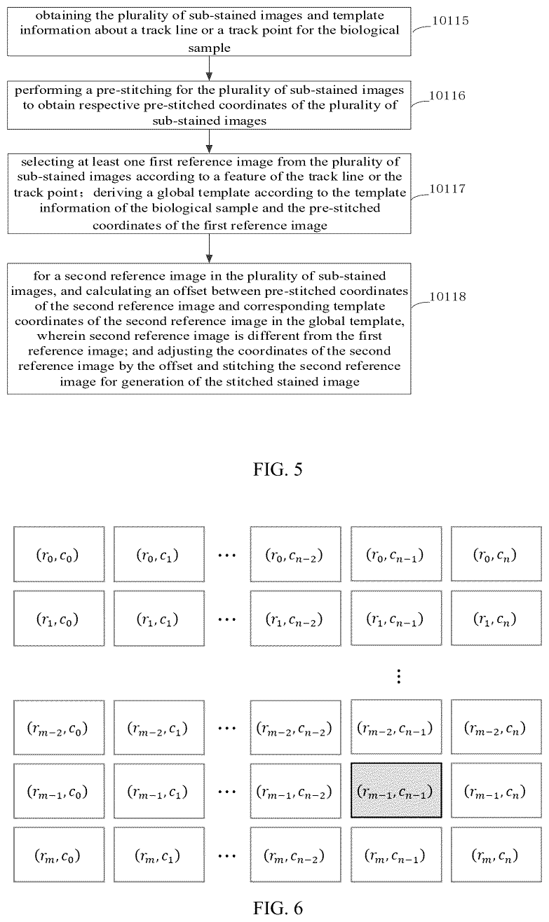

As shown in FIG. 5, a flowchart of another method for stitching a plurality of sub-stained images to obtain a stitched stained image according to an embodiment of the present disclosure, the method includes steps as follows.

Step 10115, obtaining the plurality of sub-stained images and template information about a track line or a track cross for the biological sample.

During the sequencing process, a number of images can be taken sequentially for local regions of a single biological sample. A number of track lines are distributed on the image of each field of view in the horizontal and vertical directions, and the intersection points of the track lines are the track crosses (or “track cross”). The track lines and the track crosses can be converted into each other; the track crosses can be obtained according to the two track lines that cross; the track lines can be obtained according to the track crosses that are co-located. Said template information may include an index number of a plurality of track lines on said biological sample. As known to those skilled in the art, the distribution pattern of the track lines on the biological sample is specified at the beginning of the design of the biological sample. For example, assuming that there are 10 track lines in the vertical direction of the biological sample, and the spacing between them may be, for example, 1, 2, 3, 4, 5, 4, 3, 2, 1, respectively. Assuming that the position, at which a first track line is located, is the starting point (with the coordinate of 0), the coordinates of the horizontal axis (x-axis) of the above 10 track lines are 0, 1, 3, 6, 10, 15, 19, 22, 24, 25, respectively. The index numbers in the template information of the biological samples can be associated with the arrangement of the track lines in the biological samples, and the index numbers of the above 10 track lines can be artificially stipulated to be, for example, 0, 1, 2, 3, 4, 5, 6, 7, 8, and 0, respectively.

In addition, the obtained template information of the biological sample may also include periodicity. For example, for the 10 track lines in the vertical direction of the biological sample described above, they may be extended one or more periods to the left and to the right, respectively. For example, after being extended by each periods to the left and right, the spacing between the track lines formed are 1, 2, 3, 4, 5, 4, 3, 2, 1, 1, 2, 3, 4, 5, 4, 3, 2, 1, 1, 2, 3, 4, 5, 4, 3, 2, 1, respectively, which results in the coordinates of the horizontal axes (x-axis) of the expanded 28 track lines being −25, −24, −22, −19, −15, −10, −6, −3, −1, 0, 1, 3, 6, 10, 15, 19, 22, 24, 25, 26, 28, 31, 35, 40, 44, 47, 49, 50, respectively, and their corresponding index numbers being 0, 1, 2, 3, 4, 5, 6, 7, 8, 0, 1, 2, 3, 4, 5, 6, 7, 8, 0, 1, 2, 3, 4, 5, 6, 7, 8, 0, 1, 2, 3, 4, 5, 6, 7, 8, 0, respectively.

It should be noted that, generally, a given track line corresponds to a unique index number in a biological sample, whereas a given index number of a biological sample corresponds to multiple track lines, which is determined by the periodicity of the design of track lines on the biological sample.

The microscope may take images of the biological sample, for example by scanning the biological sample row by row or column by column. The spatial distribution of the plurality of images after scanning may be as illustrated in FIG. 6, said plurality of images may be provided as a total of, for example, n+1 columns, m+1 rows, including an image on the first row and first column (r0, c0) to an image on the last row and last column (rm, cn). For the image (rm-1, cn-1) as illustrated in FIG. 3, up to 8 adjacent images may be provided for this image. In addition, the microscope may record the camera position information when taking an image, and subsequently based on any camera position information recorded when taking the image, the position corresponding to the image may be determined and the adjacent images thereof may be determined for the image.

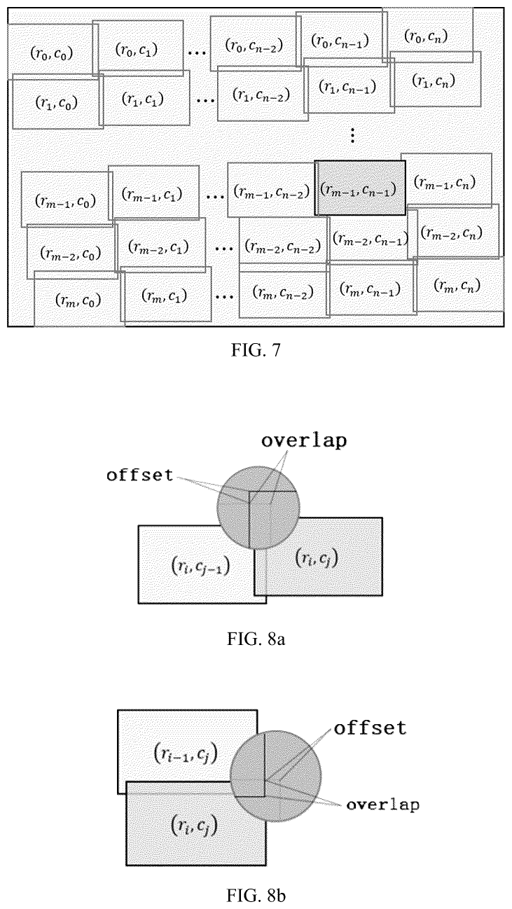

FIG. 7 is a schematic diagram showing a pre-stitched result of a method for image pre-stitching according to an embodiment of the present disclosure.

Step 10116, performing a pre-stitching for the plurality of sub-stained images to obtain respective pre-stitched coordinates of the plurality of sub-stained images.

The plurality of stained images may be scanned, and the left adjacent image and the right adjacent image for the current image may be determined using, for example, a 2-nearest neighbor approach based on the scanning sequence or the camera position information when taking the image, and further the offset and overlap between the stained image and the left adjacent image as well as the right adjacent image may be determined. During the scanning process, the offset and the overlap between the adjacent stained images may be obtained, preferably using a fast Fourier transform (FFT) or a scale invariant feature transform (SIFT) in combination with the template information of the biological sample. The pre-stitched coordinates of the plurality of stained images may be obtained according to the offset and the overlap between the adjacent stained images determined during the image scanning process.

FIG. 8a is a schematic diagram of a positional relationship between two stained images row-wise adjacent to each other, obtained by scanning in the horizontal direction during the pre-stitching; FIG. 8b is a schematic diagram of a positional relationship between two stained images column-wise adjacent to each other, obtained by scanning in the vertical direction during the pre-stitching.

Specifically, FIG. 8a and FIG. 8b illustrate the offset and the overlap between the adjacent stained images obtained after scanning the stained images in the horizontal direction and the vertical direction, respectively.

For the scanning in the horizontal direction (refer to FIG. 8a), the overlapping distance between two stained images row-wise adjacent to each other in the horizontal direction is referred to as the overlap in the horizontal direction, and the deviating distance between them in the vertical direction is referred to as the offset in the vertical direction. Similarly, for the scanning in the vertical direction (refer to FIG. 8b), the obtained overlapping distance between two stained images column-wise adjacent to each other in the vertical direction is referred to as the overlap in the vertical direction, and the deviating distance between them in the horizontal direction is referred to as the offset in the horizontal direction. With the scanning in the horizontal direction and the vertical direction respectively, the offset matrix A and the overlap matrix B of the plurality of stained images corresponding to the respective direction may be obtained. Using the offset matrix A and the overlap matrix B, the spatial coordinates of the individual stained images may be mapped, so as to obtain the pre-stitched image of the plurality of stained images as shown in FIG. 7.

However, a pixel-level accuracy nay not be arrived at by the pre-stitching for the stained image alone, so accurate adjustments for the stained image in the steps S10117 and S10118 are further needed on this basis.

Step 10117, selecting at least one first reference image from the plurality of sub-stained images according to a feature of the track line or the track cross; deriving a global template according to the template information of the biological sample and the pre-stitched coordinates of the first reference image.

Said first reference image (or a reference image) selected may be the image with the most significant track line features or the image with the most track crosses among said plurality of stained images. Said first reference image is, for example, the image (rm-1, cn-1) highlighted in FIG. 6 and FIG. 7, and then the selected image (rm-1, cn-1) is subjected to track line detection, and the spacing between the detected adjacent track lines is matched with the spacing between the track lines specified during the design of the chip referred to above, so as to determine the index number corresponding to the track line in the selected first reference image. In other words, the index number corresponding to each track line in said first reference image may be determined based on the distance between adjacent track lines in said first reference image. Then, a global template may be derived based on said index number of said first reference image and the pre-stitched coordinates of said first reference image.

In some embodiments, since the track lines may be covered a tissue section of the biological sample and some of the track crosses may be visible through the tissue cavity, therefore, the probability of a complete track cross visible is higher than that of a complete track line visible within an image, and thus the image with the most track crosses may also be preferably selected as the first reference image.

In some embodiments, the index number(s) corresponding to each track line in said first reference image is determined based on a distance between adjacent track lines in said first reference image, and a global template is derived based on said index number(s) of said first reference image and the pre-stitched coordinates of said first reference image.

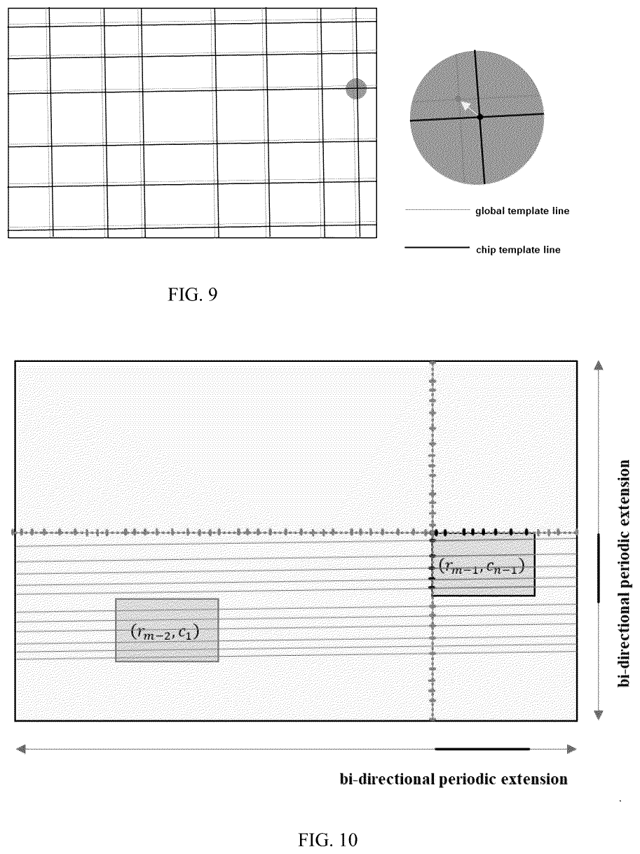

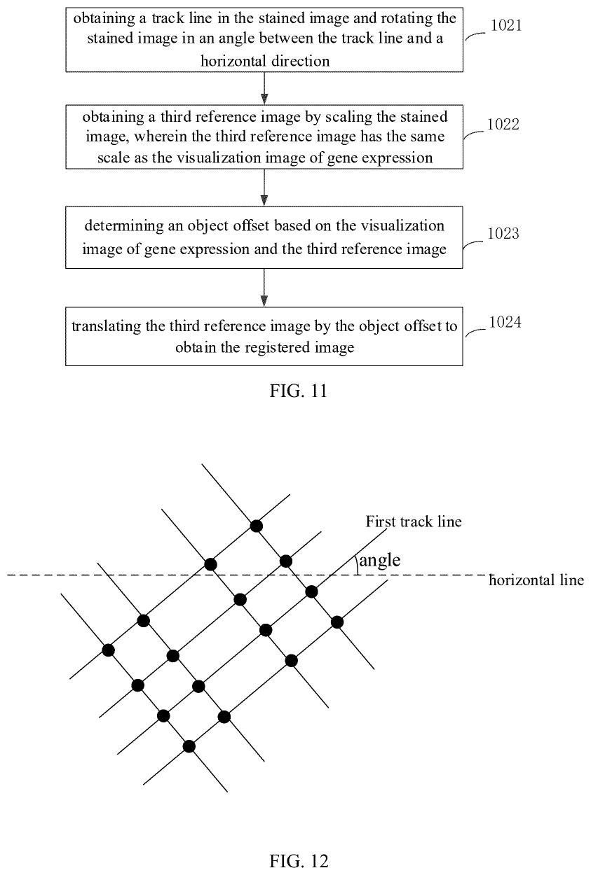

FIG. 9 is a schematic diagram illustrating a global template for stitching the biological sample according to an embodiment of the present disclosure; and FIG. 10 is a schematic diagram of a process for constructing the global template for stitching the biological sample according to an embodiment of the present disclosure.

Referring to the example of constructing the global template shown in FIG. 10, based on the position coordinates of the selected first reference image (rm-1, cn-1) in the pre-stitching stage and the index numbers of the track lines on the selected first reference image (rm-1, cn-1), the global template (or global reference system) based on the first reference image (rm-1, cn-1) may be obtained by carrying out a left-right bi-directional periodic extension in the horizontal direction and an up-down bi-directional periodic extension in the vertical direction.

The adjustment for the coordinates of the second reference image being different from the selected first reference image (rm-1, cn-1) may be classified into two categories, i.e., the adjustment for the coordinates of the second reference image containing the track crosses and the adjustment for the coordinates of the second reference image without the track crosses.

The adjustment for the coordinates of the second reference image containing the track crosses is performed by adding respective unique offsets of the second reference image containing the track crosses to the pre-stitched coordinates of each said second reference image containing the track crosses. The offset of the second reference image is a vector formed by pointing from a track cross on the second reference image to a template point in the global template corresponding to the track cross on the second reference image. Reference may be made to the offset shown with arrows in the locally enlarged schematic diagram of FIG. 9, the offset represents the vector formed by pointing from a track cross on a biological sample to a track cross of template line on the derived global template corresponding to the track cross on the biological sample.

Step 10118, for the second reference image in the plurality of sub-stained images, calculating the offset between the pre-stitched coordinates of the second reference image and corresponding template coordinates of the second reference image in the global template, wherein second reference image is different from the first reference image, and adjusting the coordinates of the second reference image by the offset and stitching the second reference image for generation of the stitched stained image.

A respective offset for each track cross in each said second reference image containing the track crosses is obtained; and for each said second reference image, the offset with a median angle or a median distance among said offsets is selected as the unique offset for the second reference image.

Alternatively, the respective offset for each track cross in each said second reference image containing the track crosses is obtained; and said offsets are ranked according to the vector length of the offset; the offset at a predetermined ranking position is selected as the unique offset for that second reference image.

Adjusted coordinates of the second reference image containing the track cross may be obtained by adding the unique offset for each said second reference image containing the track cross to the respective offset for said second reference image containing the track cross.

The adjustment for the coordinates of the second reference image without the track crosses may be performed using a nearest neighbor adjustment method, which including: for each said second reference image without the track crosses, adjustment for coordinate is performed according to the unique offset of the second reference image containing track crosses that is nearest to the second reference image without the track crosses, and the offset and the overlap between the nearest second reference image containing track crosses and the second reference image without the track crosses.

Specifically, due to issues such as covering of tissue, contamination of chip features, and capture quality of the micro-camera, there are images without track crosses thereon, in this case, the second reference image without track crosses may be aligned based on the offset and overlap between the second reference image without track crosses and the adjacent second reference image or the nearest second reference image containing track crosses adjusted. It should be noted that the nearest distance is measured by a Manhattan distance, not a Euclidean distance.

The adjustment for the coordinates of the second reference image further includes performing a seam fusion processing for the neighboring second reference images, e.g., a linear weighted fusion method, etc., may be used.

In some embodiments, as shown in FIG. 11, a flowchart diagram showing a method for performing image registration for the stained image and a visualization image of gene expression according to an embodiment of the present disclosure, the method includes:

Step 1021, obtaining a track line in the stained image and rotating the stained image in an angle between the track line and a horizontal direction, wherein the stained image and the visualization image of gene expression each include a plurality of track lines and/or a plurality of track crosses, two of the track lines intersect to form the track crosses, and each of the plurality of track lines is corresponded with an index number.

The track line of the microscope image is not always in the horizontal direction and the vertical direction because the stained image is obtained by microscope image filming, therefore the first track line in the microscope image may be selected as a reference so that an angle between the first track line and the horizontal direction may be calculated, enabling the microscope image to be located in the horizontal direction after being rotated by the said angle. Referring to FIG. 12, a schematic diagram of a stained image is shown. As shown in FIG. 12, the stained image is not located in the horizontal direction, and there exists an angle between the first track line and the horizontal direction. Therefore, said angle may be calculated and the stained image may be rotated to the horizontal direction.

Being manifested as multiple discrete points, the visualization image of gene expression is generated from a gene expression matrix, and exhibits an inadequate capability to express aggregating features or edge features of the biological sample.

In a preferable embodiment, tissue segmentation may be performed for the visualization image of gene expression to obtain a biological sample boundary manifested by the gene expression, and a convolutional enhancement processing may be performed on an approximate region, where the sample boundary located, to enhance the edge features of the visualization image of gene expression, and to facilitate the subsequent registration for the microscope image and the visualization image of gene expression.

Step 1022, obtaining a third reference image by scaling the stained image, wherein the third reference image has the same scale as the visualization image of gene expression.

When the stained image is an image filmed by a microscope, since the visualization image of gene expression may be used as a reference image, the scaling of the stained image filmed by the microscope and the visualization image of gene expression may be different. To perform the registration for the stained image with the visualization image of gene expression, the stained image needs to be scaled to obtain a third reference image having the same scale as the visualization image of gene expression.

In a possible embodiment, the scaling for the stained image may be determined based on a spacing between the track lines with index number in the stained image and corresponding index number in the visualization image of gene expression. Specifically, a second track line in the stained image is obtained; a first spacing is determined based on the first track line and the second track line of the stained image; a third track line and a fourth track line in the visualization image of gene expression are obtained, wherein the index number of the third track line is the same as the index number of the first track line, and the index number of the fourth track line is the same as the index number of the second track line; and a second spacing is determined based on the third track line and the fourth track line. Then a scaling value between the stained image and the visualization image of gene expression is determined based on the first spacing and the second spacing, and the stained image is enlarged or reduced in accordance with the aforementioned scaling value to obtain the third reference image having the same scale as the visualization image of gene expression. Wherein the first track line and the second track line are parallel, and the third track line and the fourth track line are parallel.

When determining the first spacing based on the first track line and the second track line, track crosses on the first track line and the track crosses on the second track line may be used for calculation. When two track crosses have the same horizontal or vertical coordinates, the difference of the corresponding vertical coordinates or the corresponding horizontal coordinates may be used as the first spacing. Similarly, the second spacing between the third track line and the fourth track line may be determined based on the same principle. It should be noted that the method for calculating the scaling of a stained image and the visualization image of gene expression provided in the above embodiments is only an exemplary illustration, and is not limited to the above embodiments.

Step 1023, determining an object offset based on the visualization image of gene expression and the third reference image. When the stained image is scaled to the third reference image having the same scale as the visualization image of gene expression, the object offset may be subsequently determined based on the visualization image of gene expression, and the third reference image.

As shown in the above embodiment, after the original stained image being rotated to the horizontal direction, there may be a difference of angle multiples of 90° between the stained image and the visualization image of gene expression. Therefore, to further improve the accuracy of the image processing, after the stained image being scaled to the third reference image having the same scale as the visualization image of gene expression, the third reference image may be rotated according to a predetermined rotation angle, and a corresponding object offset may be determined at each rotation angle. The following will be described in connection with a specific application scenario.

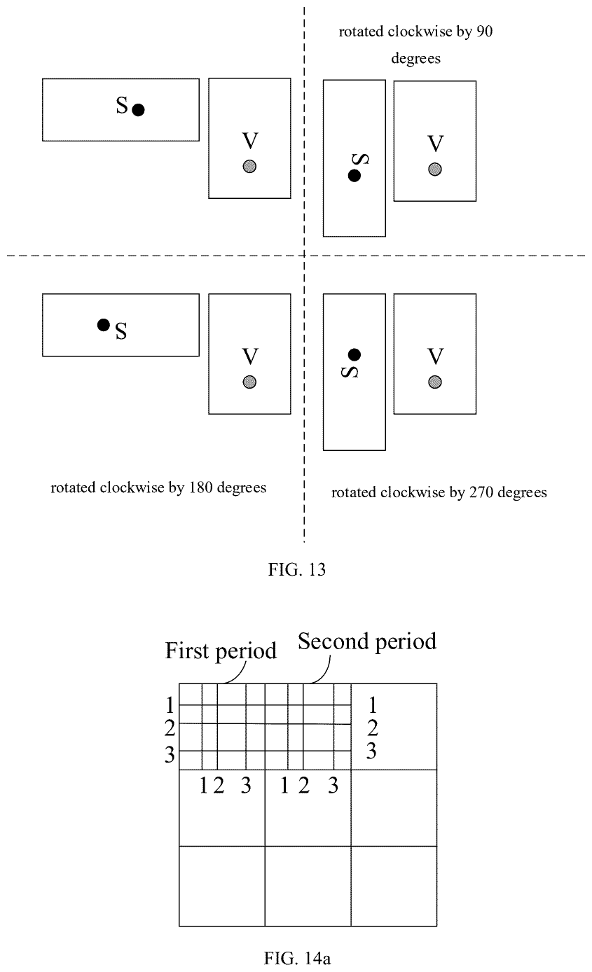

As shown in FIG. 13, S denotes the third reference image, V denotes the visualization image of gene expression, the black dot denotes the centroid of the third reference image, the gray dot denotes the centroid of the visualization image of gene expression. The diagrams of the four regions of FIG. 13 denote, respectively, the original third reference image and the images after rotating the third reference image clockwise by 90 degrees, 180 degrees, and 270 degrees. After rotating the third reference image, aligning may be performed according to the origin coordinates of the third reference image and the origin coordinates of the visualization image of gene expression, and then the object offset may be determined according to the origin-aligned third reference image and the visualization image of gene expression.

With respect to any one of the predetermined rotation angles, for more convenient and accurate image processing, the present embodiment provides a preferable implementation method, including firstly, preliminarily positioning the third reference image to approximately the same position of the visualization image of gene expression, based on the centroid coordinates of the third reference image and the visualization image of gene expression; and then performing a precise positioning by calculating the object offset based on the translated third reference image. In specific implementation, the stained image is scaled to obtain a fifth reference image, wherein the fifth reference image has the same scale as the visualization image of gene expression. Then a first centroid coordinates of the fifth reference image and a second centroid coordinate of the visualization image of gene expression are calculated respectively, and a centroid offset is determined based on the first centroid coordinate and the second centroid coordinate, and the fifth reference image is translated based on the centroid offset to obtain the fourth reference image, i.e., the fourth reference image for preliminary positioning is obtained. Wherein, the density centroid algorithm based on pixel grayscale values may be utilized to perform the calculation of the centroid coordinates of the fifth reference image and the visualization image of gene expression, and the present embodiment does not limit hereto.

It should be noted that during the image translation in the intermediate process, the processing may be performed on the temporary image of the replicated stained image, i.e., the stained image is not translated for registration. When the calculation is completed to obtain the final object offset, and then the stained image is translated at one time to be registered with the visualization image of gene expression.

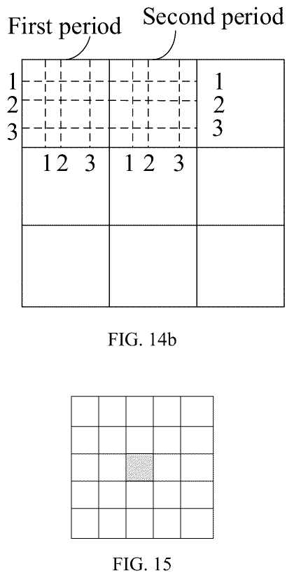

After obtaining the fourth reference image preliminarily positioned, the object offset may be determined based on the fourth reference image and the visualization image of gene expression. In practice, the track line that provided with the spatial-temporal chip may have a plurality of periods, and thus the microscope image and the visualization image of gene expression obtained based on the spatial-temporal chip may also have a plurality of periods, i.e., the fourth reference image and the visualization image of gene expression have a plurality of periods. Each period has a corresponding sub-image with the same number of track lines and track crosses in each sub-image, and the index numbers of the track lines between the sub-images are one-to-one corresponded. As shown in FIG. 14a and FIG. 14b, FIG. 14a illustrates a third reference image with a plurality of periods, and FIG. 14b illustrates a visualization image of gene expression with a plurality of periods. In FIG. 14a, only the sub-images of the first period and the second period are represented, and from which it can be learned that the sub-images of each period have the same number of index lines and index points, and each index line corresponds to an index number. The sub-images corresponding to the first period and the second period are also represented in FIG. 14b. The schematic representation of the third reference image and the visualization image of gene expression provided in the above embodiments is only a possible implementation and is not intended to be any limitation on the specific form of the image. Therefore, when determining the object offset based on the fourth reference image and the visualization image of gene expression, the effect of the periods needs to be considered.

For specific implementation, firstly, the first offset, being a pending offset between the fourth reference image and the visualization image of gene expression, may be determined based on the fourth reference image and the visualization image of gene expression; and then centering on the first offset, a plurality of second offsets corresponding to the first offset within a predetermined periods may be determined, i.e., each period within the predetermined periods corresponds to one second offset. For any one of the second offsets, a fourth reference image is translated based on the second offset to obtain a sixth reference image; and then a first similarity between the sixth reference image and the visualization image of gene expression is calculated, thereby obtaining a first similarity corresponding to each period within the predetermined periods. Since the above-determined similarity is obtained at any one of the rotation angles, it is necessary to re-execute the above steps at other predetermined rotation angles to obtain a plurality of first similarities corresponding to each rotation angle. Based on the plurality of first similarities determined for all rotation angles and all periods, a first maximum similarity is determined, and the sixth reference image corresponding to this first maximum similarity is determined as the third reference image. The second offset corresponding to the third reference image may then be determined as the object offset.

Wherein, when determining the first offset based on the fourth reference image and the visualization image of gene expression, one possible implementation is to obtain a plurality of groups of track crosses sets at first, wherein each group of track crosses sets contains a first track cross and a second track cross, wherein the first track cross is located in the fourth reference image and the second track cross is located in the visualization image of gene expression, and wherein the first track cross and the second track cross have the same index identifier. Since the track cross is formed by two intersecting track lines, each of which has an index number, the index identifier of the track cross may consist of the index numbers of the two intersecting track lines of the track cross. In this embodiment, since there is a plurality of track crosses in both the fourth reference image and the visualization image of gene expression, it is necessary to obtain track crosses having a corresponding relationship before they can be used as a group of track crosses sets, i.e., track crosses with the same index number. For any group of track crosses set in the plurality group of track crosses sets, a fourth offset may be determined based on the coordinates of the first track cross and the coordinates of the second track cross, and then the first offset can be determined based on the plurality of fourth offsets.

In order to facilitate a clearer understanding of the present solution, before describing the implementation of the third offset, the implementation of the fourth offset is first described.

In specific implementation, the visualization image of gene expression may have a plurality of periods, i.e., have a plurality of sub-images, thus the first track cross and the second track cross obtained with the same index identifier may not belong to the corresponding period, so there may be a plurality of the second track crosses determined. In determining the fourth offset based on the coordinates of the first track cross and the coordinates of the second track cross, any one of the first track crosses in the fourth reference image may be selected first, and a plurality of second track crosses having the same index identifiers may be determined based on the index identifiers of the first track crosses. In order to obtain the second track cross corresponding to the first track cross, the Euclidean distance between the first track cross and each of the second track crosses may be calculated respectively; and after obtaining the plurality of Euclidean distances by the calculation, the second track cross corresponding to the smallest Euclidean distance is obtained as the second track cross corresponding to the first track cross. The fourth offset is then determined according to the coordinate difference between the second track cross corresponding to the smallest Euclidean distance and the first track cross. Since the fourth reference image has a plurality of track crosses, which are corresponding to a plurality of groups of track crosses sets, it is necessary to iterate through all groups of track crosses sets to determine the fourth offset corresponding to each group of track crosses sets in accordance with the above-described method of determining the basic offset. In a possible implementation, the fourth offsets that are out of a predetermined range may be first excluded from the plurality of fourth offsets determined, and then an object offset is determined based on the remaining fourth offsets. For example, a median offset among the plurality of fourth offsets may be selected as the object offset, or an average of all the fourth offsets may be calculated as the object offset to maximize the accuracy of offset determination. It should be to be noted that the present embodiment does not limit the specific manner of determining the object offset based on the fourth offset.

Image translation during predetermined periods will be described below in relation to a specific application scenario.

Referring to FIG. 15, a schematic diagram of a predetermined period centered on a first offset.

In a possible implementation, the visualization image of gene expression is used as a reference. As shown in FIG. 15, the gray area in the middle indicates the period in which the second track cross is located, and after determining the first offset based on the first track cross and the second track cross, the second offset corresponding to each period is determined by selecting nearby 5*5 periods centered on this first offset (the period to which the corresponding second track cross belongs), and the difference between the second offsets corresponding to adjacent periods is the width of the sub-image, and so on, the second offsets corresponding to each period may be determined.

A sixth reference image may be obtained by translating the fourth reference image for the second offset corresponding to any one of periods, and a similarity between the sixth reference image and the visualization image of gene expression is calculated. In calculating the similarity, the present embodiment provides a possible implementation, in which a frequency domain transform is performed on the sixth reference image to obtain a first frequency domain image, a frequency domain transform is performed on the visualization image of gene expression to obtain a second frequency domain image, and a Hamming distance between the first frequency domain image and the second frequency domain image is calculated to serve as the similarity. When the Hamming distance is smaller, it indicates that the similarity between the first frequency domain image and the second frequency domain image is greater. After determining the Hamming distances corresponding to all the predetermined rotation angles and all the predetermined periods, the minimum Hamming distance is taken as the first maximum similarity.

Wherein, a frequency domain image may be obtained using a discrete cosine transform, i.e., a first frequency domain image is obtained by performing a discrete cosine transform on a translated third reference image, a second frequency domain image is obtained by performing a discrete cosine transform on a visualization image of gene expression; and then a low-frequency frequency domain image is intercepted from the first frequency domain image, and a pixel mean value of the low-frequency frequency domain image is calculated by taking the low-frequency frequency domain image as a reference; in the low-frequency frequency domain image, the pixel value for a pixel point with a pixel value higher than the pixel mean value is set to 1, the pixel value for a pixel point with a pixel value lower than the pixel mean value is set to 0, and the pixel value for a pixel point with a pixel value equal to the pixel mean value is set to 0 or 1; and then a hash fingerprint is obtained based on the low-frequency frequency domain image with reset value. The hash fingerprint of the second frequency domain image is obtained according to the same execution steps. Then the hash fingerprint of the first frequency domain image and the hash fingerprint of the second frequency domain image are compared to determine the bits number of different values between the two hash fingerprints, which is served as the Hamming distance between the first frequency domain image and the second frequency domain image. It should be noted that the method of calculating similarity in the above embodiment is only an exemplary implementation, and the method of calculating image similarity is not limited by the above implementation, and other feasible methods are also within the scope of protection of the present disclosure.

In the above embodiment of the present disclosure, the fourth reference image may first be preliminarily translated and positioned in accordance with a centroid offset from the visualization image of gene expression. In another possible implementation, in determining the third reference image, the fourth reference image is not preliminarily positioned, but is translated in accordance with each of the periods within the predetermined periods for any predetermined rotation angle, to obtain the seventh reference image corresponding to each of the periods. For example, a third centroid coordinate of the fourth reference image may be calculated first, and centering on the third centroid coordinate, the fourth reference image is translated according to each period within the predetermined periods respectively to obtain the seventh reference image. A second similarity between the seventh reference image and the visualization image of gene expression is then calculated, thereby obtaining the second similarity corresponding to each period within the predetermined periods. The above steps are then repeated according to other predetermined rotation angles to obtain the second similarity corresponding to each predetermined rotation angle as well as each period, and a second maximum similarity is determined based on the above plurality of second similarities. The seventh reference image corresponding to the second maximum similarity is taken as the third reference image, and then the object offset is determined based on this third reference image and the visualization image of gene expression.

Specifically, the plurality of third offsets may first be determined based on the visualization image of gene expression and the third reference image, and the object offset may be determined based on the plurality of third offsets. Wherein, the principle of calculating the third offset is the same as the principle of calculating the fourth offset in the above embodiment. In other words, the plurality of groups of track crosses sets are obtained, wherein each group of track crosses sets includes a third track cross and a fourth track cross, wherein the third track cross is located in the third reference image and the fourth track cross is located in the visualization image of gene expression, and wherein the third track cross and the fourth track cross have the same index identifier. For either group of track crosses sets, the third offset is determined based on the coordinates of the third track cross and the coordinates of the fourth track cross. Wherein, the specific implementation of determining the third offset based on the coordinates of the third track cross and the coordinates of the fourth track cross may refer to the specific implementation of determining the fourth offset based on the coordinates of the first track cross and the coordinates of the second track cross, which will not be repeated herein.

Similarly, after determining the third offsets corresponding to the plurality of groups of track crosses sets, the third offsets that are out of a predetermined range may be first excluded, and then a median offset of the plurality of third offsets that are within the predetermined range may be selected as the object offset, or an average offset of the plurality of third offsets may be calculated to serve as the object offset, to maximize the accuracy of offset determination.

Step 1024, translating the third reference image by the object offset to obtain the registered image.

After determining the object offset based on the method provided in the above embodiment, the predetermined rotation angle corresponding to the object offset is determined, and the third reference image is translated based on the predetermined rotation angle and the object offset.

It should be noted that during the image translation in the intermediate process, all processing are performed on the temporary image of the replicated stained image, i.e., the stained image is not translated for registration. When the calculation is completed to obtain the final object offset, and then the stained image is translated at one time to be registered with the visualization image of gene expression.

In the image processing method provided in embodiments of the present disclosure, the stained image may be registered with the visualization image of gene expression based on image coordinates with pixel-level accuracy, which improves the accuracy of the image processing and facilitates subsequent research and analysis using the registered image.

When the third reference image and the visualization image of gene expression have a plurality of periods, the offsets corresponding to the predetermined periods are obtained from the calculated offsets by theoretical reasoning of the period width, there may be errors in the actual images. In order to correct the error in the theoretical reasoning and further improve the accuracy of the image processing, in a preferred embodiment, after translating the third reference image by the object offset, the translated third reference image as well as the visualization image of gene expression may be reused to determine the corrected offset as the final offset. The third reference image is translated again based on the determined final offset, and the translated third reference image is registered with the visualization image of gene expression to improve the accuracy of the registration. For example, based on the translated third reference image and the visualization image of gene expression, a plurality of groups of track crosses sets are obtained, wherein each group of track crosses sets includes a fifth track cross and a sixth track cross, the fifth track cross is located in the third reference image, the sixth track cross is located in the visualization image of gene expression, and the fifth track cross and the sixth track cross have the same index identifier. For any one of the plurality of groups of track crosses sets, a fifth offset is determined based on the coordinates of the fifth track cross and the coordinates of the sixth track cross. After determining the fifth offset corresponding to each of the plurality of groups of track crosses sets, a final offset is determined based on the plurality of fifth offsets. Among other things, the implementation of determining the fifth offset and determining the final offset based on the plurality of fifth offsets may refer to the above embodiments and will not be repeated herein.

As a feasible means of embodiments of the present disclosure, after the step of scaling the stained image to obtain a third reference image, the method further includes: determining a similarity between the third reference image and the visualization image of gene expression; and taking a position with a maximum similarity as a result of a coarse registration. This similarity is used for the coarse registration and the method shown in FIG. 11 is used for the fine registration.

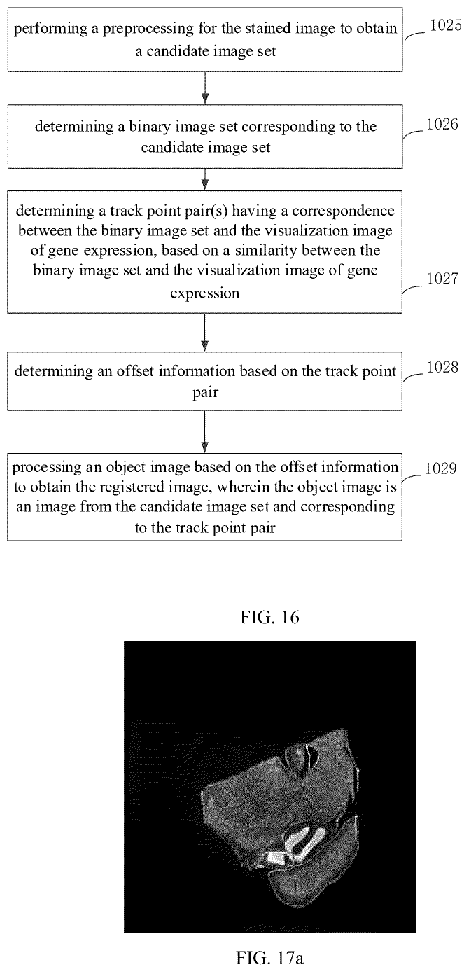

In some embodiments, as shown in FIG. 16, a schematic flow diagram of a method for performing image registration for the stained image and a visualization image of gene expression according to an embodiment of the present disclosure, the method includes steps as follows.

Step 1025, performing a preprocessing for the stained image to obtain a candidate image set.

Since the stained image is an image actually filmed by a microscope and is affected by external and other factors, there are certain differences from the visualization image of gene expression. For example, the stained image is not in a horizontal position, so in order to facilitate subsequent image registration, the stained image may be preprocessed first, before performing registration for the stained image and the visualization image of gene expression.

Specifically, the first track line and the second track line of the stained image are obtained, and the first track line and the second track line may be randomly determined track lines. The stained image is then rotated based on the angle between the first track line and the horizontal direction, in other words, the stained image is rotated to a horizontal position. And the first spacing between the first track line and the second track line is determined. The third track line and the fourth track line in the visualization image of gene expression are obtained, wherein the index number of the third track line is the same as the index number of the first track line, and the index number of the fourth track line is the same as the index number of the second track line. And the second spacing between the third track line and the fourth track line is determined. The rotated stained image is adjusted based on the ratio of the first spacing and the second spacing to obtain a candidate image. In other words, there may be a scaling ratio difference between the spacing of the track lines of the stained image and the visualization image of gene expression, therefore the stained image may be scaled to the same ratio as the visualization image of gene expression in order to achieve accurate image registration. Wherein, when determining the first spacing between two track lines based on the first track line and the second track line, two track crosses having the same horizontal coordinates in the first track line and the second track line may be acquired, and the difference between the vertical coordinates of the two track lines is determined as the first spacing. Alternatively, two track crosses having the same vertical coordinates in the first track line and the second track line may be acquired, and the difference between the horizontal coordinates of the two track lines is determined as the first spacing.

After obtaining the candidate image, the candidate image, being in a horizontal position at this time, may differ from the visualization image of gene expression by angle multiples of 90°, therefore a plurality of rotation angles may be determined, and the candidate image may be rotated based on each of the rotation angles, thereby obtaining the candidate image set consisting of the rotated images corresponding to each of the rotation angles. For example, the plurality of rotation angles may be 90°, 180°, and 270°. After rotation, any of the images in this candidate image set still has the same index number as the stained image.

Step 1026, determining a binary image set corresponding to the candidate image set.

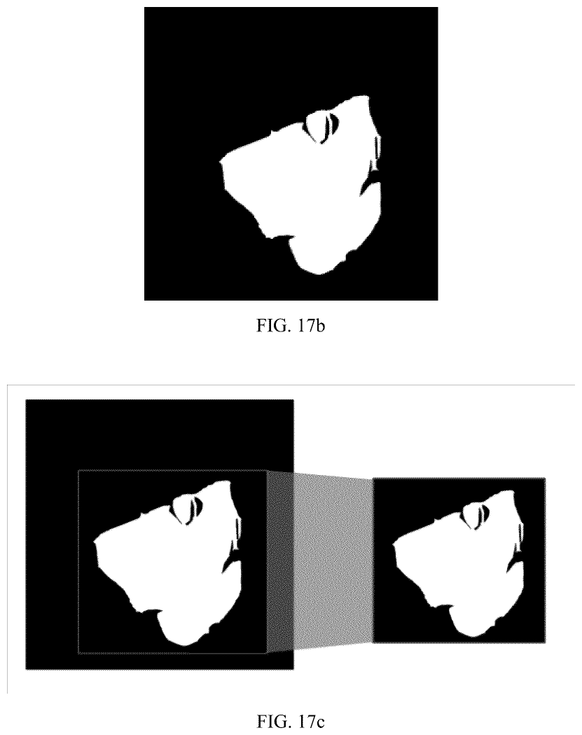

The binary image is obtained by an image binaryzation, including setting the grayscale value of each pixel point on the image to 0 or 255, so that the entire image presents a distinct black and white effect, which is convenient for subsequent image registration. Optionally, the binary image set may be determined in the following approach: For any fourth image in the candidate images set, segmentation is performed based on the tissue boundary of the fourth image, and the tissue segmentation image is obtained, wherein, the tissue segmentation may be performed based on a tissue segmentation algorithm, or manually tissue boundary circling using a tissue segmentation tool. The tissue segmentation image is then binarized to obtain an initial binary image. For any one of the plurality of edges of the initial binary image, edge points within the tissue boundary that is closest to the boundary is determined; and an edge track line that is closest to the edge point is determined among the track lines that are parallel to the edges, ensuring that the edge track line does not pass through the tissue boundary; so that determining a target binary image based on the edge track line corresponding to each edge. The binary image set consist of the target binary images corresponding to the plurality of fourth images in the candidate images sets. In other words, the target binary image is a minimum binary image including the entire biological tissue, and the edges of the target binary image consist of the track lines. Referring to what is shown in FIG. 17a, a schematic diagram of a fourth image according to an embodiment of the present disclosure, the segmentation processing is performed for the tissue boundary of the fourth image to obtain the tissue segmentation image, and then the tissue segmentation image is subjected to a binaryzation processing. The initial binary image obtained is shown as FIG. 17b. The minimum binary image including the entire tissues, which is the target binary image as shown in FIG. 17c, is then determined based on the initial binary image.

Step 1027, determining a track cross pair(s) having a correspondence between the binary image set and the visualization image of gene expression, based on a similarity between the binary image set and the visualization image of gene expression.

In practice, the visualization image of gene expression may have a plurality of periods, each period of the visualization image of gene expression being the same, that is, the index number of the visualization image of gene expression may have a plurality of periods. Based on this, when determining the track cross pairs, for any binary image in the binary images set, a plurality of regions corresponding to the binary image are determined in the visualization image of gene expression based on the coordinates corresponding to the vertex of the binary image and the coordinates corresponding to the track crosses of the visualization image of gene expression. Wherein the plurality of regions determined are one-to-one corresponded with the plurality of periods. A vertex of the binary image denotes a track cross in the binary image, and the coordinates corresponding to the vertex may be determined by index numbers corresponding to two track lines that intersect to form the vertex. For any one of the plurality of regions described above, a similarity index between the binary image and the region is determined, so that a plurality of similarity indexes between the plurality of binary images and the plurality of regions two by two can be determined. Based on the plurality of similarity indexes, track crosses pairs having a correspondence between the binary images set and the visualization image of gene expression are determined. Wherein the similarity index may indicate a degree of similarity between the image corresponding to the binary image and the image corresponding to the region, and the larger the similarity index, the higher the degree of similarity.

In determining the similarity index between the binary image and the region, this can be implemented by following approach: determining a binary image matrix corresponding to the binary image, and since the pixel points of the binary image can only have a grayscale value of either 0 or 255, optionally, the pixel points with a grayscale value of 0 may be set to an element of 0 in the binary image matrix, and the pixel points with a grayscale value of 255 may be set to an element of 1 in the binary image matrix. In other words, the binary image is a matrix consisting of 0 and 1. When the visualization image of gene expression is a visualization image of gene expression, a gene expression matrix corresponding to the visualization image of gene expression may be determined, and each pixel point of the visualization image of gene expression corresponds to a gene expression level in the gene expression matrix. The similarity indexes are then determined using the binary image matrix x and the gene expression matrix corresponding to the region. For example, a dot products may be performed between the binary image matrix an the gene expression matrix, i.e., the binary image matrix is multiplied by elements at corresponding positions in the gene expression matrix, and then all elements in the matrix obtained by the dot products are summed to obtain the similarity indexes. Thereby, track crosses pairs having correspondence may be determined based on the similarity indexes between the binary images set and a plurality of regions of the visualization image of gene expression.

According to the above embodiment, the stained image may be obtained by performing processing to the first original image using the track line positioning algorithm, or by scaling the first original image according to the predetermined ratio and then performing the processing using the track line positioning algorithm. In the following, for each of the above two approaches of obtaining the stained image and the visualization image of gene expression, the approach of determining the track crosses pairs based on a plurality of similarity indexes will be described.

When performing processing to the first original image and the second original image using the track line positioning algorithm to obtain the stained image and the visualization image of gene expression, the plurality of similarity indexes between the plurality of binary images in the binary images set and the plurality of regions are obtained, then a maximum value among the plurality of similarity indexes may be obtained, the binary image corresponding to the maximum value is determined as a first object image and the region corresponding to the maximum value is determined as the first target region. Then a first vertex in the first object image and a first index point in the first target region having the same coordinates as the first vertex may be obtained, and the first vertex and the first index point are determined as the track cross pair having a correspondence. The first vertex may be any vertex in the first object image.

When scaling the first original image and the second original image according to the predetermined ratio, performing processing for the scaled first original image and the scaled second original image using the track line positioning algorithm, to obtain the stained image and the visualization image of gene expression, which includes determining a ranking result of the plurality of similarity indexes after obtaining the plurality of similarity indexes between the plurality of binary images in the binary images sets and the plurality of regions; obtaining a predetermined number of similarity indexes based on the ranking result; and determining the plurality of binary images and the plurality of regions, which are corresponding to the predetermined number of similarity indexes. For example, the top predetermined number of similarity indexes with the maximum value may be selected. Among other things, the embodiments of the present disclosure do not limit the specific numerical value of the predetermined number, which may be determined in combination with the actual application scenario. Since there may be errors during the registration using the scaled image, in order to further improve the accuracy of the image processing, the images before the binary image scaling corresponding to the predetermined number of similarity indexes may be obtained. In other words, after determining the plurality of binary images corresponding to the predetermined number of similarity indexes, the plurality of binary images are scaled up according to the reciprocal of the predetermined scaling ratio, and a plurality of second object images, i.e., images before scaling, are determined. The plurality of candidate similarity indexes are then determined based on the binary image matrix corresponding to the plurality of second object images and the gene expression matrix corresponding to the plurality of regions. A maximum value in the plurality of candidate similarity indexes is obtained, and the binary image corresponding to the maximum value is determined as a third object image, and the region corresponding to the maximum value is determined as a third target region. A second vertex in the third object image and a second index point in the third target region having the same coordinates as the second vertex are obtained, and the second vertex and the second index point are determined as the track cross pair having a correspondence. The second vertex may be any vertex in the third object image.

Step 1028, determining an offset information based on said track cross pair.

Optionally, the coordinates corresponding to the two track crosses of the rule point pair may be obtained, and the difference of the horizontal coordinates and the difference of the vertical coordinates may be determined to be the offset information, i.e., the offset.

Step 1029, processing an object image based on the offset information to obtain the registered image, wherein the object image is an image from the candidate image set and corresponding to the track cross pair.

After determining the offset information, the image registration may be performed by translating the object image, i.e. the image corresponding to the track crosses pairs in the candidate images set, based on the offset information.

It should be noted that during the image processing and translation in the intermediate process, both are performed on the temporary image of the replicated stained image, i.e., the stained image is not translated for registration. When the calculation is completed to obtain the final object offset, and then the stained image is translated according to the rotation direction of the object image as well as the offset information, to be registered with the visualization image of gene expression.

With the image processing method provided by the present disclosure, image processing at pixel-level accuracy may be realized without the need to manual image registration, thereby improving the accuracy of image processing.

Before performing cell segmentation for said registered image to obtain a cell mask image, said method further includes: step 105, performing tissue segmentation for said registered image to obtain a tissue mask image.

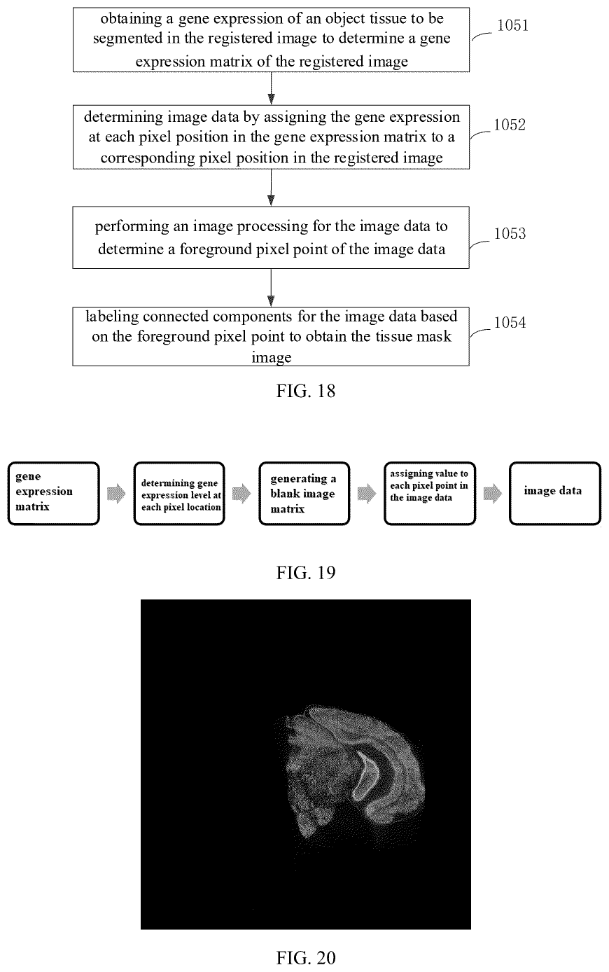

In some embodiments, as shown in FIG. 18, a schematic flow diagram of a method of performing tissue segmentation for a registered image according to an embodiment of the present disclosure. The method includes steps as follows.

Step 1051, obtaining a gene expression of an object tissue to be segmented in the registered image to determine a gene expression matrix of the registered image, wherein the gene expression in the gene expression matrix is arranged according to a pixel position.

In an embodiment, the object tissue may include, but is not limited to, any one or more of sample tissue, biological tissue, muscle tissue, skeletal tissue, and cell tissue. The gene expression matrix characterizes the expression of genes at different locations (corresponding to row and column positions of the gene expression matrix). Each piece of gene expression data includes a gene identifier, gene coordinates, and gene expression level, characterizing the expression of a gene at a location. In the case of cell tissues, for example, the DNA of the cell tissue may be transcribed to determine the gene expression level of the cell tissue. In addition, the gene expression level is typically in utf-8 format.

In this embodiment, a gene expression matrix is obtained by obtaining the gene expression level at each pixel location in the sample image and arranging the gene expression level according to the row coordinates and column coordinates of the pixel locations in order to obtain the gene expression matrix, which is consistent with the size of the sample image. When the gene expression level at each pixel location is 0, the gene of the object tissue is characterized as not being present at the pixel location. The gene expression level at each pixel location in the gene expression matrix may characterize the gene expression level of one or more genes. If the gene expression matrix is not consistent with the size of the sample image, the both images or one of the images may be scaled.

Step 1052, determining image data by assigning the gene expression at each pixel position in the gene expression matrix to a corresponding pixel position in the registered image.

Determine an image matrix of the same size as said gene expression matrix.

Assigning the gene expression level at each pixel position in the gene expression matrix to the corresponding pixel position in the image matrix, and performing a normalization at all pixel positions to obtain the image data of the sample image.

Referring to FIG. 19, the gene expression at each pixel position in the gene expression matrix is stored in a dictionary with the row coordinates and column coordinates of each pixel position as the key and the gene expression as the value. An all-0 image matrix of the same size as the gene expression matrix is created, and the same positions in the image matrix are assigned according to the positions of the row coordinates and column coordinates of the gene expression quantities stored in the dictionary. In other words, the gene expression level at the same pixel positions in the image matrix and the gene expression matrix are same. The image matrix after assignment is normalized so that the pixel value at each pixel position is within the interval of [0,255], to obtain more accurate image data relative to the sample image and reduce the impact on the subsequent tissue segmentation image. The image data is shown in FIG. 20, and the format of the image data is preferably in TIFF format, or JPG format, etc., and is not limited to a specific form of expression, but may be selected according to the actual situation.

In one embodiment, the gene expression matrix is generated by obtaining the gene expression level at each pixel location, and the gene expression matrix is converted to image data, which reduce the repeatedly filming and correcting of the tissue image with the original microscope, improve the segmentation efficiency, and make the image data more accurate for the display of the object tissue as compared to the initial sample image, and the segmentation effect for the object tissue is better.

Step 1053, performing an image processing for the image data to determine a foreground pixel point of the image data.

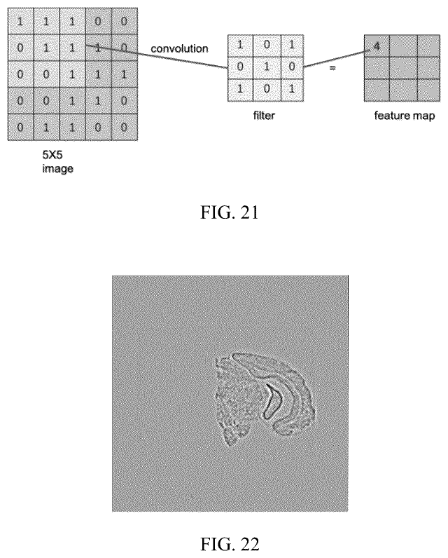

In one embodiment, before step S1053, the tissue segmentation method further includes: referring to FIG. 21, convolving the image data to extract low-frequency features of the image data. The convolution method preferably adopts 2d convolution, and convolution methods such as cavity convolution, depthwise separable convolution, and the like may also be adopted to enhance the computational voxel and expand the field of perception, and the image data after convolution is shown in FIG. 22.

The image processing is performed to the image data after convolution to determine the foreground pixel points of the image data.

Step 1054, labeling connected components for the image data based on the foreground pixel point to obtain the tissue mask image.

In one embodiment, the connected component refers to an image region including foreground pixel points having the same pixel value and adjacent positions in the image data, i.e., a segmented region of the object tissue.

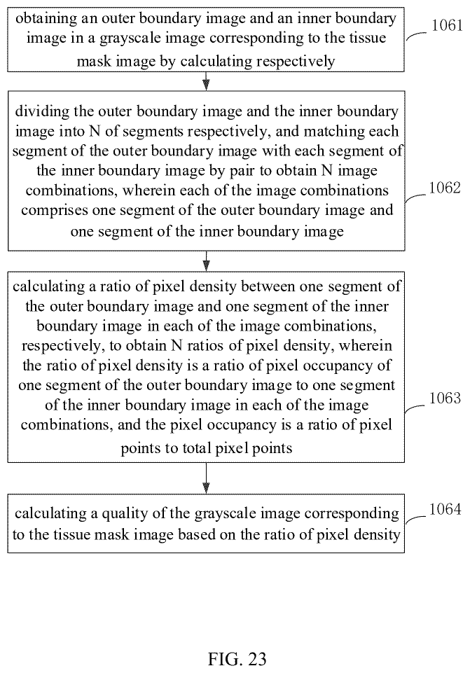

In some embodiments, after obtaining the tissue mask image with tissue segmentation, as shown in FIG. 23, a schematic flow diagram of a method of calculating a quality of a grayscale image corresponding to a tissue mask image according to an embodiment of the present disclosure, the method includes steps as follows.

Step 1061, obtaining an outer boundary image and an inner boundary image in a grayscale image corresponding to the tissue mask image by calculating respectively.

The outer boundary image and the inner boundary image of said grayscale image of the target regional tissue may be obtained respectively based on matrix operations. In this embodiment, said outer boundary image and said inner boundary image are banded annular contour, and said inner boundary image may be surround by said outer boundary image.

Step 1062, dividing the outer boundary image and the inner boundary image into N of segments respectively, and matching each segment of the outer boundary image with each segment of the inner boundary image by pair to obtain N image combinations, wherein each of the image combinations includes one segment of the outer boundary image and one segment of the inner boundary image.

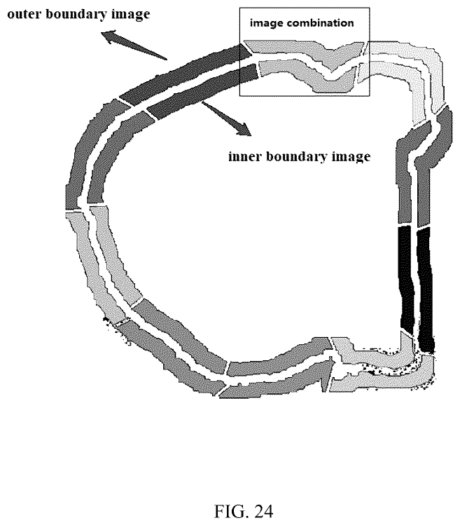

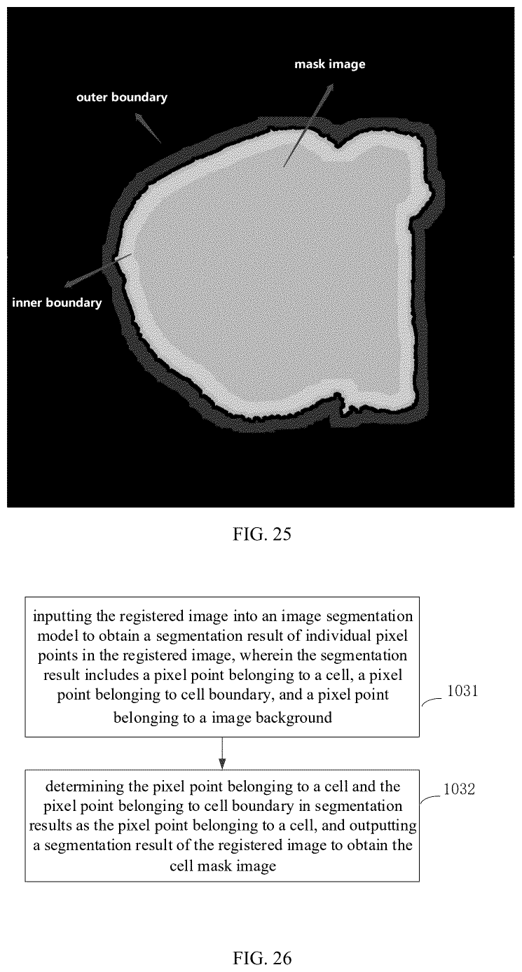

To show the relationship between the outer boundary image and the inner boundary image more intuitively, FIG. 24 shows a structural schematic diagram of a relationship between an outer boundary image and an inner boundary image according to an embodiment of the present disclosure, and FIG. 25 shows a structural schematic diagram of another relationship between an outer boundary image and an inner boundary image according to an embodiment of the present disclosure. As shown in FIG. 24, said dividing the outer boundary image and the inner boundary image into N of segments respectively includes dividing the outer boundary image evenly into N of segments; dividing the inner boundary image evenly into N of segments; matching one segment of said N of segments of outer boundary image and one segment of said N of segments of inner boundary image by pair to obtain the image combinations, wherein N image combinations may be obtained after the pairing.

Step 1063, calculating a ratio of pixel density between one segment of the outer boundary image and one segment of the inner boundary image in each of the image combinations, respectively, to obtain N ratios of pixel density, wherein the ratio of pixel density is a ratio of pixel occupancy of one segment of the outer boundary image to one segment of the inner boundary image in each of the image combinations, and the pixel occupancy is a ratio of pixel points to total pixel points.

Before calculating the ratio of pixel density, it may be necessary to calculate the pixel occupancies of each segment of the outer boundary image and each segment of the inner boundary image. Said calculating the pixel occupancy of one segment of the outer boundary image includes dividing the number of pixel points within this segment of the outer boundary image by the total number of pixel points within this segment of the outer boundary image, and said pixel points are pixel points with a grayscale value of greater than 127 in the present embodiment. Calculating the pixel occupancy of one segment of segment of inner boundary image in the same way as calculating the pixel occupancy of one segment of segment of the outer boundary image, i.e. dividing the number of pixel points within this segment of the inner boundary image by the total number of pixel points within this segment of the inner boundary image, and so on to calculate the pixel occupancies of all segments of the outer boundary image and all segments of the inner boundary image finally.

After the calculation of the above pixel occupancy, it may be also necessary to calculate the ratio of pixel occupancy of one segment of the outer boundary image to one segment of the inner boundary image in each of the image combinations, which denotes as the ratio of pixel density. Said one segment of the outer boundary image and said one segment of the inner boundary image are included in one image combination, i.e., both images are paired with each other. Finally, N ratios of pixel density are obtained by calculating the ratio of pixel occupancy of one segment of the outer boundary image to one segment of the inner boundary image in all of the image combinations.

Step 1064, calculating a quality of the grayscale image corresponding to the tissue mask image based on the ratio of pixel density.

Said calculating a quality of the grayscale image corresponding to the tissue mask image based on the ratio of pixel density includes, but is not limited to, the following implementations. For example, plugging the average of the ratio of pixel density into predetermined function to calculate a value describing the quality of the grayscale image corresponding to the target regional tissue, said predetermined function may be a linear function or a non-linear function.

In order to facilitate the understanding of the above refinement, the present embodiment provides an exemplary illustration in view if a formula, e.g., the average value of the ratio of effect pixel density is x, and when said x is greater than 0 and less than 200, a numerical value describing the quality of the gray-scale image of said target regional tissue may be obtained by plugging the X into formula (1), which is shown below:

$$F ( x ) = 100 * x \hat{} 0.7 ( 0 < x < 2 \; 00 ) \qquad Formula ( 1 )$$

$$F ( x ) = 0 ( x < = 0 ) \qquad Formula ( 2 )$$

$$F ( x ) = 100 ( x > = 200 ) \qquad Formula ( 3 )$$

The above formulas are illustrative only and may be replaced by any function, and embodiments of the present disclosure do not limit the formulas.

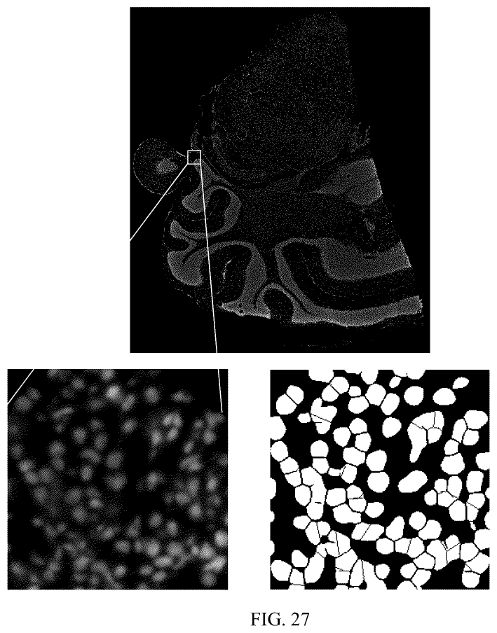

In some embodiments, as shown in FIG. 26, a schematic flow diagram of a method of performing a cell segmentation for a registered image according to an embodiment of the present disclosure, the method includes steps as follows.

Step 1031, inputting the registered image into an image segmentation model to obtain a segmentation result of individual pixel points in the registered image, wherein the segmentation result includes a pixel point belonging to a cell, a pixel point belonging to cell boundary, and a pixel point belonging to a image background.

By inputting the registered image into the above-described image segmentation model, a probability value showing that each pixel point in the registered image belongs to a pixel point belonging to a cell, a pixel point belonging to cell boundary, and a pixel point belonging to a image background may be obtained; and an initial segmentation result of each pixel point in the registered image is determined according to whether the probability value is greater than a threshold value, in other words, each pixel point in the registered image belongs to a pixel point belonging to a cell, a pixel point belonging to cell boundary, and a pixel point belonging to a image background may be obtained are determined.

In practice, the registered image may be expanded to a three-channel (e.g., RGB three-channel) image, and be input into the image segmentation model, so that obtaining the initial segmentation results of each pixel point in images of each channel, i.e., each pixel point belongs to a target object, a target object outline or an image background in images of each channel; and then the initial segmentation results of each pixel point in images of each channel are summarized to determine the initial segmentation results of each pixel point in the registered image.

Step 1032, determining the pixel point belonging to a cell and the pixel point belonging to cell boundary in segmentation results as the pixel point belonging to a cell, and outputting a segmentation result of the registered image to obtain the cell mask image.

Pixel points belonging to a cell or cell boundary are fused to perform the segmentation of the cell, i.e., pixel points of a cell object or cell boundary are identified as pixel points belonging to the cell, then image segmentation is performed, and the segmentation results of the registered image are output.

In embodiments of the present disclosure, the watershed algorithm may further be utilized to filter target objects with an area smaller than a threshold in the segmentation result of the registered image, and/or, to repair incomplete target objects.

By filtering target objects with an area smaller than a threshold value and/or repairing incomplete target objects, the target object is corrected and the post-processing of segmentation results of the registered image is performed, thereby improving the accuracy of image segmentation. For example, when the target object is a cell, the cell segmentation becomes more accurate by filtering very small cells and fixing incomplete cells through algorithms, such as cell filtering and correction algorithms.

In the image segmentation model in embodiments of the present disclosure, a pyramid squeeze attention block is employed to improve the feature extraction capability of the encoder module, so that the image segmentation model pays more attention to the target object with significant features, thereby improving the segmentation accuracy of the image segmentation model.

The embodiments of the present disclosure are applied to cell segmentation in spatial-temporal omics, see FIG. 27, a diagram of a segmentation performance of an image segmentation model on a mouse brain image according to an embodiment of the present disclosure. A part of the mouse brain image in the upper figure was intercepted as a demonstration, and as can be seen, there is a presence of cell adhesion in the mouse brain image and the boundaries of some cells are severely indistinct as shown in the lower left figure, however independent cells can be segmented from the adhesive cells using the image segmentation model proposed by the embodiments of the present disclosure, providing a more reliable boundary, as shown in the lower right figure.

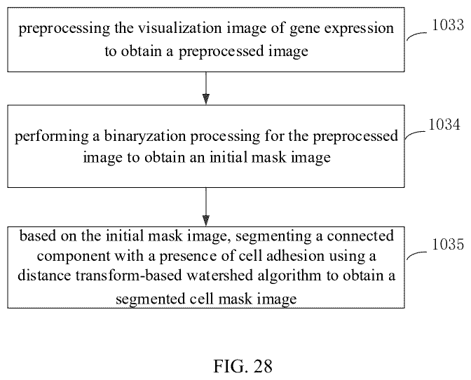

In some embodiments, as shown in FIG. 28, a schematic flow diagram of another method for performing a cell segmentation for a registered image according to an embodiment of the present disclosure, the method includes steps as follows.

Step 1033, p preprocessing the visualization image of gene expression to obtain a preprocessed image.

Since the visualization image of gene expression is a scatter plot, which is not convenient for cell segmentation, preprocessing is required for the visualization image of gene expression of the cell to obtain a preprocessed image with enhanced effect of boundary.

Step 1034, performing a binaryzation processing for the preprocessed image to obtain an initial mask image.

The present embodiment may use the Otsu method to perform a binaryzation for the preprocessed image to obtain an initial mask image.

Step 1035, based on the initial mask image, segmenting a connected component with a presence of cell adhesion using a distance transform-based watershed algorithm to obtain a segmented cell mask image.

Watershed Algorithm is based on the composition of the watershed to consider the segmentation of the image. Watershed Algorithm is based on the composition of the watershed to consider the segmentation of the image.

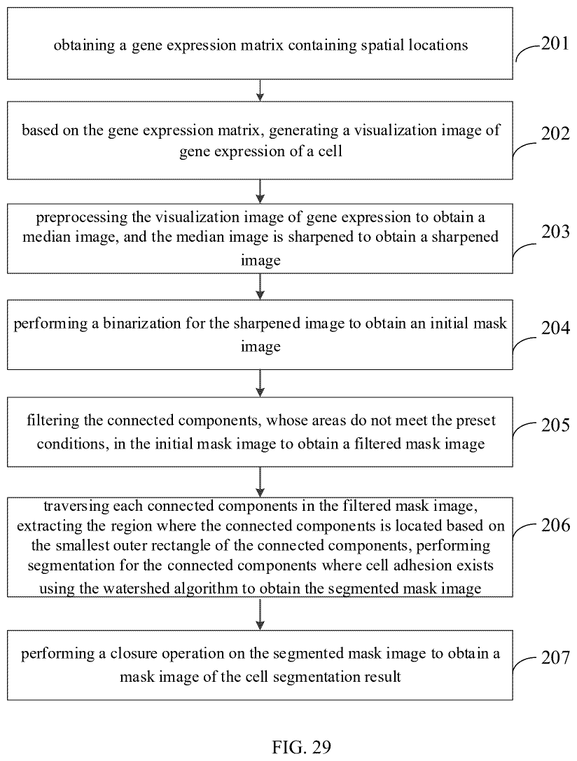

Further, as a refinement and extension of the above embodiment, in order to completely illustrate the specific implementation process of the method of the present embodiment, the present embodiment provides a specific method as shown in FIG. 29, including steps as follows.

Step 201, obtaining a gene expression matrix containing spatial locations.

The gene expression matrix containing spatial locations is obtained from the gene expression data of the cell. The gene expression data may include a gene identifier for a plurality of genes, a coordinate position and a total gene expression at the corresponding coordinate position.

Step 202, based on the gene expression matrix, generating a visualization image of gene expression of a cell.

As an optional way, step 202 may specifically include: first obtaining the coordinate positions of the expressed genes in the gene expression matrix and the total gene expression level at the corresponding coordinate positions; then generating the visualization image of gene expression based on the coordinate positions of the expressed genes and the total gene expression level at the corresponding coordinate positions, wherein the visualization image of gene expression is a grayscale map, and the grayscale values of the pixel points in the visualization image of gene expression are the total gene expression level at the coordinate position corresponding to the pixel point. In this optional method, the visualization image of gene expression of the obtained cell may be accurately generated.

For example, in use for a specific cell segmentation, a gene expression matrix containing spatial locations is input, and an expression image is generated based on the coordinate positions of the expressed genes and the total gene expression at the corresponding locations. The expression image is specifically in the form of a grayscale image, and the grayscale values of the coordinate points are the total gene expression level at the respective coordinates.

As another alternative, step 202 may specifically further include: mapping a visualization image of gene expression of the cell based on the gene expression matrix and the segmented mask image, wherein the spatial location in the gene expression matrix corresponds to the spatial location in the segmented mask image. For example, a cell-based expression image is mapped based on the gene expression matrix and the segmented mask image, wherein the spatial locations in the gene expression matrix may correspond to the spatial locations in the segmented mask image. In this optional manner, a visualization image of gene expression of the obtained cell can be accurately generated.

The generated visualization image of gene expression is a scatterplot, which is not convenient to be segmented, therefore processing is required, by performing the process shown in step 203.

Step 203, preprocessing the visualization image of gene expression to obtain a median image, and the median image is sharpened to obtain a sharpened image.

The generated visualization image of gene expression is a scatterplot, which is not convenient to be segmented, therefore preprocessing is required. In the present embodiment, the visualization image of gene expression of the cell is first processed into a median image. The median image may then be sharpened using the laplacian operator to enhance the effect of boundary of the median image, and thus a sharpened image is obtained.

Optionally, the preprocessing the visualization image of gene expression to obtain a median image specifically includes:
performing a convolution for the visualization image of gene expression using a convolution kernel of a predetermined size (e.g., 13*13) firstly; enabling the scatter points in the visualization image of gene expression to be adhered, thereby obtaining a first convolution image;
then detecting a local maximum point of the first convolution image according to the two-dimensional gray peak value of the image; obtaining a p-th percentile number of the local maximum point, wherein p is a predetermined value, such as the p-th percentile may be a 98% quantile value, or a 99% quantile value, etc., if the p-th percentile is within a predetermined range, the median filtering on the first convolution image will be performed using the first median filter to obtain the median image, wherein a filter size of the first median filter is determined according to a predetermined size.

Further optionally, after obtaining the p-th percentile of the local maximum point, the method of the present embodiment may further include: if the p-th percentile is out of a predetermined range, determining a new size of the convolution kernel based on the p-th percentile and the predetermined size; performing a convolution operation on the visualization image of gene expression using the convolution kernel of the new size, to enable the scatter points in the visualization image of gene expression to be adhered together, thereby obtaining a second convolution image; performing median filtering on the second convolution image using a second median filter to obtain the median image, wherein a filter size of the second median filter is obtained by determining the new size.

In some embodiments, determining the new size of the convolution kernel based on the p-th percentile and the predetermined size may specifically include: calculating the new size of the convolution kernel according to the formula K=N*(N/R), wherein K*K denotes the new size of the convolution kernel, N*N denotes the predetermined size, and R denotes the p-th percentile.

For example, performing convolution for the original image of the visualization image of gene expression using the convolution kernel of size 13*13 (empirical value) first to obtain the first convolution image; and then according to the image of the two-dimensional gray peak detection of the first convolution image of the local maximum points, taking out the 99% of all the local maximum points obtained from the quantile value R, if the gap between the value of R and a predetermined empirical threshold is too large (too high or too low, both affect the subsequent processing), then the 13*13 convolution kernel will be considered as being not applicable to the original image, K=13*(13/R) will be calculated and the original 13*13 convolution kernel will be changed to K*K for re-processing the original image, thereby obtaining the second convolution image; if the R value is within the permissible range, then the first convolution image will be used forward. The convolution image is in the form of a cascaded squares with different grayscales, which span enormously, and there is case that the cavity in center of the aggregated points cluster present in some of the expression image. To solve this problem, the second convolution image is subjected to a median filter using the filter size of 13*2.7=35 (2.7 may be an empirical value, with the decimals rounded downward) (if K needs to be computed, then the kernel size is K*2.7 in this case), to obtain the median image.

In order to fill the cavity in the of points cluster, the median filter used above is larger, and the grayscale boundaries of the median image may be more indistinct. The median image is sharpened using the laplacian operator to enhance the grayscale boundaries, and a sharpened map is obtained to complete the preprocessing, and the following process of cell segmentation is performed, and specifically, the process shown in steps 204 to 207 may be performed.

Step 204, performing a binaryzation for the sharpened image to obtain an initial mask image.

The sharpened image obtained in the previous step is binarized using the Otsu method to obtain the initial mask image.

Step 205, filtering the connected components, whose areas do not meet the preset conditions, in the initial mask image to obtain a filtered mask image.

Optionally, step 205 may specifically include filtering the connected component that has an area larger than a first preset threshold, or the connected component that has an area smaller than a second preset threshold in the initial mask image, to obtain the filtered mask image, wherein the first preset threshold is larger than the second preset threshold. For example, connected component with an area that is too small or too large in the initial mask are filtered using an empirical threshold to obtain the filtered mask image.

Step 206, traversing each connected components in the filtered mask image, extracting the region where the connected components is located based on the smallest outer rectangle of the connected components, performing segmentation for the connected components where cell adhesion exists using the watershed algorithm to obtain the segmented mask image.

Optionally, step 206 may specifically include:

setting the grayscale value of each pixel point within the connected components in the filtered mask image as a first value, and setting the grayscale value of the pixel point outside each connected components as a second value; for each target pixel point within the connected components with a grayscale value of the first value, remapping the grayscale value of the target pixel point as a distance between the target pixel point and the nearest pixel point with the second value; performing a binarization for the distance map of the connected components to obtain a preset number of pixel points, that are furthest from the pixel point with the second value, in the connected components; taking the preset number of pixel points as the watering point of the watershed algorithm, and performing a watershed segmentation for the original mask of the connected components in the filtered mask image using the watershed function to obtain the segmented target connected components; and covering the target connected components to the filtered mask image to obtain the segmented mask image.

For example, said traversing each connected components in the filtered mask image, extracting the region where the connected components is located based on the smallest outer rectangle of the connected components, performing segmentation for the connected components where cell adhesion potentially exists using the distance transform-based watershed algorithm specifically includes steps as follows.

Step a, for each extracted connected domain region, the grayscale value of the points within the target connected domain is set to 1, and the grayscale value of the points outside the connected domain (including the background and the non-target connected domain) is set to 0, and a distance transform is performed on each point with a value of 1, so that the grayscale value is remapped as the distance between said point and the nearest point with the value of 0 (i.e. the distance of the adjacent points is 1), to obtain the distance map of the connected domain.

Step b, the distance map is binarized with an empirical threshold to obtain certain number of points in the connected domain that are furthest away from the point with a distance value of 0 (the respective number is variable due to the different situation in each connected domain).

Step c, the certain number of points obtained in the previous step are used as the watering points of the watershed algorithm, watershed segmentation is performed on the original image of the connected domain using the watershed function in opencv to obtain the segmented target connected domain, and the filtered mask image is covered by this result.

Step d, for each connected domain traversed, the above steps are performed to obtain the segmented mask image.

Step 207, performing a closure operation on the segmented mask image to obtain a mask image of the cell segmentation result.

To make the cell boundaries more regular, the final mask image is obtained by performing a closure operation on the segmented mask. Finally, the final mask image can be output and saved for subsequent biological analysis at the cellular level in combination with the original expression matrix.

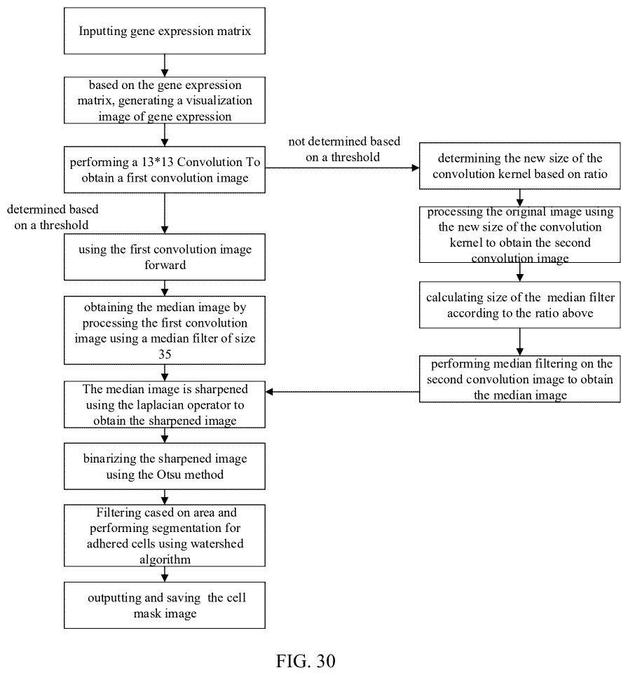

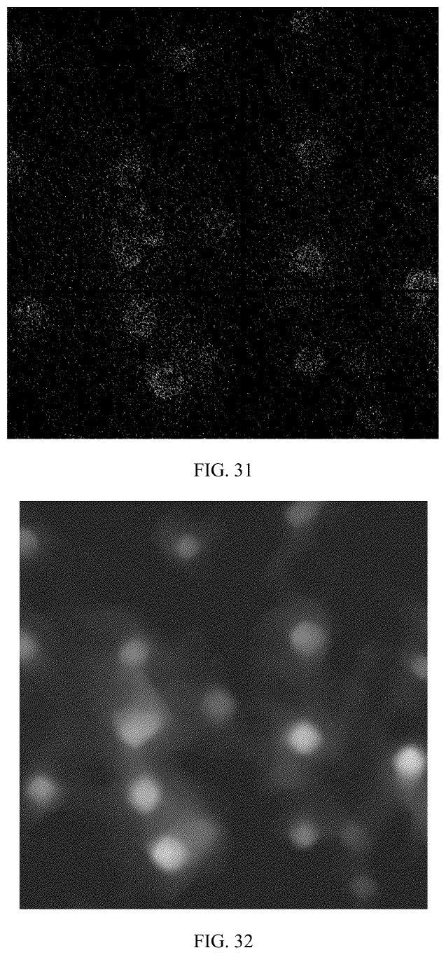

For example, as shown in FIG. 30, a schematic flow diagram of an example of the method according to an embodiment of the present disclosure is shown. The gene expression matrix may first be input, and the visualization image of gene expression may be generated based on the matrix, as shown in FIG. 31. Subsequently, a 13*13 convolution kernel is used to perform a convolution operation on the visualization image of gene expression, so that enabling the scatters in the visualization image of gene expression to be adhered together, thereby obtaining a first convolution image. In this case, a judgment is required with the threshold, in other words, according to the image of the two-dimensional gray peak detection of the first convolution image of the local maximum points, taking out the 99% of all the local maximum points obtained from the quantile value R, if the gap between the value of R and a predetermined empirical threshold is too large (too high or too low, both affect the subsequent processing), then the 13*13 convolution kernel will be considered as being not applicable to the original image, K=13*(13/R) will be calculated and the original 13*13 convolution kernel will be changed to K*K for re-processing the original image, thereby obtaining the second convolution image; if the R value is within the permissible range, then the first convolution image will be used forward to obtain the median image by processing the first convolution image using a median filter of size 35.

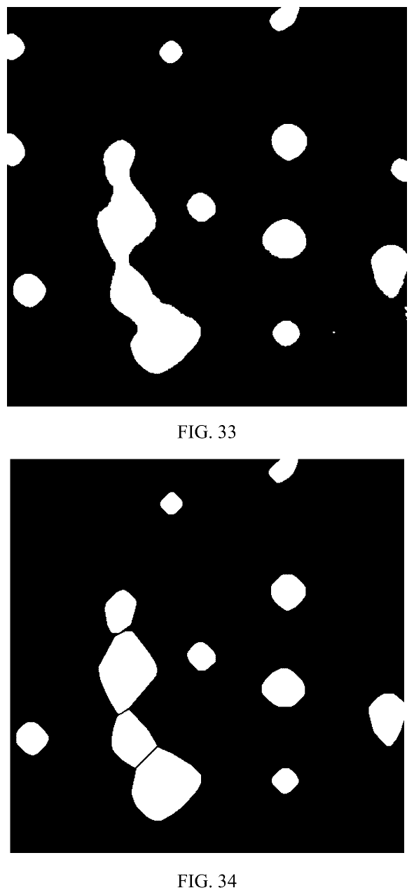

The obtained median image is sharpened using the laplacian operator to obtain the sharpened image, as shown in FIG. 32. The initial mask image is obtained by binarizing the sharpened image using the Otsu's method, as shown in FIG. 33. Then the adherent cells are segmented by area filtering and watershed algorithm. Finally the cell mask image is output and saved, as shown in FIG. 34.

Compared with currently existing cell segmentation methods, the present embodiment provides a scheme for cell segmentation directly based on a visualization image of gene expression, using a combination of multiple image processing methods, which can provide more reliable cell segmentation results. The cell segmentation does not rely on the filmed image and does not require the additional introduction of a technique for registering the filmed image with the visualization image of gene expression, which excludes the introduction of additional errors and saves overall operation time and technical costs, and improve the efficiency and accuracy of the cell segmentation processing.

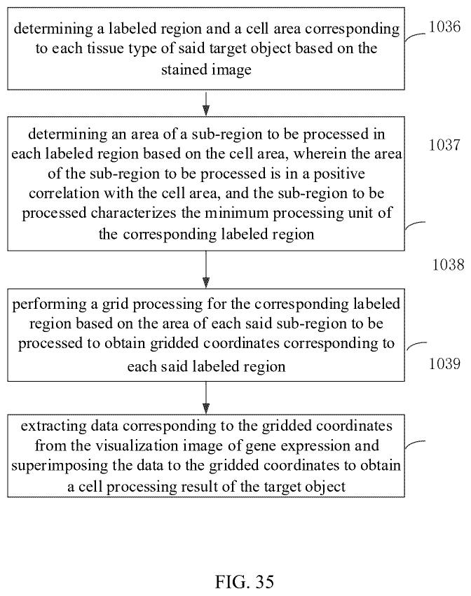

In some embodiments, as shown in FIG. 35, a schematic flow diagram of another method for performing a cell segmentation for a registered image according to an embodiment of the present disclosure, the method includes steps as follows.

Step 1036, determining a labeled region and a cell area corresponding to each tissue type of said target object based on the stained image.

Specifically, the labeled region and the cell area corresponding to each tissue type of said target object are determined based on the stained image, in view of the tissue information provided by the stained image and the target object-related a priori knowledge. The target object-related priori knowledge may be specific cellular included in the target objects, or may be the cellular morphology of those tissues. In an optional embodiment, the tissue boundaries of the individual cellular tissues of the target object may be mapped by a specific manual tool, and the regions corresponding to the different tissues may be labeled by different colors. Among other things, the specific manual tool may be the open source tool 3D slicer, which supports manual labeling, and may also be combined with the open source tool plant-seg network for labeling the tissue regions.

The cell area in this step may be a reference cell area pre-set for the tissue type of the target object. In other words, the cell area for each tissue type is set in advance, and this set cell area may be set empirically or may be set based on an average value of tissue cells of the same type for different objects. Therefore, when the present step is performed, this pre-set cell area can be directly acquired as the cell area corresponding to the labeled area, and subsequent processing can then be performed.

Step 1037, determining an area of a sub-region to be processed in each labeled region based on the cell area, wherein the area of the sub-region to be processed is in a positive correlation with the cell area, and the sub-region to be processed characterizes the minimum processing unit of the corresponding labeled region.

The sub-region to be processed corresponding to the labeled region can be used as the minimum processing unit of the labeled region for processing and analysis of the tissue cells. When extracting positional information of the cells within the target object by using the gene chip, the sub-region to be processed is extracted as a single unit, and the positional relationship between the cells within the labeled region is characterized by the positional relationship between the sub-regions to be processed. Because the area of the sub-region to be processed is in a positive correlation with the cell area. In other words, the larger the cell area of the labeled region determined in the previous step, the larger the sub-region to be processed into which the labeled region is divided. Therefore, the positional relationship between cells in the labeled region corresponding to different tissue types may be expressed better.

Step 1038, performing a grid processing for the corresponding labeled region based on the area of each said sub-region to be processed to obtain gridded coordinates corresponding to each said labeled region.

Optionally, a grid processing for the labeled regions may be performed specifically by dividing a grid with an area size of the corresponding area of the sub-region to be processed in each labeled region separately and obtaining the gridded coordinates.

Optionally, the center point coordinates of the grid may also be used as the gridded coordinates of the grid, wherein the center point coordinates of the grid may be determined by obtaining a coordinate interval of the grid in the coordinates established by the capture unit; calculating the center point coordinates of the coordinate interval; and determining the center point coordinates of the grid.

Step 1039, extracting data corresponding to the gridded coordinates from the visualization image of gene expression and superimposing the data to the gridded coordinates to obtain a cell processing result of the target object.

Specifically, the data corresponding to each gridded coordinates is extracted in the gene expression data to obtain the tissue cell processing data of the target object, which contains the expression levels of different genes of the target object in the cell tissues of each tissue type, and is further combined with the corresponding gridded coordinates to generate the tissue cell processing results of the target object.

In one possible implementation, the gene expression data includes a gene identifier ID, gene coordinate data (x, y), and a gene expression level (MIDCount). Since the gridded coordinates have been determined at this point, the gridded coordinates can be used to determine the coordinate range of each grid. Then the gene coordinate data x, y of each data is compared with the coordinate range of each grid, if the gene coordinate data x, y falls within the coordinate range of any grid, it means that the data is to be added to this grid, at the same time, the data may be set a corresponding grid's ID, i.e., Cell ID, in the expression data. In the subsequent superimposition of data, according to the added Cell ID, it is clear in which grid each data falls, that is, the data needs to be superimposed to the center point coordinates of which grid.

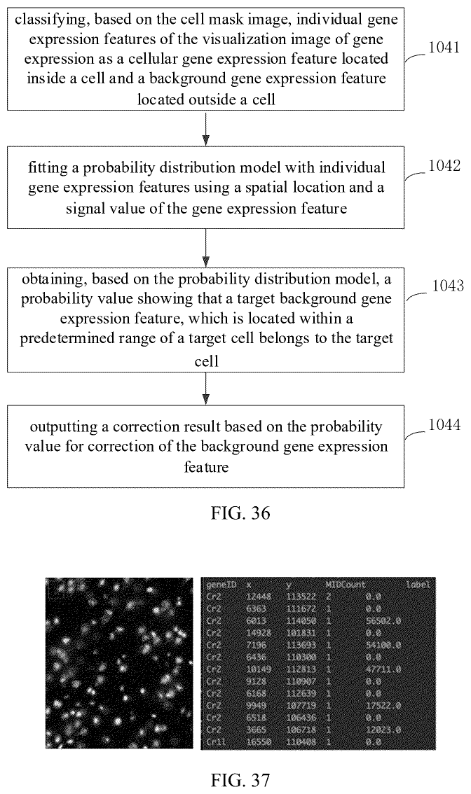

In some embodiments, as shown in FIG. 36, a schematic flow diagram of a method for determining cell-belonging for a background gene molecule unknown of the cell-belonging in the visualization image of gene expression according to an embodiment of the present disclosure, the method include following steps.

Step 1041, classifying, based on the cell mask image, individual gene expression features of the visualization image of gene expression as a cellular gene expression feature located inside a cell and a background gene expression feature located outside the cell.

In this step, based on said cell mask image, each gene expression feature of the gene image is separately identified as a cellular gene expression feature located inside the cell and a background gene expression feature located outside the cell.

In this embodiment, the gene expression feature is DNBs, as shown with reference to FIG. 37, which shows a schematic diagram of a correspondence between a DNB and a cell membrane, wherein x and y are DNB coordinates, respectively, geneID and MIDCount characterize gene information, respectively, and label characterizes cell identity. Each DNB of the Stereo-seq spatial transcriptome data was passed through the correspondence to get the information of the cell to which the DNB belongs, and the rest of the DNBs that are not in the cell were used as background DNBs.

Step 1042, fitting a probability distribution model with individual gene expression features using a spatial location and a signal value of the gene expression feature.

In this step, for each said cellular gene expression signature, a probability distribution model is fitted by the spatial location and the signal value of the gene expression feature.

Preferably, in this embodiment, the probability distribution model may be a Gaussian mixture model, but different probability distribution models may be used. And determining the cell belonging of the background signal, the Bayesian model may be used for the determination. The model may be based on the actual needs of the corresponding selection and adjustment.

In this step, for each intracellular DNB, a Gaussian mixture model is fitted by coordinates (x, y) as well as DNB signal values.

Step 1043, obtaining, based on the probability distribution model, a probability value showing that a target background gene expression feature, which is located within a predetermined range of a target cell belongs to the target cell.

In this step, based on a probability distribution model, a probability value is determined for a target background gene expression feature located within a predetermined range for a target cell belonging to the cell. The target cells may be all cells included in the biological sample or may be some of the cells included in the biological sample.

Optionally, specifically in this step, taking the target cell a as cell a for example, for cell a, a probability value of the background DNB belonging to the cell a is calculated by a Gaussian mixture model within a range of 50×50 pixel points centered on the cell a.

Step 1044, outputting a correction result based on the probability value for correction of the background gene expression feature.

Correction of background gene expression features is based on probability values.

Specifically, in this step, the probability value is determined that whether the probability value is greater than or equal to the predetermined probability value, and if so, the target background gene expression feature is treated as a cellular gene expression feature located within the target cell for correction of the background gene expression feature, and if not, the target background gene expression feature continues to be treated as the background gene expression feature.

With reference to FIG. 38, a schematic diagram of a correction result of correcting a background DNB, for the background DNB, the background DNB whose probability value is less than the predetermined probability value is treated as the background DNB, and the background DNB whose probability value is greater than or equal to the predetermined probability value are treated as the DNB of the cell.

In this embodiment, the predetermined probability value is not specifically limited, and can be selected and adjusted accordingly based on actual needs.

As an optional embodiment, in this step, in response to the target background gene expression feature being located outside of the at least two target cells at the same time, a probability value belonging to each corresponding target cell is separately determined; the highest probability value from the at least two probability values determined is selected as the probability value for outputting the correction result, and the target cell corresponding to the highest probability value is taken as the cell to which the target background gene expression feature belonging.

Taking the target cells as cell a and cell b as an example, assuming that for the background gene expression feature A is within the center range of 50×50 pixel points of cell a and cell b at the same time, the probability value that the background gene expression feature A belongs to cell a is calculated as x1, and that the probability value that the background gene expression feature A belongs to cell b is calculated as x2, and if x1 is greater than x2, then the background gene expression feature A is determined as the cell a's gene expression feature.

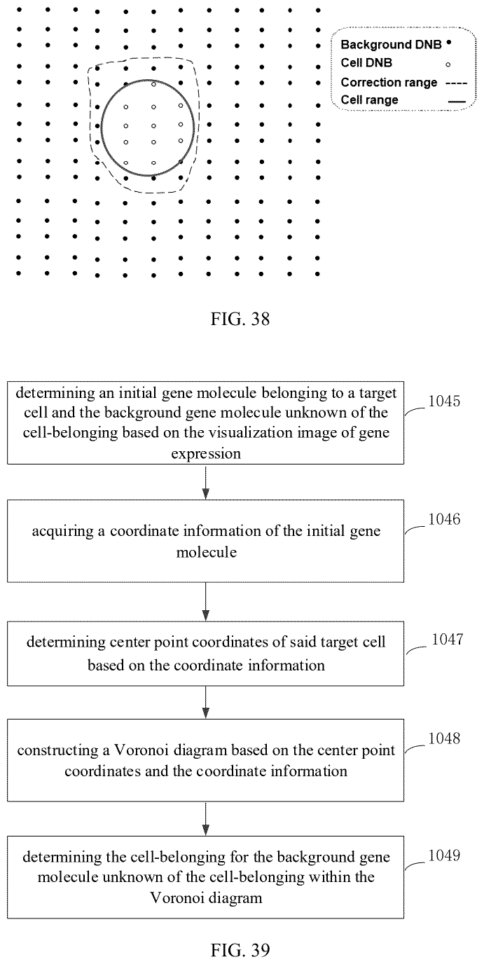

In some embodiments, as shown in FIG. 39, a schematic flow diagram of another method for determining cell-belonging for a background gene molecule unknown of the cell-belonging in the visualization image of gene expression according to an embodiment of the present disclosure, the method includes following steps.

Step 1045, determining an initial gene molecule belonging to a target cell and the background gene molecule unknown of the cell-belonging based on the visualization image of gene expression.

Step 1046, acquiring a coordinate information of the initial gene molecule.

The microscope image and the gene image are aligned according to the coordinates, and the coordinate information of the initial gene molecules in the cell can be obtained by the cell segmentation boundary of the microscope image, i.e., the coordinate information of the initial gene molecules in the cell can be obtained.

Step 1047, determining center point coordinates of said target cell based on the coordinate information.

Step 1048, constructing a Voronoi diagram based on the center point coordinates and the coordinate information.

The Voronoi diagram (also known as the Tyson polygon or Dirichlet diagram) consists of a set of contiguous polygons, which consist of perpendicular bisectors of straight lines connecting two neighboring points. Specifically, there are N distinct points on the plane, and the plane is divided according to the principle of closest proximity, each point is associated with its nearest neighboring region.

Step 1049, determining the cell-belonging for the background gene molecule unknown of the cell-belonging within the Voronoi diagram.

Based on the labels being set for the background gene molecules within the Voronoi diagram, in can determine that the background gene molecules within the Voronoi diagram belong completely to the target cell, belong partially to the target cell, or not belong to the target cell at all.

In the present embodiment, the coordinates of the center point coordinates of the target cell are determined by the coordinate information of the initial gene molecules belonging to the target cell; and then a Voronoi diagram is constructed based on the coordinates of the center point of the target cell; and the cell-belonging for the background gene molecule within the range of the Voronoi diagram are determined, thus improving the efficiency and accuracy of the correction. In addition, according to the present embodiment, only the initial gene molecules in the target cell need to be determined, and the boundary image of the target cell is unnecessary, thus reducing the requirement for high accuracy of cell segmentation, reducing the degree of dependence on the morphological image map of the target cell, and improving the robustness of the correction scheme.

In some embodiments, said step of determining a cell-belonging for a background gene molecule unknown of the cell-belonging in the visualization image of gene expression based on the cell mask image includes:
obtaining a microscope image and a gene image of biological sample;
performing image registration for the microscope image and the gene image of biological sample;
performing cell segmentation on said microscope image to obtain cell segmentation results;
determining the gene image of a target cell based on said cell segmentation results and image registration results of said gene image;
based on the gene image of said target cell, determining initial gene molecules belonging to the target cell and background gene molecules unknown cell-belonging.

In some embodiments, said step of determining center point coordinates of said target cell based on the coordinate information includes:
acquiring the number of molecules of the initial gene molecules;
calculating the sum of the horizontal coordinate values and the sum of the vertical coordinate values of said initial gene molecule;
determining a first ratio as a value of the horizontal coordinates of said center point coordinates, wherein said first ratio is the ratio of the sum of the values of the horizontal coordinates of said initial gene molecules to the number of said molecules;
determining the second ratio as a vertical coordinate value of said center point coordinates, wherein said second ratio is the ratio of the sum of the vertical coordinate values of said initial gene molecules to said number of molecules.

In some embodiments, said step of determining center point coordinates of said target cell based on the coordinate information includes:
acquiring the maximum and minimum horizontal coordinate value, the maximum and minimum vertical coordinate value
determining a third ratio as a horizontal coordinate value of said center point coordinates, wherein said third ratio is one-half of the difference between the maximum horizontal coordinate value and the minimum horizontal coordinate value;
determining a fourth ratio as a vertical coordinate value of said center point coordinates, wherein said fourth ratio is one-half of the difference between the maximum vertical coordinate value and the minimum vertical coordinate value.

In some embodiments, the method of gene image data correction further includes:
if said background gene molecules are within ranges of different Voronoi diagram, said background gene molecules are input into a Gaussian mixture model to determine the probability scores that said background gene molecules belongs to a target cell within the predetermined distance range, respectively;
correcting said background gene molecules as gene molecules belonging to target cells with higher probability score.

In some embodiments, after said step of determining the cell-belonging for the background gene molecule unknown of the cell-belonging within the Voronoi diagram, the method includes:
generating gene expression information of the target cell based on the corrected results of gene molecules belonging to said target cell.

In some embodiments, said step of constructing a Voronoi diagram based on the center point coordinates and the coordinate information includes:
constructing polygonal net for all said center point coordinates;
determining the edges of the Voronoi diagram based on said polygonal net;
constructing a Venno graph based on the edges of said Venno diagram.

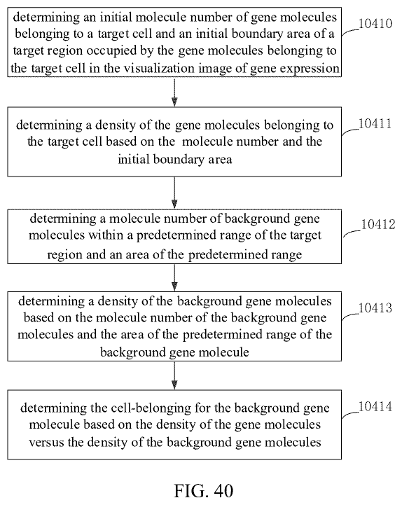

In some embodiments, as shown in FIG. 40, a schematic flow diagram of another method for determining cell-belonging for a background gene molecule unknown of the cell-belonging in the visualization image of gene expression according to an embodiment of the present disclosure, the method includes steps as follows.

Step 10410, determining an initial molecule number of gene molecules belonging to a target cell and an initial boundary area of a target region occupied by the gene molecules belonging to the target cell in the visualization image of gene expression.

The initial boundary area of the target region occupied by the gene molecules belonging to the target cell is determined using the Convex Hull Algorithm, which operates on the cell segmentation results. In this case, the Convex Hull Algorithm finds the convex hull of N points in the target region according to the cell segmentation results, and the initial boundary area is surrounded by the connection between these N points.

Step 10411, determining a density of the gene molecules belonging to the target cell based on the molecule number and the initial boundary area.

In an optional embodiment, the ratio of the number of molecules to the initial boundary area is determined as the density of gene molecule of the target cell.

Step 10412, determining a molecule number of background gene molecules within a predetermined range of the target region and an area of the predetermined range.

The background gene molecules are gene molecules whose cell-belonging cannot be identified based on the gene image, and the predetermined region range is located outside the target region, but cannot exceed the area occupied by gene molecules belonging to neighboring cells. An optional region area of 5 pixels*5 pixels is used as the predetermined region range.

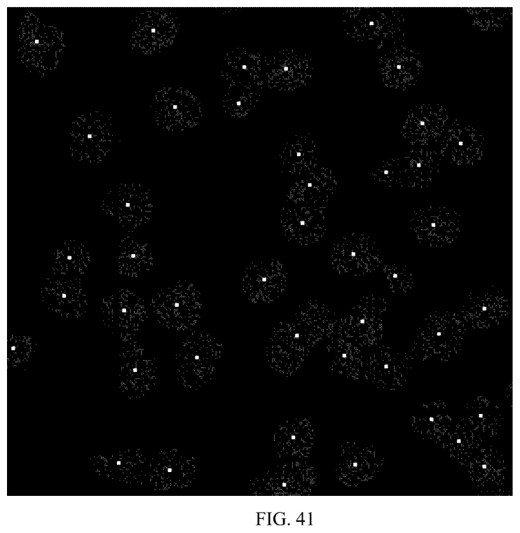

In an optional embodiment, the predetermined range of regions may be a region centered on a region determined based on a center point of the target cell. The center point of the target cell is determined from the genetic image. See FIG. 41, a schematic diagram of a center point of a target cell.

Step 10413, determining a density of the background gene molecules based on the molecule number of the background gene molecules and the area of the predetermined range of the background gene molecule.

In an optional embodiment, the ratio of the number of molecules of the background gene molecule to the area of the region is determined as the background gene molecule density.

Step 10414, determining the cell-belonging for the background gene molecule based on the density of the gene molecules versus the density of the background gene molecules.

In this embodiment, utilizing the above-described cell characteristics, the density of the gene molecule of the target cell is determined based on an initial number of molecules of a gene molecule belonging to the target cell in the gene image versus an initial boundary area of the target cell. The density of gene molecule density of the target cell is compared with that of background gene molecule, and the cell-belonging for the background gene molecule is determined based on the density of the initial gene molecules belonging to the target cell versus the density of the background gene molecules. Therefore, the efficiency and the accuracy of correcting the gene molecules in the target cell is substantially increased.

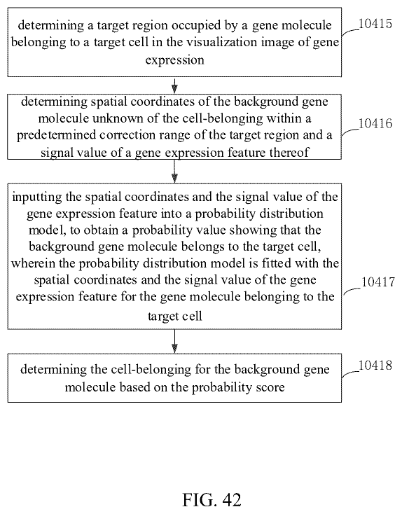

In some embodiments, as shown in FIG. 42, a schematic flow diagram of another method for determining cell-belonging for a background gene molecule unknown of the cell-belonging in the visualization image of gene expression according to an embodiment of the present disclosure, the method includes steps as follows.

Step 10415, determining a target region occupied by a gene molecule belonging to a target cell in the visualization image of gene expression.

Step 10416, determining spatial coordinates of the background gene molecule unknown of the cell-belonging within a predetermined correction range of the target region and a signal value of a gene expression feature thereof.

In this embodiment, the gene expression features are DNBs, and each DNB of the Stereo-seq spatial transcriptome data can be used to obtain the cell belonging information of the cell to which the DNB belongs, by correspondence. The remaining of the DNBs that are not within the cell are used as the background DNBs. The spatial coordinates of the background gene molecules can be expressed as the coordinates of the background DNBs.

Step 10417, inputting the spatial coordinates and the signal value of the gene expression feature into a probability distribution model, to obtain a probability value showing that the background gene molecule belongs to the target cell, wherein the probability distribution model is fitted with the spatial coordinates and the signal value of the gene expression feature for the gene molecule belonging to the target cell.

When fitting a GMM model (Gaussian Mixture Model), there are three independent variables: spatial coordinates (x, y) and signal values. The dependent variable, i.e. the output, is the probability score of each gene molecule belonging to the target cell. The specific implementation process includes: introducing Gaussian Mixture in Scikit-learn library (a kind of machine learning library); and automatically fitting the parameter information of the GMM model using the input data to obtain the final probability model.

The probability distribution model is fitted by the spatial coordinates of the sample of gene molecules belonging to the target cell, and the sample of gene expression feature signal values. As a preferred embodiment, in this embodiment, the probability distribution model may be a Gaussian mixture model.

Step 10418, determining the cell-belonging for the background gene molecule based on the probability score.

The probability score is compared to determine whether the probability score is greater than or equal to the probability threshold.

If the probability score is greater than or equal to a probability threshold, correcting the background gene molecule as a gene molecule belonging to the target cell. If the probability score is less than the probability threshold, the background gene molecule remains a background molecule with the unknown cell-belonging.

In an optional implementation, a third quartile between the first probability score and the second probability score is determined as a probability threshold.

The first probability score is the probability score with the highest score value among the probability scores; and the second probability score is the probability score with the lowest score value among the probability scores.

In this embodiment, under the premise of roughly knowing the target region of the target cell, a probability score that background molecule with unknown cell-belonging in predetermined correction range belongs to the target cell is calculated. And the cell-belonging of the background molecule is determined based on the probability score. The situation in which the gene image data has a large error due to the diffusion of the gene molecules is reduced, and the correction efficiency is improved. It also does not depend on the high-precision of cell segmentation results, which enhances the robustness of the correction scheme.

Further, the present disclosure also provides detection of track crosses on the chip, primarily for subsequent corrections of stitching and registration. Both splicing and stitching are useful for the detection of the TRACK.

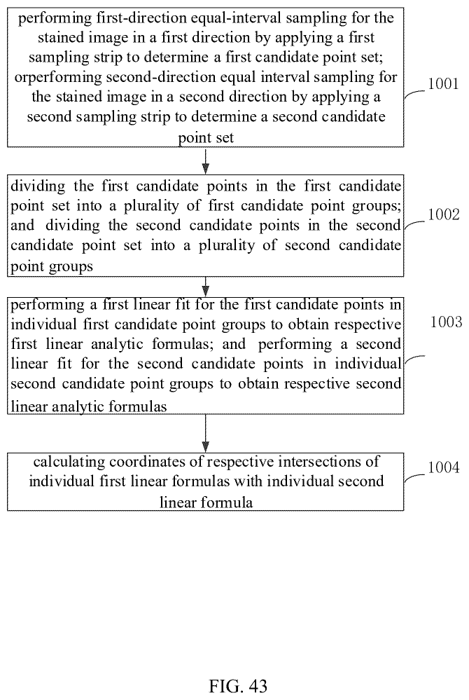

FIG. 43 illustrates a schematic flow diagram of a method 100 for determining coordinates of the point in the stained image according to an embodiment of the present disclosure. Said method 100 may include a sampling step 1001, a grouping step 1002, a linear fitting step 1003, and a coordinate calculation step 1004.

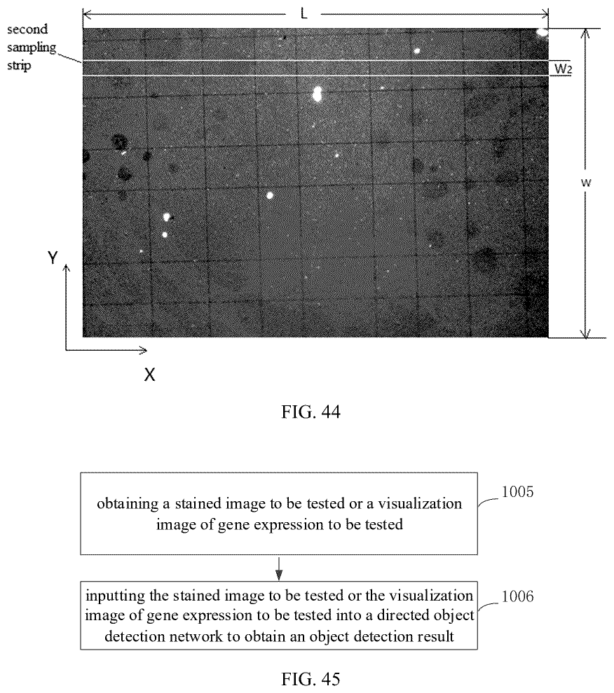

In order to facilitate an understanding of the determining method of the present disclosure, the stained images involved herein are now illustrated with reference to FIG. 44. FIG. 38 illustrates a schematic diagram of the stained image according to an embodiment of the present disclosure. The stained image may be acquired by a camera, a stained image sensor, and the like. The stained image may be a stained image of, for example, a mechanical structure, a carrier, a slide, or a biochip having straight track lines. As illustrated in FIG. 38, the plane where the stained image is located may include a first direction X and a second direction Y perpendicular to the first direction X, the stained image may include a plurality of first straight track lines transverse to the second direction Y (i.e., a plurality of dark lines in the stained image that are substantially parallel to the first direction X) and a plurality of second straight track lines transverse to the first direction X (i.e., a plurality of dark lines in the stained image that are substantially parallel to the second direction Y). Specifically, there are six first straight track lines and eight second straight track lines in the exemplary stained image. Since each of the first straight track lines intersects each of the second straight track lines, there are a total of 6×8=48 intersection points in the exemplary stained image. As illustrated in FIG. 38, the stained image may have a pixel length L and a pixel width W. Also illustrated in FIG. 38 is an exemplary second sampling strip. By way of illustration and not limitation, the pixel length of the sampling strip is the same as the pixel length of the example stained image, i.e., L, and the pixel width of the sampling strip is smaller than the pixel width of the example stained image, i.e., W2. Herein, a straight track line being transverse to the any direction means that the straight track line is parallel to the direction that is perpendicular to the either direction, or means that the angle between said any direction is less than a predetermined value. Said predetermined value may range from 1 to 10 degrees, from 10 to 20 degrees, from 20 to 30 degrees, from 30 to 40 degrees, or from 40 to 50 degrees, etc., for example, said predetermined value may be 1 degree, 2 degrees, 3 degrees, 4 degrees, 5 degrees, 6 degrees, 7 degrees, 8 degrees, 9 degrees, 10 degrees.

The sampling step 1001 includes a first direction sampling step and a second direction sampling step.

In a first direction sampling step, performing first-direction equal-interval sampling for the stained image in a first direction by applying a first sampling strip to determine a first candidate point set, wherein for each time of said first-direction equal-interval sampling, the following first direction sub-sampling step one and first direction sub-sampling step two are performed. The first direction sub-sampling step one includes: calculating a cumulative sum of pixel values in a first direction for pixels within a sampled region of the stained image selected during this time of said first-direction equal-interval sampling by the first sampling strip, to obtain a series of first pixel value sums, wherein said series of first pixel values sums at any second direction position in said second direction is equal to the pixel values sums of each pixel values registered along the first direction at said any second direction position within said sampling region. The first direction sub-sampling step two includes: determining a first extreme point of the series of first pixel value sums, and determining, for each first extreme point determined, a first candidate point obtained from this time of said first-direction equal-interval sampling, wherein a coordinate of the first candidate point in the first direction is determined according to a location of the first sampling strip in the first direction during this time of said first-direction equal-interval sampling, a coordinate of the first candidate point in the second direction is determined according to a coordinate in the second direction corresponding to a location of the first extreme point in the second direction. In this way, the first candidate point set consists of the first candidate points obtained from individual times of said first-direction equal interval sampling.

In a second direction sampling step, performing second-direction equal interval sampling for the stained image in a second direction by applying a second sampling strip to determine a second candidate point set, wherein for each second direction equal interval sampling, the following second direction sub-sampling step one and second direction sub-sampling step two are performed. The second direction sub-sampling step one includes: calculating a cumulative sum of pixel values in a second direction for pixels within a sampled region of the stained image selected during this time of said second-direction equal-interval sampling by the second sampling strip, to obtain a series of second pixel value sums, wherein said series of second pixel values sums at any first direction position in said first direction is equal to the pixel values sums of each pixel values registered along the second direction at said any first direction position within said sampling region. The second direction sub-sampling step includes: determining a second extreme point of the series of second pixel value sums, and determining, for each second extreme point determined, a second candidate point obtained from this time of said second-direction equal interval sampling, wherein a coordinate of the second candidate point in the second direction is determined according to a location of the second sampling strip in the second direction during this time of said second-direction equal-interval sampling, a coordinate of the second candidate point in the first direction is determined according to a coordinate in the first direction corresponding to a location of the second extreme point in the first direction. In this way, the second candidate point set consists of the second candidate points obtained from individual times of said second-direction equal interval sampling.

All extreme value points used in the sampling step 1001 may be the same extreme value points or the same extreme value points. For example, if said straight track line is a dark line, all extreme value points used may be the same extremely small value points; if said straight track line is a bright line, all extreme value points used may be the same extremely large value points.

In the grouping step 1002, dividing the first candidate points in the first candidate point set into a plurality of first candidate point groups; and dividing the second candidate points in the second candidate point set into a plurality of second candidate point groups.

In one embodiment, the first candidate points in the first candidate point set is divided into a plurality of first candidate point groups in the first grouping approach, wherein said first grouping approach is configured such that respective first candidate points in each first candidate point group is arranged substantially in a straight line transverse to said second direction. And, the second candidate points in the second candidate point set are divided into a plurality of second candidate point groups in a second grouping approach, wherein said second grouping approach is configured such that respective second candidate points in each second candidate point group is arranged substantially in a straight line transverse to said first direction.

In the linear fitting step 1003, performing a first linear fit for the first candidate points in individual first candidate point groups to obtain respective first linear analytic formulas; and performing a second linear fit for the second candidate points in individual second candidate point groups to obtain respective second linear analytic formulas.

Performing the linear fitting based on a plurality of points with known coordinates to obtain linear analytic formulas is known in the art and will not be repeated herein. Commonly used linear fitting methods include: least squares; gradient descent; and Gaussian Newton, Levenberg-Marquardt.

In the coordinate calculation step 1004, calculating coordinates of respective intersections of individual first linear formulas with individual second linear formula. Methods for finding the coordinates of the intersection of two known linear analytic formulas are known in the art and will not be repeated herein.

FIG. 45 shows a schematic flow diagram of a directed object detection method according to an embodiment of the present disclosure, the method includes steps as follow.

Step 1005, obtaining a stained image to be tested or a visualization image of gene expression to be tested.

Step 1006, inputting the stained image to be tested or the visualization image of gene expression to be tested into a directed object detection network to obtain an object detection result.

Specifically, the stained image to be tested or the visualization image of gene expression to be tested is input into the trained YOLOV6-based directed object detection model, and the trained multilevel convolutional neural network in the directed object detection model is utilized to perform convolutional operation and fusion of feature image on the image to be detected, and feature fusion images at different scales are output; for the feature fusion images at different scales, the corresponding predictors are used to predict the categorization confidence, localization confidence for object box, object detection confidence and angle classification confidence.

Said Backbone layer utilizes a RepVGG architecture and said Neck layer utilizes a FPN-PAN architecture.

Said head layer employs a decoupling head such that a category analysis is performed via a categorization branch for obtaining said categorization information, and a regression analysis is performed via a regression branch for obtaining said object box coordinate prediction information, said object prediction information, and said angle prediction information, respectively. Described are the categorization information for representing the result of the categorization analysis, the object box coordinate prediction information for determining the position of the object detection box, the object prediction information for determining whether a corresponding object exists in the object detection box, and the angle prediction information for determining the angle of the object detection box, and wherein the corresponding object may include a track line or a track cross (also referred to as a track intersection). Embodiments of the present disclosure provide a method for training a YOLOV6-based directed object detection network, including: obtaining a plurality of training images and labeling information, including labels, of said plurality of training images; and inputting said plurality of training images and said labeling information into said YOLOV6-based directed object detection network to obtain setup parameters for said YOLOV6-based directed object detection network by training. Said YOLOV6-based directed object detection network includes: a backbone layer configured to perform a feature extraction on the stained image and the visualization image of gene expression, to obtain a plurality of feature images at different scales; a neck layer configured to receive the plurality of feature images from the backbone layer and perform a cross-scale fusion for the plurality of feature images, to obtain a plurality of fused feature images; and a detection head layer configured to perform a prediction for the plurality of fused feature images, to obtain the object detection result with respect to the stained image to be tested or the visualization image of gene expression to be tested, wherein the object detection result incudes: a prediction information of an object box coordinate, an object prediction information and an angle prediction information.

In some embodiments, said Backbone layer utilizes a RepVGG architecture and said Neck layer utilizes an FPN-PAN architecture.

In some embodiments, said Head layer employs a decoupling head such that a category analysis is performed via a categorization branch for obtaining said categorization information, and a regression analysis is performed via a regression branch for obtaining said object box coordinate prediction information, said object prediction information, and said angle prediction information, respectively.

In some embodiments, said step of inputting said plurality of training images and said labeling information into said YOLOV6-based directed object detection network to obtain setup parameters for said YOLOV6-based directed object detection network by training includes: in each time of training,
obtaining the corresponding training image and corresponding label information for the current training correspondingly;
predicting an object in said corresponding training image using said YOLOV6 based
directed object detection network to obtain corresponding prediction results;
calculating the loss function of said YOLOV6-based directed object detection network based on the corresponding label information and the corresponding prediction results; and
based on the calculation results of said loss function, adjusting setup parameters of said YOLOV6-based directed object detection network until the calculation results of said loss function converge.

In some embodiments, the method further includes: pre-processing the obtained labeling information having the form of the first data, to process said labeling information into data in the form of [x, y, w, h, θ, classID], and inputting the processed data into said YOLOV6-based directed object detection network.

The (x, y) denotes the position of the center of the object detection box, w denotes the width of the long side of said object detection box, h denotes the height of the short side of said object detection box, θ denotes the angle at which the long side of said object detection box is rotated with respect to the horizontal axis, and classID denotes the category of said object detection box.

In some embodiments, the first data form of said labeling information is [x1, y1, x2, y2, x3, y3, x4, y4, classID], wherein (x1, y1), (x2, y2), (x3, y3), and (x4, y4) define the positions of the four vertexes of the object detection box, respectively.

In some embodiments, the method further includes: post-processing the obtained prediction results in the form of [x, y, w, h, θ, conf, classID], to process said prediction results into data having the form of the second data, and presenting said data having the form of the second data to the user.

The (x, y) denotes the position of the center of the object detection box, w denotes the width of the long side of said object detection box, h denotes the height of the short side of said object detection box, θ denotes the angle at which the long side of said object detection box is rotated with respect to the horizontal axis, conf denotes the confidence that said object detection box has the corresponding target, and classID denotes the category of said object detection box.

In some embodiments, said second data is in the form of [x1, y1, x2, y2, x3, y3, x4, y4, conf, classID], wherein (x1, y1), (x2, y2), (x3, y3), and (x4, y4) define the position of each of said four vertexes of said object detection box, respectively.

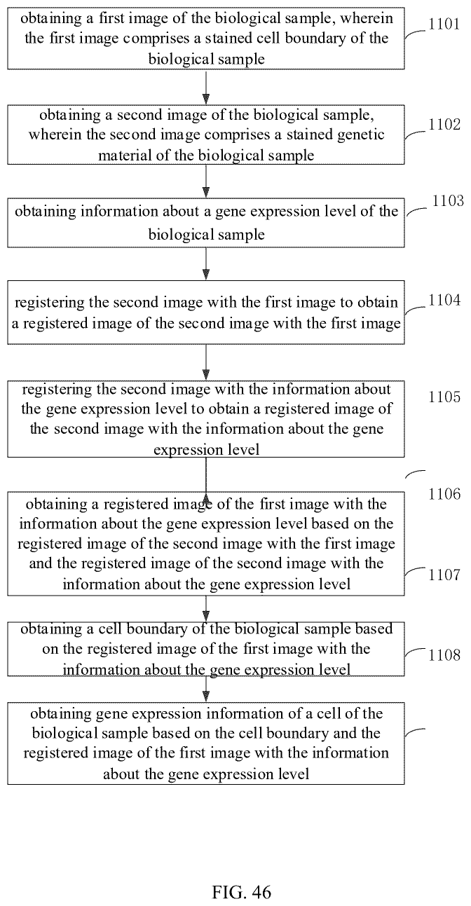

Further, as shown in FIG. 46, a schematic flow diagram of a method for obtaining gene expression information of a cell of the biological sample according to an embodiment of the present disclosure, the method includes:
step 1101, obtaining a first image of the biological sample, wherein the first image includes a stained cell boundary of the biological sample;
step 1102, obtaining a second image of the biological sample, wherein the second image includes a stained genetic material of the biological sample;
step 1103, obtaining information about a gene expression level of the biological sample;
step 1104, registering the second image with the first image to obtain a registered image of the second image with the first image;
step 1105, registering the second image with the information about the gene expression level to obtain a registered image of the second image with the information about the gene expression level;
step 1106, obtaining a registered image of the first image with the information about the gene expression level based on the registered image of the second image with the first image and the registered image of the second image with the information about the gene expression level;
step 1107, obtaining a cell boundary of the biological sample based on the registered image of the first image with the information about the gene expression level;
step 1108, obtaining gene expression information of a cell of the biological sample based on the cell boundary and the registered image of the first image with the information about the gene expression level.

In some embodiments, the cells of the biological sample are stained with an ssDNA staining solution and a cell boundary fluorescent staining solution, wherein said ssDNA staining solution includes a buffer and an ssDNA reagent, and said cell boundary fluorescent staining solution includes a fluorescent dye. Said ssDNA staining solution may be used to perform staining to the genetic material of the cells, and the cell boundary fluorescent staining solution may be used to perform staining to the boundaries of the cells.

In some embodiments, said cell boundary includes a cell wall or a cell membrane.

In some embodiments, gene expression information for a cell of said tissue sample includes information about the location, the gene expression level, and the cell type of each sites on a chip containing said tissue sample.

In some embodiments, said step of registering the second image with the information about the gene expression level to obtain a registered image of the second image with the information about the gene expression level includes:

registering said second image and said information about the gene expression level based on track lines in said second image and track lines of said information about the gene expression level, to obtain a registered image of said second image with the information about the gene expression level.

In some embodiments, imaging the tissue sample after staining tissue sample by a cell staining reagent, said imaging includes acquiring the second image of said tissue sample via a FITC channel and the first image of said tissue sample via a DAPI channel, said cell staining reagent includes the ssDNA staining solution and the cell boundary fluorescent staining solution; said ssDNA staining solution includes a buffer and an ssDNA reagent, and said cell boundary fluorescent staining solution includes a fluorescent dye.

It should be noted that the above numerical order of the steps does not represent specific steps of execution.

Corresponding to the above-described method for obtaining spatial omics single cell data, the present disclosure also proposes a device for obtaining spatial omics single cell data. Since the device embodiment of the present disclosure corresponds to the above-described embodiments of method, undisclosed details of the embodiments of device can be referred to the above-described embodiments of method and will not be repeated in the present disclosure.

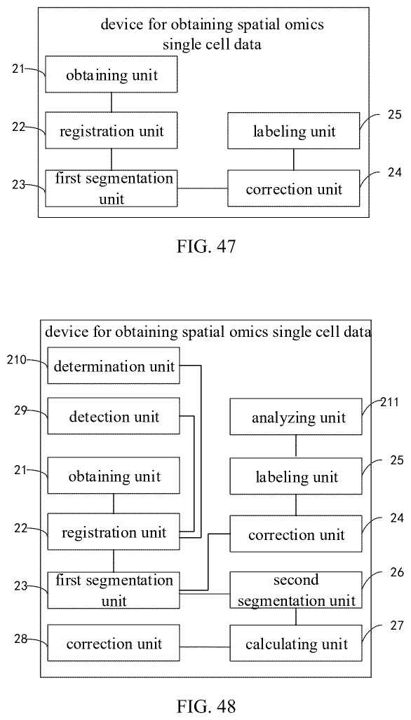

FIG. 47 shows a schematic structural diagram of a device for obtaining spatial omics single cell data according to an embodiment of the present disclosure, as shown in FIG. 47, the device includes:
an obtaining unit 21 for obtaining a stained image of a biological sample and a visualization image of gene expression of the biological sample;
a registration unit 22 for performing image registration for the stained image and the visualization image of gene expression to obtain a registered image;
a first segmentation unit 23 for performing cell segmentation for the registered image to obtain a cell mask image;
a correction unit 24 for determining cell-belonging for a background gene molecule unknown of the cell-belonging in the visualization image of gene expression based on the cell mask image; and
a labeling unit 25 for labeling a cell with the determined cell-belonging for the background gene molecule, to obtain an expression matrix of spatial omics molecules with the cell label.

The present embodiment may obtain a stained image of a biological sample and a visualization image of gene expression of the biological sample; perform image registration for the stained image and the visualization image of gene expression to obtain a registered image; perform cell segmentation for the registered image to obtain a cell mask image; and determine cell-belonging for a background gene molecule unknown of the cell-belonging in the visualization image of gene expression based on the cell mask image, and labeling a cell with the determined cell-belonging for the background gene molecule, to obtain an expression matrix of spatial omics molecules with the cell label. Compared with the related art, the embodiments of the present disclosure realize the obtaining of highly accurate spatial single-cell data by the processing step of gene expression matrix image processing, cropping, registration, segmentation and correction.

Further, in a possible implementation of the present embodiment, as shown in FIG. 48, said obtaining unit 21 is further used for:
obtaining the stained image of the biological sample with a microscopy; or
obtaining a stitched stained image of the biological sample with a microscope, wherein the stitched stained image is stitched from a plurality of sub-stained images; or
obtaining a plurality of sub-stained images of the biological sample with a microscopy, and stitching the plurality of sub-stained images to obtain the stitched stained image.

Further, in a possible implementation of the present embodiment, as shown in FIG. 48, said obtaining unit 21 includes:
a first obtaining module for obtaining the plurality of sub-stained images of the biological sample with a microscopy and performing first stitching for the plurality of sub-stained images of the biological sample to obtain a first stained image;
a determining module for determining whether to perform second stitching for the plurality of sub-stained images of the biological sample based on an image quality of the first stained image;
an identifying module for identifying the first stained image as the stained image in response to a negative result of the determination; and
a second obtaining module for performing the second stitching for the plurality of sub-stained images of the biological sample to obtain the stained image in response to a positive result of the determination.

Further, in a possible implementation of the present embodiment, as shown in FIG. 48, said first obtaining module is further used for:
determining an overlapping region between each of the plurality of sub-stained images and a template image;
obtaining a plurality of overlapping sub-image pairs by matching a plurality of overlapping sub-stained images and a plurality of overlapping sub-template images, wherein a section corresponding to the determined overlapping region in each of the plurality of sub-stained images and a section corresponding to the determined overlapping region in the template image are cropped respectively, to provide the plurality of overlapping sub-stained images and the plurality of overlapping sub-template images that are identical by numbers;
obtaining an offset between each of the plurality of sub-stained images and the corresponding sub-template image by performing frequency domain calculations on the plurality of overlapping sub-image pairs; and
translating each of the plurality of sub-stained images by the offset toward the corresponding sub-template image, and stitching the plurality of sub-stained images together with the template image.

Further, in a possible implementation of the present embodiment, as shown in FIG. 48, said first obtaining module is further used for:
obtaining the plurality of sub-stained images and template information about a track line or a track cross for the biological sample;
performing a pre-stitching for the plurality of sub-stained images to obtain respective pre-stitched coordinates of the plurality of sub-stained images;
selecting at least one first reference image from the plurality of sub-stained images according to a feature of the track line or the track cross;
deriving a global template according to the template information of the biological sample and the pre-stitched coordinates of the first reference image;
for a second reference image in the plurality of sub-stained images, calculating an offset between pre-stitched coordinates of the second reference image and corresponding template coordinates of the second reference image in the global template, wherein second reference image is different from the first reference image; and
adjusting the coordinates of the second reference image by the offset and stitching the second reference image for generation of the stitched stained image.

Further, in a possible implementation of the present embodiment, as shown in FIG. 48, said registration unit 22 includes:
an obtaining module for obtaining a track line in the stained image and rotating the stained image in an angle between the track line and a horizontal direction, wherein the stained image and the visualization image of gene expression each include a plurality of track lines and/or a plurality of track crosses, two of the track lines intersect to form the track crosses, and each of the plurality of track lines is corresponded with an index number;
a processing module for obtaining a third reference image by scaling the stained image, wherein the third reference image has the same scale as the visualization image of gene expression;
a first determining module for determining an object offset based on the visualization image of gene expression and the third reference image; and
a registration module for translating the third reference image by the object offset to obtain the registered image.

Further, in a possible implementation of the present embodiment, as shown in FIG. 48, said registration unit 22 further includes:
a second determining module for determining a similarity between the third reference image and the visualization image of gene expression; and
a coarse registration taking a position with a maximum similarity between the third reference image and the visualization image of gene expression as a result of a coarse registration.

Further, in a possible implementation of the present embodiment, as shown in FIG. 48, said registration unit 22 is further used for:
obtaining the stained image and the visualization image of gene expression each including a plurality of track lines and/or a plurality of track crosses, two of the track lines intersect to form the track crosses, and each of the plurality of track lines is corresponded with an index number;
performing a preprocessing for the stained image to obtain a candidate image set;
determining a binary image set corresponding to the candidate image set;
determining a track cross pair(s) having a correspondence between the binary image set and the visualization image of gene expression, based on a similarity between the binary image set and the visualization image of gene expression;
determining an offset information based on the track cross pair; and
processing an object image based on the offset information to obtain the registered image, wherein the object image is an image from the candidate image set and corresponding to the track cross pair.

Further, in a possible implementation of the present embodiment, as shown in FIG. 48, said device further includes:
a second segmentation unit 26 for performing a tissue segmentation for the registered image to obtain a tissue mask image, before said performing a cell segmentation for the registered image to obtain a cell mask image.

Further, in a possible implementation of the present embodiment, as shown in FIG. 48, said second segmentation unit 26 is also used for:
obtaining a gene expression of an object tissue to be segmented in the registered image to determine a gene expression matrix of the registered image, wherein the gene expression in the gene expression matrix is arranged according to a pixel position;
determining image data by assigning the gene expression at each pixel position in the gene expression matrix to a corresponding pixel position in the registered image;
performing an image processing for the image data to determine a foreground pixel point of the image data; and
labeling connected components for the image data based on the foreground pixel point to obtain the tissue mask image.

Further, in a possible implementation of the present embodiment, as shown in FIG. 48, said device further includes:
a calculating unit 27 for obtaining an outer boundary image and an inner boundary image in a grayscale image corresponding to the tissue mask image by calculating respectively;
a matching unit 28 for dividing the outer boundary image and the inner boundary image into N of segments respectively, and matching each segment of the outer boundary image with each segment of the inner boundary image by pair to obtain N image combinations, wherein each of the image combinations includes one segment of the outer boundary image and one segment of the inner boundary image;
the calculating unit 27 is further used for calculating a ratio of pixel density between one segment of the outer boundary image and one segment of the inner boundary image in each of the image combinations, respectively, to obtain N ratios of pixel density, wherein the ratio of pixel density is a ratio of pixel occupancy of one segment of the outer boundary image to one segment of the inner boundary image in each of the image combinations, and the pixel occupancy is a ratio of pixel points to total pixel points; and
the calculating unit is further used for calculating a quality of the grayscale image corresponding to the tissue mask image based on the ratio of pixel density.

Further, in a possible implementation of the present embodiment, as shown in FIG. 48, said first segmentation unit 23 is further used for:
inputting the registered image into an image segmentation model to obtain a segmentation result of individual pixel points in the registered image, wherein the segmentation result includes a pixel point belonging to a cell, a pixel point belonging to cell boundary, or a pixel point belonging to a image background; and
determining the pixel point belonging to a cell and the pixel point belonging to cell boundary in segmentation results as the pixel point belonging to a cell, and outputting a segmentation result of the registered image to obtain the cell mask image.

Further, in a possible implementation of the present embodiment, as shown in FIG. 48, said first segmentation unit 23 is further used for:
preprocessing the visualization image of gene expression to obtain a preprocessed image;
performing a binaryzation processing for the preprocessed image to obtain an initial mask image; and
based on the initial mask image, segmenting a connected component with a presence of cell adhesion using a distance transform-based watershed algorithm to obtain a segmented cell mask image.

Further, in a possible implementation of the present embodiment, as shown in FIG. 48, said first segmentation unit 23 is further used for:
determining a labeled region and a cell area corresponding to each tissue type of said target object based on the stained image;
determining an area of a sub-region to be processed in each labeled region based on the cell area, wherein the area of the sub-region to be processed is in a positive correlation with the cell area, and the sub-region to be processed characterizes the minimum processing unit of the corresponding labeled region;
performing a grid processing for the corresponding labeled region based on the area of each said sub-region to be processed to obtain gridded coordinates corresponding to each said labeled region; and
extracting data corresponding to the gridded coordinates from the visualization image of gene expression and superimposing the data to the gridded coordinates to obtain a cell processing result of the target object.

Further, in a possible implementation of the present embodiment, as shown in FIG. 48, said correction unit 24 is further used for:
classifying, based on the cell mask image, individual gene expression features of the visualization image of gene expression as a cellular gene expression feature located inside a cell and a background gene expression feature located outside the cell;
fitting a probability distribution model with individual gene expression features using a spatial location and a signal value of the gene expression feature;
obtaining, based on the probability distribution model, a probability value showing that a target background gene expression feature, which is located within a predetermined range of a target cell belongs to the target cell; and
outputting a correction result based on the probability value for correction of the background gene expression feature.

Further, in a possible implementation of the present embodiment, as shown in FIG. 48, said correction unit 24 is further used for:
determining an initial gene molecule belonging to a target cell and the background gene molecule unknown of the cell-belonging based on the visualization image of gene expression;
acquiring a coordinate information of the initial gene molecule;
determining center point coordinates of said target cell based on the coordinate information;
constructing a Voronoi diagram based on the center point coordinates and the coordinate information; and
determining the cell-belonging for the background gene molecule unknown of the cell-belonging within the Voronoi diagram.

Further, in a possible implementation of the present embodiment, as shown in FIG. 48, said correction unit 24 is further used for:
determining an initial molecule number of gene molecules belonging to a target cell and an initial boundary area of a target region occupied by the gene molecules belonging to the target cell in the visualization image of gene expression;
determining a density of the gene molecules belonging to the target cell based on the molecule number and the initial boundary area;
determining a molecule number of background gene molecules within a predetermined range of the target region and an area of the predetermined range;
determining a density of the background gene molecules based on the molecule number of the background gene molecules and the area of the predetermined range of the background gene molecule; and
determining the cell-belonging for the background gene molecule based on the density of the gene molecules versus the density of the background gene molecules.

Further, in a possible implementation of the present embodiment, as shown in FIG. 48, said correction unit 24 is further used for:
determining a target region occupied by al gene molecule belonging to a target cell in the visualization image of gene expression;
determining spatial coordinates of the background gene molecule unknown of the cell-belonging within a predetermined correction range of the target region and a signal value of a gene expression feature thereof;
inputting the spatial coordinates and the signal value of the gene expression feature into a probability distribution model, to obtain a probability value showing that the background gene molecule belongs to the target cell, wherein the probability distribution model is fitted with the spatial coordinates and the signal value of the gene expression feature for the gene molecule belonging to the target cell; and
determining the cell-belonging for the background gene molecule based on the probability score.

Further, in a possible implementation of the present embodiment, as shown in FIG. 48, said labeling unit 25 is used for:
staining the cell with the determined cell-belonging by a ssDNA staining solution and a cell boundary fluorescent staining solution, wherein the ssDNA staining solution includes a buffer and a ssDNA reagent, and the cell boundary fluorescent staining solution includes a fluorescent dye.

Further, in a possible implementation of the present embodiment, as shown in FIG. 48, said device further includes said detection unit 29 for:
obtaining a stained image to be tested or a visualization image of gene expression to be tested; and
inputting the stained image to be tested or the visualization image of gene expression to be tested into a directed object detection network to obtain an object detection result,
wherein the directed object detection network includes:
a backbone layer configured to perform a feature extraction on the stained image and the visualization image of gene expression, to obtain a plurality of feature images at different scales;
a neck layer configured to receive the plurality of feature images from the backbone layer and perform a cross-scale fusion for the plurality of feature images, to obtain a plurality of fused feature images; and
a head layer configured to perform a prediction for the plurality of fused feature images, to obtain the object detection result with respect to the stained image to be tested or the visualization image of gene expression to be tested,
wherein the object detection result includes: a prediction information of an object box coordinate, an object prediction information and an angle prediction information.

Optionally, a determination unit 210 is also included, wherein the determination unit 210 is used for:
wherein a plane where the stained image is located includes a first direction and a second direction perpendicular to the first direction, the stained image includes a plurality of first straight track lines transverse to the second direction and a plurality of second straight track lines transverse to the first direction

A step of sampling, includes:
performing first-direction equal-interval sampling for the stained image in a first direction by applying a first sampling strip to determine a first candidate point set, wherein each time of said first-direction equal-interval sampling includes:
calculating a cumulative sum of pixel values in a first direction for pixels within a sampled region of the stained image selected during this time of said first-direction equal-interval sampling by the first sampling strip, to obtain a series of first pixel value sums; and
determining a first extreme point of the series of first pixel value sums, and determining, for each first extreme point determined, a first candidate point obtained from this time of said first-direction equal-interval sampling, wherein a coordinate of the first candidate point in the first direction is determined according to a location of the first sampling strip in the first direction during this time of said first-direction equal-interval sampling, a coordinate of the first candidate point in the second direction is determined according to a coordinate in the second direction corresponding to a location of the first extreme point in the second direction,
wherein the first candidate point set consists of the first candidate points obtained from individual times of said first-direction equal interval sampling;
   performing second-direction equal interval sampling for the stained image in a second direction by applying a second sampling strip to determine a second candidate point set, wherein each time of said second-direction equal interval sampling includes:
calculating a cumulative sum of pixel values in a second direction for pixels within a sampled region of the stained image selected during this time of said second-direction equal-interval sampling by the second sampling strip, to obtain a series of second pixel value sums; and
determining a second extreme point of the series of second pixel value sums, and determining, for each second extreme point determined, a second candidate point obtained from this time of said second-direction equal interval sampling, wherein a coordinate of the second candidate point in the second direction is determined according to a location of the second sampling strip in the second direction during this time of said second-direction equal-interval sampling, a coordinate of the second candidate point in the first direction is determined according to a coordinate in the first direction corresponding to a location of the second extreme point in the first direction,
wherein the second candidate point set consists of the second candidate points obtained from individual times of said second-direction equal interval sampling.

A step of grouping, includes:
dividing the first candidate points in the first candidate point set into a plurality of first candidate point groups; and
dividing the second candidate points in the second candidate point set into a plurality of second candidate point groups.

A step of linear fitting, includes:
performing a first linear fit for the first candidate points in individual first candidate point groups to obtain respective first linear analytic formulas; and
performing a second linear fit for the second candidate points in individual second candidate point groups to obtain respective second linear analytic formulas.

A step of coordinate calculation, includes:
calculating coordinates of respective intersections of individual first linear formulas with individual second linear formula.

Further, in a possible implementation of the present embodiment, as shown in FIG. 48, said device further includes an analyzing unit 211 for:
obtaining a first image of the biological sample, wherein the first image includes a stained cell boundary of the biological sample;
obtaining a second image of the biological sample, wherein the second image includes a stained genetic material of the biological sample;
obtaining information about a gene expression level of the biological sample;
registering the second image with the first image to obtain a registered image of the second image with the first image;
registering the second image with the information about the gene expression level to obtain a registered image of the second image with the information about the gene expression level;
obtaining a registered image of the first image with the information about the gene expression level based on the registered image of the second image with the first image and the registered image of the second image with the information about the gene expression level;
obtaining a cell boundary of the biological sample based on the registered image of the first image with the information about the gene expression level;
obtaining gene expression information of a cell of the biological sample based on the cell boundary and the registered image of the first image with the information about the gene expression level.

According to embodiments of the present disclosure, the present disclosure also provides an electronic device, a readable storage medium, and a computer program product.

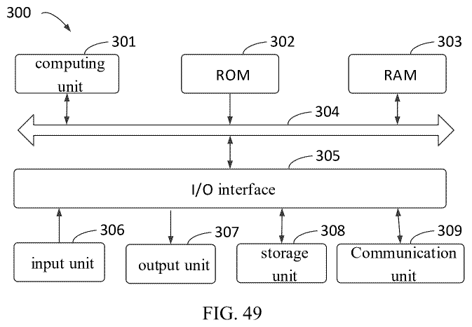

FIG. 49 illustrates a schematic frame diagram of an exemplary electronic device 300, which may implement embodiments of the present disclosure. The electronic device is intended to represent various forms of digital computers, such as, a laptop, desktop computer, workstation, personal digital assistants, servers, blade servers, mainframe computers, and other suitable computers. Electronic devices may also denote various forms of mobile devices such as, personal digital processer, cellular phones, smart phones, wearable devices, and other similar computer devices. The components, their connections and relationships, and their functions shown herein are intended as examples only, and not to limit the implementations of the present disclosure described and/or claimed herein.

As shown in FIG. 49, the device 300 includes a computing unit 301 which may perform various appropriate operations and processing based on a computer program stored in a ROM (Read-Only Memory) 302 or a computer program loaded from a storage unit 308 into a RAM (Random Access Memory) 303. In the RAM 303, various programs and data required for operation of the device 300 may also be stored. The computing unit 301, the ROM 302, and the RAM 303 are connected to each other via the bus 304, and the I/O (Input/Output) interface 305 is also connected to the bus 304.

A plurality of components in the device 300 connected to the I/O interface 305, includes: an input unit 306, such as a keyboard, mouse, etc.; an output unit 307, such as various types of displays, speakers, etc.; a storage unit 308, such as a disk, CD-ROM, etc.; and a communication unit 309, such as a network card, modem, wireless communication transceiver, etc. The communication unit 309 allows the device 300 to exchange information/data with other devices via a computer network such as the Internet and/or various telecommunication networks.

The computing unit 301 may be various general-purpose and/or special-purpose processing components with processing and computational capabilities. Some examples of computing units 301 include, but are not limited to, CPUs (Central Processing Units), GPU (Graphic Processing Units), various specialized AI (Artificial Intelligence) computing chips, various AI (Artificial Intelligence) computing chips, various computing units for running machine learning model algorithms, DSP (Digital Signal Processors), and any appropriate processors, controllers, microcontrollers, and the like. Computing unit 301 performs the various methods and processing described above, such as the method for obtaining spatial omics single cell data. For example, in some embodiments, the method for obtaining spatial omics single cell data may be implemented as a computer program that is tangibly contained in a machine-readable medium, such as a storage unit 308. In some embodiments, part or all of the computer program may be loaded and/or installed on the device 300 via the ROM 302 and/or the communication unit 309. When the computer program is loaded into RAM 303 and executed by computing unit 301, one or more steps of the method described above may be performed. Alternatively, in other embodiments, the computing unit 301 may be configured to perform the method for obtaining spatial omics single cell data described above by any other suitable means (e.g., with the aid of firmware).

Various implementations of the systems and techniques described above herein can be found in digital electronic circuit and systems, integrated circuit systems, FPGA (Field Programmable Gate Array), ASIC (Application-Specific Integrated Circuit), ASSP (Application Specific Standard Product), SOC (System On Chip), CPLD (Complex Programmable Logic Device), computer hardware, firmware, software, and/or combinations thereof. These various embodiments may include: implementation in one or more computer programs that may be executed and/or interpreted on a programmable system including at least one programmable processor. The programmable processor may be a special-purpose or general-purpose programmable processor, that may receive data and instructions from the storage system, at least one input device, and the at least one output device, and that may transmit the data and instructions to the storage system, the at least one input device, and the at least one output device.

The program code for implementing the methods of the present disclosure may be written in any combination of one or more programming languages. Such program code may be provided to a processor or controller of a general-purpose computer, a special-purpose computer, or other programmable data-processing device such that enabling the functions/operations set forth in the flowchart and/or the block diagram to be performed when the program code is executed by the processor or controller. The program code may be executed entirely on the machine, partially on the machine, partially on the machine as a stand-alone software package and partially on a remote machine or entirely on a remote machine or server.

In the context of the present disclosure, a machine-readable medium may be any tangible medium including or storing programs for use in an instruction executed system, device, or apparatus or by for use in conjunction with an instruction executed system, device, or apparatus. The machine-readable medium may be a machine-readable signal medium or a machine-readable storage medium. The machine-readable medium may include, but is not limited to, of an electronic, magnetic, optical, electromagnetic, infrared, or semiconductor system, device, or apparatus, or any suitable combination of the foregoing. More specific examples of machine-readable storage media may include electrical connections based on one or more wires, portable computer disks, hard disks, RAM, ROM, EPROM (Electrically Programmable Read-Only-Memory), or flash memory, optical fiber, CD-ROM (Compact Disc Read-Only Memory), optical storage device, magnetic storage device, or any suitable combination thereof.

To provide interaction with a user and the systems and implementation of techniques described herein on a computer, the computer includes: a display device, such as a CRT (Cathode-Ray Tube) or LCD (Liquid Crystal Display) monitor, for displaying information to the user; and a keyboard and pointing device (e.g., a mouse or trackball), through which the user can provide input to the computer. Other types of devices may also be used to provide interaction with the user; for example, the feedback provided to the user may be any form of sensory feedback (e.g., visual feedback, auditory feedback, or haptic feedback); and the input from the user may be received in any form, including acoustic, voice, or haptic input.

The systems and techniques described herein may be implemented in a computing system that includes a back-end component (e.g., as a data server), or a computing system that includes a middleware component (e.g., an application server), or a computing system that includes a front-end component (e.g., a user's computer that has a graphical user interface or a web browser through which a user can interact with the systems and techniques described herein), or in a computing system that includes any combination of the back-end, middleware, or front-end components. The components of the system may be interconnected via digital data communications (e.g., a communications network) in any form or medium. Examples of communication networks include LANs (Local Area Networks), WANs (Wide Area Networks), the Internet, and blockchain networks.

A computer system may include a client and a server. Clients and servers are generally remote from each other and typically interact over a communication network. The client-server relationship is generated by a computer program that runs on a corresponding computer and has a client-server relationship with each other. A server may be a cloud server, also known as a cloud computing server or cloud host, which is a host product in the cloud computing service system to solve the shortcomings of traditional physical hosts and VPS (Virtual Private Server) services, i.e. difficulty in management and weak business extensibility. Servers can also be servers for distributed systems, or servers that incorporate blockchain.

Among them, it should be noted that artificial intelligence is a discipline that studies how to simulate certain human thinking processes and intelligent behaviors (such as learning, reasoning, thinking, planning, etc.) with computers, encompassing both hardware and software technologies. Artificial intelligence hardware technologies generally include sensors, specialized AI chips, cloud computing, distributed storage, and big data processing technologies; artificial intelligence software technologies mainly include computer vision technology, speech recognition technology, natural language processing technology, as well as machine learning/deep learning, big data processing technologies, and knowledge graph technologies.

It will be appreciated that various forms of the process shown above may be used by reordering, adding or deleting any step. For example, the steps recited in the present disclosure may be performed in parallel or sequentially or in a different order as long as they arrive at the results desired by the technical solutions disclosed in the present disclosure, and are not limited herein.

The scope of protection of the present disclosure is not limited to the specific embodiments that has been described above. It would be appreciated by those skilled in the art that changes, various modifications, combinations, sub-combinations and alternatives may be made according to the design requirements and other factors. Any changes, equivalent alternatives, and modifications made within the spirit and principles of the present disclosure shall be should be included in the embodiments in the scope of the present disclosure.

1. A method for obtaining spatial omics single cell data, the method comprising:
obtaining a stained image of a biological sample and a visualization image of gene expression of the biological sample;
performing image registration for the stained image and the visualization image of gene expression to obtain a registered image;
performing cell segmentation for the registered image to obtain a cell mask image; and
determining cell-belonging for a background gene molecule unknown of the cell-belonging in the visualization image of gene expression based on the cell mask image, and labeling a cell with the determined cell-belonging for the background gene molecule, to obtain an expression matrix of spatial omics molecules with the cell label.

2. The method of claim 1, wherein obtaining the stained image of the biological sample comprises:
obtaining the stained image of the biological sample with a microscopy; or
obtaining a stitched stained image of the biological sample with a microscope, wherein the stitched stained image is stitched from a plurality of sub-stained images; or
obtaining a plurality of sub-stained images of the biological sample with a microscopy, and stitching the plurality of sub-stained images to obtain the stitched stained image.

3. The method of claim 2, wherein obtaining the stained image of the biological sample comprises:
obtaining the plurality of sub-stained images of the biological sample with a microscopy;
performing first stitching for the plurality of sub-stained images of the biological sample to obtain a first stained image;
determining whether to perform a second stitching for the plurality of sub-stained images of the biological sample based on an image quality of the first stained image;
identifying the first stained image as the stained image in response to a negative result of the determination; and
performing the second stitching for the plurality of sub-stained images of the biological sample to obtain the stained image in response to a positive result of the determination.

4. The method of claim 3, wherein obtaining the plurality of sub-stained images of the biological sample with the microscopy, and stitching the plurality of sub-stained images to obtain the stitched stained image comprise:
determining an overlapping region between each of the plurality of sub-stained images and a template image;
obtaining a plurality of overlapping sub-image pairs by matching a plurality of overlapping sub-stained images and a plurality of overlapping sub-template images, wherein a section corresponding to the determined overlapping region in each of the plurality of sub-stained images and a section corresponding to the determined overlapping region in the template image are cropped respectively, to provide the plurality of overlapping sub-stained images and the plurality of overlapping sub-template images that are identical by numbers;
obtaining an offset between each of the plurality of sub-stained images and the corresponding sub-template image by performing frequency domain calculations on the plurality of overlapping sub-image pairs; and
translating each of the plurality of sub-stained images by the offset toward the corresponding sub-template image, and stitching the plurality of sub-stained images together with the template image.

5. The method of claim 3, wherein obtaining the plurality of sub-stained images of the biological sample with the microscopy, and stitching the plurality of sub-stained images to obtain the stitched stained image comprise:
obtaining the plurality of sub-stained images and template information about a track line or a track cross for the biological sample;
performing a pre-stitching for the plurality of sub-stained images to obtain respective pre-stitched coordinates of the plurality of sub-stained images;
selecting at least one first reference image from the plurality of sub-stained images according to a feature of the track line or the track cross;
deriving a global template according to the template information of the biological sample and the pre-stitched coordinates of the first reference image;
for a second reference image in the plurality of sub-stained images, calculating an offset between pre-stitched coordinates of the second reference image and corresponding template coordinates of the second reference image in the global template, wherein the second reference image is different from the first reference image; and
adjusting the coordinates of the second reference image by the offset and stitching the second reference image for generation of the stitched stained image.

6. The method of claim 1, wherein performing image registration for the stained image and the visualization image of gene expression to obtain the registered image comprises:
obtaining a track line in the stained image and rotating the stained image in an angle between the track line and a horizontal direction, wherein the stained image and the visualization image of gene expression each comprise a plurality of track lines and/or a plurality of track crosses, two of the track lines intersect to form the track crosses, and each of the plurality of track lines is corresponded with an index number;
obtaining a third reference image by scaling the stained image, wherein the third reference image has the same scale as the visualization image of gene expression;
determining an object offset based on the visualization image of gene expression and the third reference image; and
translating the third reference image by the object offset to obtain the registered image.

7. The method of claim 6, wherein after obtaining the third reference image by scaling the stained image, the method further comprises:
determining a similarity between the third reference image and the visualization image of gene expression; and
taking a position with a maximum similarity between the third reference image and the visualization image of gene expression as a result of a coarse registration.

8. The method of claim 1, wherein performing image registration for the stained image and the visualization image of gene expression to obtain the registered image comprises:
performing a preprocessing for the stained image to obtain a candidate image set;
determining a binary image set corresponding to the candidate image set;
determining a track cross pair(s) having a correspondence between the binary image set and the visualization image of gene expression, based on a similarity between the binary image set and the visualization image of gene expression;
determining an offset information based on the track cross pair; and
processing an object image based on the offset information to obtain the registered image, wherein the object image is an image from the candidate image set and corresponding to the track cross pair.

9. (canceled)

10. The method of claim 1, wherein before performing the cell segmentation for the registered image to obtain the cell mask image, the method further comprises:
performing a tissue segmentation for the registered image to obtain a tissue mask image,
wherein performing the tissue segmentation for the registered image to obtain the tissue mask image comprises:
obtaining a gene expression of an object tissue to be segmented in the registered image to determine a gene expression matrix of the registered image, wherein the gene expression in the gene expression matrix is arranged according to a pixel position;
determining image data by assigning the gene expression at each pixel position in the gene expression matrix to a corresponding pixel position in the registered image;
performing an image processing for the image data to determine a foreground pixel point of the image data; and
labeling connected components for the image data based on the foreground pixel point to obtain the tissue mask image.

11. The method of claim 10, wherein after performing the tissue segmentation for the registered image to obtain the tissue mask image, the method further comprises:
obtaining an outer boundary image and an inner boundary image in a grayscale image corresponding to the tissue mask image by calculating respectively;
dividing the outer boundary image and the inner boundary image into N of segments respectively, and matching each segment of the outer boundary image with each segment of the inner boundary image by pair to obtain N image combinations, wherein each of the image combinations comprises one segment of the outer boundary image and one segment of the inner boundary image;
calculating a ratio of pixel density between one segment of the outer boundary image and one segment of the inner boundary image in each of the image combinations, respectively, to obtain N ratios of pixel density, wherein the ratio of pixel density is a ratio of pixel occupancy of one segment of the outer boundary image to one segment of the inner boundary image in each of the image combinations, and the pixel occupancy is a ratio of pixel points to total pixel points; and
calculating a quality of the grayscale image corresponding to the tissue mask image based on the ratio of pixel density.

12. The method of claim 1, wherein performing the cell segmentation for the registered image to obtain the cell mask image comprises:
inputting the registered image into an image segmentation model to obtain a segmentation result of individual pixel points in the registered image, wherein the segmentation result comprises a pixel point belonging to a cell, a pixel point belonging to cell boundary, or a pixel point belonging to an image background; and
determining the pixel point belonging to a cell and the pixel point belonging to cell boundary in segmentation results as the pixel point belonging to a cell, and outputting a segmentation result of the registered image to obtain the cell mask image.

13. The method of claim 1, wherein performing the cell segmentation for the registered image to obtain the cell mask image comprises:
preprocessing the visualization image of gene expression to obtain a preprocessed image;
performing a binaryzation processing for the preprocessed image to obtain an initial mask image; and
based on the initial mask image, segmenting a connected component with a presence of cell adhesion using a distance transform-based watershed algorithm to obtain a segmented cell mask image.

14. The method of claim 1, wherein performing the cell segmentation for the registered image to obtain the cell mask image comprises:
determining a labeled region and a cell area corresponding to each tissue type of a target object based on the stained image;
determining an area of a sub-region to be processed in each labeled region based on the cell area, wherein the area of the sub-region to be processed is in a positive correlation with the cell area, and the sub-region to be processed characterizes the minimum processing unit of the corresponding labeled region;
performing a grid processing for the corresponding labeled region based on the area of each said sub-region to be processed to obtain gridded coordinates corresponding to each said labeled region; and
extracting data corresponding to the gridded coordinates from the visualization image of gene expression and superimposing the data to the gridded coordinates to obtain a cell processing result of the target object.

15. The method of claim 1, wherein determining the cell-belonging for the background gene molecule unknown of the cell-belonging in the visualization image of gene expression based on the cell mask image comprises:
classifying, based on the cell mask image, individual gene expression features of the visualization image of gene expression as a cellular gene expression feature located inside a cell and a background gene expression feature located outside the cell;
fitting a probability distribution model with individual gene expression features using a spatial location and a signal value of the gene expression feature;
obtaining, based on the probability distribution model, a probability value showing that a target background gene expression feature, which is located within a predetermined range of a target cell belongs to the target cell; and
outputting a correction result based on the probability value for correction of the background gene expression feature.

16. The method of claim 1, wherein determining the cell-belonging for the background gene molecule unknown of the cell-belonging in the visualization image of gene expression based on the cell mask image comprises:
determining an initial gene molecule belonging to a target cell and the background gene molecule unknown of the cell-belonging based on the visualization image of gene expression;
acquiring a coordinate information of the initial gene molecule;
determining center point coordinates of said target cell based on the coordinate information;
constructing a Voronoi diagram based on the center point coordinates and the coordinate information; and
determining the cell-belonging for the background gene molecule unknown of the cell-belonging within the Voronoi diagram.

17. The method of claim 1, wherein determining the cell-belonging for the background gene molecule unknown of the cell-belonging in the visualization image of gene expression based on the cell mask image comprises:
determining an initial molecule number of gene molecules belonging to a target cell and an initial boundary area of a target region occupied by the gene molecules belonging to the target cell in the visualization image of gene expression;
determining a density of the gene molecules belonging to the target cell based on the initial molecule number and the initial boundary area;
determining a molecule number of background gene molecules within a predetermined range of the target region and an area of the predetermined range;
determining a density of the background gene molecules based on the molecule number of the background gene molecules and the area of the predetermined range of the background gene molecule; and
determining the cell-belonging for the background gene molecule based on the density of the gene molecules versus the density of the background gene molecules.

18. The method of claim 1, wherein determining the cell-belonging for the background gene molecule unknown of the cell-belonging in the visualization image of gene expression based on the cell mask image comprises:
determining a target region occupied by a gene molecule belonging to a target cell in the visualization image of gene expression;
determining spatial coordinates of the background gene molecule unknown of the cell-belonging within a predetermined correction range of the target region and a signal value of a gene expression feature thereof;
inputting the spatial coordinates and the signal value of the gene expression feature into a probability distribution model, to obtain a probability value showing that the background gene molecule belongs to the target cell, wherein the probability distribution model is fitted with the spatial coordinates and the signal value of the gene expression feature for the gene molecule belonging to the target cell; and
determining the cell-belonging for the background gene molecule based on the probability score.

19. (canceled)

20. The method of claim 5, further comprising:
obtaining a stained image to be tested or a visualization image of gene expression to be tested; and
inputting the stained image to be tested or the visualization image of gene expression to be tested into a directed object detection network to obtain an object detection result,
wherein the directed object detection network comprises:
a backbone layer configured to perform a feature extraction on the stained image and the visualization image of gene expression, to obtain a plurality of feature images at different scales;
a neck layer configured to receive the plurality of feature images from the backbone layer and perform a cross-scale fusion for the plurality of feature images, to obtain a plurality of fused feature images; and
a head layer configured to perform a prediction for the plurality of fused feature images, to obtain the object detection result with respect to the stained image to be tested or the visualization image of gene expression to be tested,
wherein the object detection result comprises: a prediction information of an object box coordinate, an object prediction information and an angle prediction information.

21. The method of claim 5, wherein a plane where the stained image is located comprises a first direction and a second direction perpendicular to the first direction, the stained image comprises a plurality of first straight track lines transverse to the second direction and a plurality of second straight track lines transverse to the first direction,
wherein coordinates of points in the stained image are determined by:
a step of sampling, comprising:
performing first-direction equal-interval sampling for the stained image in a first direction by applying a first sampling strip to determine a first candidate point set, wherein each time of said first-direction equal-interval sampling comprises:
calculating a cumulative sum of pixel values in a first direction for pixels within a sampled region of the stained image selected during this time of said first-direction equal-interval sampling by the first sampling strip, to obtain a series of first pixel value sums; and
determining a first extreme point of the series of first pixel value sums, and determining, for each first extreme point determined, a first candidate point obtained from this time of said first-direction equal-interval sampling, wherein a coordinate of the first candidate point in the first direction is determined according to a location of the first sampling strip in the first direction during this time of said first-direction equal-interval sampling, a coordinate of the first candidate point in the second direction is determined according to a coordinate in the second direction corresponding to a location of the first extreme point in the second direction,
wherein the first candidate point set consists of the first candidate points obtained from individual times of said first-direction equal interval sampling;
performing second-direction equal interval sampling for the stained image in a second direction by applying a second sampling strip to determine a second candidate point set, wherein each time of said second-direction equal interval sampling comprises:
calculating a cumulative sum of pixel values in a second direction for pixels within a sampled region of the stained image selected during this time of said second-direction equal-interval sampling by the second sampling strip, to obtain a series of second pixel value sums; and
determining a second extreme point of the series of second pixel value sums, and determining, for each second extreme point determined, a second candidate point obtained from this time of said second-direction equal interval sampling, wherein a coordinate of the second candidate point in the second direction is determined according to a location of the second sampling strip in the second direction during this time of said second-direction equal-interval sampling, a coordinate of the second candidate point in the first direction is determined according to a coordinate in the first direction corresponding to a location of the second extreme point in the first direction,
wherein the second candidate point set consists of the second candidate points obtained from individual times of said second-direction equal interval sampling;
a step of grouping, comprising:
dividing the first candidate points in the first candidate point set into a plurality of first candidate point groups; and
dividing the second candidate points in the second candidate point set into a plurality of second candidate point groups;
a step of linear fitting, comprising:
performing a first linear fit for the first candidate points in individual first candidate point groups to obtain respective first linear analytic formulas; and
performing a second linear fit for the second candidate points in individual second candidate point groups to obtain respective second linear analytic formulas; and
a step of coordinate calculation, comprising:
calculating coordinates of respective intersections of individual first linear formulas with individual second linear formula.

22. The method of claim 1, wherein after labeling a cell with the determined cell-belonging for the background gene molecule to obtain an expression matrix of spatial omics molecules with the cell label, the method further comprises:
obtaining a first image of the biological sample, wherein the first image comprises a stained cell boundary of the biological sample;
obtaining a second image of the biological sample, wherein the second image comprises a stained genetic material of the biological sample;
obtaining information about a gene expression level of the biological sample;
registering the second image with the first image to obtain a registered image of the second image with the first image;
registering the second image with the information about the gene expression level to obtain a registered image of the second image with the information about the gene expression level;
obtaining a registered image of the first image with the information about the gene expression level based on the registered image of the second image with the first image and the registered image of the second image with the information about the gene expression level;
obtaining a cell boundary of the biological sample based on the registered image of the first image with the information about the gene expression level; and
obtaining gene expression information of a cell of the biological sample based on the cell boundary and the registered image of the first image with the information about the gene expression level.

23. (canceled)

24. An electronic device, comprising:
a processor, and
a memory communicatively connected to the processor,
wherein the memory having stored therein instructions that, when executed by the processor of the electronic device, causes the electronic device to perform the method according to claim 1.

25.-26. (canceled)
